(19) United States
(12) Patent Application Publication
(10) Pub. No.: US 2015/0159184 A1
(43) Pub. Date: Jun. 11, 2015

(54) Genetically Engineered Microorganisms for the Production of Poly-4-Hydroxybutyrate

(71) Applicant: Metabolix, Inc., Cambridge, MA (US)

(72) Inventors: Thomas M. Ramseier, Newton, MA (US); Christopher W.J. McChalicher, Arlington, MA (US); William R. Farmer, Concord, MA (US); Zhigang Zhang, Watertown, MA (US); Dong-Eun Chang, Newton, MA (US); Jeff Bickmeier, Arlington, MA (US); Julie Beaulieu, Saugus, MA (US); Catherine Morse, Melrose, MA (US)

(21) Appl. No.: 14/386,728

(22) PCT Filed: Mar. 4, 2013
(86) PCT No.: PCT/US2013/028913

§ 371 Date: Sep. 19, 2014

Related U.S. Application Data
(60) Provisional application No. 61/613,388, filed on Mar. 20, 2012.

(51) Int. Cl. C12P 7/62; C12N 9/12; C12N 9/04; C12N 9/00; C12N 9/88; C12N 9/02

(52) U.S. Cl. CPC C12P 7/625; C12N 9/88; C12N 9/0008; C12N 9/0006; C12N 9/93; C12N 9/001; C12N 9/1205; C12Y 401/01071; C12Y 102/01075; C12Y 101/01002; C12Y 602/01004; C12Y 604/01001; C12Y 103/01006; C12Y 207/01086

(57) ABSTRACT

Methods and genetically engineered hosts for the production of poly-4-hydroxybutrate and 4-carbon products are described herein.

### RELATED APPLICATIONS

This application claims the benefit of U.S. Provisional Application No. 61/613,388, filed on Mar. 20, 2012. The entire teachings of the above application is incorporated herein by reference.

This application incorporates by reference the Sequence Listing contained in the following ASCII text file being submitted concurrently herewith:
a. File name: 46141007001 SEQ.txt; created Feb. 26, 2013, 76.3975 KB in size.

### BACKGROUND OF THE INVENTION

Biobased, biodegradable polymers such as polyhydroxyalkanoates (PHAs), have been produced in such diverse biomass systems as plant biomass, microbial biomass (e.g., bacteria including cyanobacteria, yeast, fungi) or algae biomass. Genetically-modified biomass systems have recently been developed which produce a wide variety of biodegradable PHA polymers and copolymers (Lee (1996), Biotechnology & Bioengineering 49:1-14; Braunegg et al. (1998), J. Biotechnology 65:127-161; Madison, L. L. and Huisman, G. W. (1999), Metabolic Engineering of Poly-3-Hydroxyalkanoates; From DNA to Plastic, in: Microbiol. Mol. Biol. Rev. 63:21-53).

There has also recently been progress in the development of biomass systems that produce “green” chemicals such as gamma-butyrolactone, (Metabolix), 1,3-propanediol (Dupont's BioPDO®), 1,4-butanediol (Genomatica) and succinic acid (Bioamber) to name a few. Analogous to the biobased PHA polymers, these biobased chemicals have been produced by genetically-modified biomass systems which utilize renewable feedstocks, have lower carbon footprints and reportedly lower production costs as compared to the traditional petroleum chemical production methods.

With dwindling petroleum resources, increasing energy prices, and environmental concerns, development of energy efficient biorefinery processes to produce biobased chemicals from renewable, low cost, carbon resources offers a unique solution to overcoming the increasing limitations of petroleum-based chemicals.

However, a disadvantage of these methods is the low amount of polymer in the biomass that further results in low amounts of the subsequent desired products. Thus, a need exists to produce genetically modified organisms with increased amounts of polymer (e.g., poly-4-hydroxybuytyrate) that in turn can be further processed to green chemicals that overcome the disadvantages of low yield, cell toxicity, and low purity of the current methodologies.

### SUMMARY OF THE INVENTION

The invention generally relates to methods of increasing the production of a 4-carbon (C4) product or a polymer of 4-carbon monomers from a renewable feedstock, comprising providing a genetically modified organism having a reduced activity of alpha-ketoglutarate dehydrogenase such that growth is impaired as compared to a wild-type organism without the reduced activity; and providing one or more genes that are stably expressed that encodes an enzyme with an activity catalyzing the decarboxylation of alpha-ketoglutarate to succinic semialdehyde; wherein growth is improved and the carbon flux from the renewable feedstock 4-carbon (C4) product or a polymer of 4-carbon monomers is increased.

In certain embodiments of any of the aspects of the invention, the pathway is a poly-4-hydroxybutyrate (P4HB) pathway or a 1,4 butanediol (BDO) pathway.

The invention also pertains to increasing the amount of poly-4-hydroxybutyrate in a genetically engineered organism by stably incorporating one or more genes that express enzymes for increased production of the poly-4-hydroxybutyrate. An exemplary pathway for production of P4HB is provided in FIG. 1 and it is understood that additional enzymatic changes that contribute to this pathway can also be introduced or suppressed for a desired production of P4HB.

In a first aspect, a method of increasing the production of a 4-carbon (C4) product or a polymer of 4-carbon monomers from a renewable feedstock, comprising

a) providing a genetically modified organism having a modified metabolic C4 pathway, and
b) providing one or more genes that are stably expressed that encodes one or more enzymes having an activity of i) catalyzing the decarboxylation of alpha-ketoglutarate to succinic semialdehyde; ii) catalyzing the conversion of malonyl CoA to malonate semialdehyde iii) catalyzing the conversion of L-lactaldehyde to L-1,2-propanediol and having increased resistance to oxidative stress; iv) catalyzing fumarate to succinate; v) catalyzing the carboxylation of pyruvate; or vi) catalyzing NADH to NADPH; wherein the production of the product or polymer is improved compared to a wild type or the modified organism of step a) and/or the carbon flux from the renewable feedstock 4-carbon (C4) product or a polymer of 4-carbon monomers is increased is described.

In a first embodiment of the first aspect, the invention pertains to a method of producing an increase of poly-4-hydroxybutyrate in a genetically modified organism (recombinant host) having a poly-4-hydroxybutyrate pathway. The enzymes of the first aspect catalyze one of the reactions in the poly-4-hydroxybutyrate pathway, for example, the enzyme malonyl-CoA reductase is also capable of converting to Suc-CoA to succinic semialdehyde (SSA) (Reaction 5, of FIG. 1) and does not promote the conversion to 3-hydroxypropionate; the oxidative stress-resistant 1,2 propanediol oxidoreductase is also capable of converting SSA to 4-hydroxybutyrate (Reaction 8 of FIG. 1); the NADH-dependent fumarate reductase is also capable of converting fumarate to succinate, reaction 14 of FIG. 1; and a pyruvate carboxylase is capable of converting pyruvate to form oxaloacetate. Additionally, in the first aspect, incorporating one or more NADH kinases in the pathway increases intracellular NADPH concentrations and increases the level of poly 4-hydroxybutyrate (Reaction 17 of FIG. 1.).

In a second embodiment of the first aspect or first embodiment, one or more genes that are stably expressed encode one or more enzymes selected from: alpha-ketoglutarate decarboxylase, 2-oxoglutarate decarboxylase, malonyl-CoA reductase, NADH-dependent fumarate reductase, oxidative stress-resistant 1,2 propanediol oxidoreductase, pyruvate carboxylase and NADH kinase.

In a third embodiment of the first aspect, or first or second embodiment, the one or more enzyme is selected from an alpha-ketoglutarate decarboxylase from Pseudonocardia dioxanivorans or mutants and homologues thereof; an 2-oxoglutarate decarboxylase enzyme from Synechococcus sp. PCC 7002 or mutants and homologues thereof; a malonyl-CoA reductase from Metallosphaera sedula or mutants and homologues thereof; a malonyl-CoA reductase from Sulfolous tokodaii or mutants and homologues thereof; an oxidative stress-resistant 1,2 propanediol oxidoreductase from E. coli, mutants and homologues thereof; an NADH-dependent fumarate reductase from Trypanosoma brucei, mutants and homologues thereof; a pyruvate carboxylase from L. lactis, mutants and homologues thereof and an NADH kinase from Aspergillus nidulans, mutants and homologues thereof.

In a fourth embodiment of the first aspect, or first, second or third embodiment, the method includes stably incorporating in the organism's genome of a gene encoding an alpha-ketoglutarate decarboxylase or a 2-oxoglutarate decarboxylase enzyme. The alpha-ketoglutarate decarboxylase or 2-oxoglutaratedecarboxylase catalyzes the decarboxylation of alpha-ketoglutarate to succinic semialdehyde and increases the amount of poly-4-hydroxybutyrate in the organism by providing another enzyme reaction to succinic semialdehyde.

In a second aspect of the first aspect or the first, second, third or fourth embodiment, the host organism used to produce the biomass containing P4HB has been genetically modified by introduction of genes and/or deletion of genes in a wild-type or genetically engineered P4HB production organism creating strains that synthesize P4HB from inexpensive renewable feedstocks. In a third aspect of the invention or of the first or second aspect or any of the first, second, third or forth embodiments, the alpha-ketoglutarate decarboxylase is from Pseudonocardia dioxanivorans or mutants and homologues thereof or the 2-oxoglutaratedecarboxylase enzyme is from Synechococcus sp. PCC 7002 or mutants and homologues thereof.

In a fourth aspect of the invention or of the first, second or third aspect or any of the first, second, third or forth embodiments, the alpha-ketoglutarate decarboxylase from P. dioxanivorans comprises a mutation of an alanine to threonine at amino acid position 887.

In a fifth aspect of the invention or of the first, second, third or fourth aspect or any of the first, second, third or forth embodiments, the genetically engineered organism further has a stably incorporated gene encoding a succinate semialdehyde dehydrogenase that converts succinyl-CoA to succinic semialdehyde.

In a sixth aspect of the invention or of the first, second, third, fourth or fifth aspect or any of the first, second, third or forth embodiments, the succinate semialdehyde dehydrogenase is from Clostridium kluyveri or homologues thereof.

In a fifth embodiment of the invention or of the first, second, third, fourth, fifth, or six aspect or any of the first, second, third or forth embodiments, the genetically engineered organism having a poly-4-hydroxybutyrate pathway has an inhibiting mutation in its CoA-independent NAD-dependent succinic semialdehyde dehydrogenase gene or its CoA-independent NADP-dependent succinic semialdehyde dehydrogenase gene, or having inhibiting mutations in both genes, and having stably incorporated one or more genes encoding one or more enzymes selected a succinate semialdehyde dehydrogenase wherein the succinate semialdehyde dehydrogenase converts succinyl-CoA to succinic semialdehyde, a succinic semialdehyde reductase wherein the succinic semialdehyde reductase converts succinic semialdehyde to 4-hydroxybutyrate, and a polyhydroxyalkanoate synthase wherein the polyhydroxyalkanoate synthase polymerizes 4-hydroxybutyryl-CoA to poly-4-hydroxybutyrate.

In a sixth embodiment of the first, second, third, forth, fifth, sixth aspects or a further embodiment of the first embodiment, second embodiment, third embodiment, forth embodiment or fifth embodiment, the organism has a disruption and or reduction in the gene product in one or more gene selected from yneI, gabD, pykF, pykA, astD and SucCD.

The disruption or reduction in the gene product results in a decreased amount of product or the activity of the enzyme. For example, it was found that a reduction in the endogenous expression of SucCD, reduced the amount of product, succinyl-CoA synthetase and favorably allowed for the production of an increased amount of P4HB production. The reduction can be a decreased amount of product or activity. For example, a 3 percent to 25 percent reduction in activity, or a 25-95% reduction in activity, when compared to a gene and product having wild-type amounts of product or expression.

In a seventh embodiment, the method of the first, second, third, fourth, fifth, or sixth aspect or of the first, second embodiment, third, fourth, fifth or sixth embodiment, wherein the methods further includes an initial step of culturing a genetically engineered organism with a renewable feedstock to produce a 4-hydroxybutyrate biomass.

In a eighth embodiment, the method of the first, second, third, fourth, fifth, or sixth aspect or of the first, second embodiment, third, fourth, fifth, sixth or seventh embodiments, the methods include a source of the renewable feedstock that is selected from glucose, levoglucosan, fructose, sucrose, arabinose, maltose lactose xylose, fatty acids, vegetable oils, biomass derived synthesis gas, and methane originating from landfill gas, methanol derived from methane or a combination thereof. In a particular embodiment of any of the six aspects or of the eight embodiments, the feedstock is glucose or levoglucosan.

In an eighth embodiment, the method of the first, second, third, fourth, fifth, or sixth aspect or of the first, second embodiment, third, fourth, fifth, sixth or seventh embodiments, the organism is bacteria, yeast, fungi, algae, cyanobacteria, or a mixture of any two or more thereof.

The bacteria for use in the methods of the eight embodiment include but are not limited to E. coli, Ralstonia eutropha, Zoogloea ramigera, Allochromatium vinosum, Rhodococcus ruber, Delfia acidovorans, Aeromonas caviae, Synechocystis sp. PCC 6803, Synechococcus elongatus PCC 7942, Thiocapsa pfenigii, Bacillus megaterium, Acinetobacter baumannii, Acinetobacter baylyi, Clostridium kluyveri, Methylobacterium extorquens, Nocardia corralina, Nocardia salmonicolor, Pseudomonas fluorescens, Pseudomonas oleovorans, Pseudomonas sp. 6-19, Pseudomonas sp. 61-3 and Pseudomonas putida, Rhodobacter sphaeroides, Alcaligenes latus, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, and Clostridium acetobutylicum.

Exemplary yeasts or fungi for use in the methods including the eight embodiment include but are not limited to Saccharomyces cerevisiae, Schizosaccharomyces pombe, Kluyveromyces lactis, Kluyveromyces marxianus, Aspergillus terreus, Aspergillus niger and Pichia pastoris.

Examples of algae include, but are not limited to, Chlorella strains and species selected from Chlorella minutissima, Chlorella emersonii, Chlorella sorokiniana, Chlorella ellipsoidea, Chlorella sp., or Chlorella protothecoides.

The biomass (P4HB or C4 chemical) can then be treated to produce versatile intermediates that can be further processed to yield desired commodity and specialty products.

In a seventh aspect, of any of the first, second, third, fourth, fifth, or sixth aspects of the methods or any of the first, second, third, fourth, fifth, sixth, seventh, or eighth embodiments, a recombinant engineered biomass from a host organism utilizes a renewable source for generating the C4 chemical product or 4-hydroxybutyrate homopolymer that can subsequently be converted to the useful intermediates and chemical products. In some embodiments, a source of the renewable feedstock is selected from glucose, levoglucosan, fructose, sucrose, arabinose, maltose, lactose, xylose, fatty acids, vegetable oils, biomass-derived synthesis gas, and methane originating from landfill gas, or a combination of two or more of these.

In an eighth aspect of any of the first, second, third, fourth, fifth, sixth or seventh aspects or any of the first, second, third, fourth, fifth, sixth, seventh, or eighth embodiments, the invention further includes the controlled processing of the enriched C4 chemical product or P4HB biomass produced by the methods described herein to C4 chemicals.

The advantages of this bioprocess include the use of a renewable carbon source as the feedstock material, reduction of input energy needed to produce the product by an alternative method, lower greenhouse emissions and the production of a C4 chemical product or P4HB at increased yields without adverse toxicity effects to the host cell (which could limit process efficiency).

In a ninth aspect of any of the first, second, third, fourth, fifth, sixth, seventh or eighth aspects or any of the first, second, third, fourth, fifth, sixth, seventh, or eighth embodiments, the genetically engineered biomass for use in the processes of the invention is from a recombinant host having a poly-4-hydroxybutyrate or C4 chemical product pathway and stably expressing two or more genes encoding two or more enzymes, three or more genes encoding three or more enzymes, four of more genes encoding four or more enzymes, five or more genes encoding five or more enzymes, or six or more genes encoding six or more enzymes selected from alpha-ketoglutarate decarboxylase, wherein the alpha-ketoglutarate decarboxylase converts alpha-ketoglutarate to succinate semialdehyde, a 2-oxoglutaratedecarboxylase enzyme, wherein the 2-oxoglutaratedecarboxylase enzyme converts alpha-ketoglutarate to succinate semialdehyde, a phosphoenolpyruvate carboxylase wherein the phosphoenolpyruvate carboxylase converts phosphoenol pyruvate to oxaloacetate; and optionally having a disruption in one or more genes (or reduction in the expression of the gene product), two or more genes, three or more genes, or four genes selected from yneI, gabD, astD, pykF, pykA and SucCD.

In a tenth aspect, a genetically modified organism having a modified poly-4-hydroxybutyrate pathway wherein the production of poly-4-hydroxybutyrate is increased by incorporating or more genes that are stably expressed encode one or more enzymes selected from: alpha-ketoglutarate decarboxylase, 2-oxoglutaratedecarboxylase, malonyl-CoA reductase, NADH-dependent fumarate reductase, oxidative stress-resistant 1,2 propanediol oxidoreducatase, pyruvate carboxylase and NADH kinase is described.

In a first embodiment of the tenth aspect, the one or more enzyme is selected from: an alpha-ketoglutarate decarboxylase from Pseudonocardia dioxanivorans or mutants and homologues thereof; an 2-oxoglutaratedecarboxylase enzyme from Synechococcus sp. PCC 7002 or mutants and homologues thereof; a malonyl-CoA reductase from Metallosphaera sedula or mutants and homologues thereof; a malonyl-CoA reductase from Sulfolous tokodaii or mutants and homologues thereof; an oxidative stress-resistant 1,2 propanediol oxidoreducatase from E. coli, mutants and homologues thereof; an NADH-dependent fumarate reductase from Trypanosoma brucei, mutants and homologues thereof; a pyruvate carboxylase from L. lactis, mutants and homologues thereof and an NADH kinase from Aspergillus nidulans, mutants and homologues thereof.

In a second embodiment of the tenth aspect and its first embodiment, the organism further has a stably incorporated gene encoding a succinate semialdehyde dehydrogenase converts succinyl-CoA to succinic semialdehyde, for example from Clostridium kluyveri or homologues thereof.

In a third embodiment of the tenth aspect, or of the first or second embodiment of the tenth aspect, the genetically engineered organism having a poly-4-hydroxybutyrate pathway has an inhibiting mutation in its CoA-independent NAD-dependent succinic semialdehyde dehydrogenase gene or its CoA-independent NADP-dependent succinic semialdehyde dehydrogenase gene, or having inhibiting mutations in both genes, and having stably incorporated one or more genes encoding one or more enzymes selected from a succinate semialdehyde dehydrogenase wherein the succinate semialdehyde dehydrogenase converts succinyl-CoA to succinic semialdehyde, a succinic semialdehyde reductase wherein the succinic semialdehyde reductase converts succinic semialdehyde to 4-hydroxybutyrate, a CoA transferase wherein the CoA transferase converts 4-hydroxybutyrate to 4-hydroxybutyryl-CoA, and a polyhydroxyalkanoate synthase wherein the polyhydroxyalkanoate synthase polymerizes 4-hydroxybutyryl-CoA to poly-4-hydroxybutyrate.

In a fourth embodiment of the tenth aspect or of the first, second or third embodiment of the tenth aspect, the organism has a disruption (or a reduction in the expression of the gene product) in one or more genes selected from yneI, gabD, pykF, pykA, astD and sucCD.

In certain aspects, one or more nucleic acids can comprise a “one gene family” and encode a single heteromeric enzyme (e.g., sucAB1pdA is three genes that encode one enzyme) such circumstances are contemplated in the meaning on one or more genes encoding one or more enzymes.

In certain embodiments of the invention, the biomass (P4HB or C4 chemical product) is treated to produce desired chemicals. In a certain embodiment, the biomass is heated or pyrolysed to produce the chemicals from the P4HB biomass. The heating is at a temperature of about 100° C. to about 350° C. or about 200° C. to about 350° C., or from about 225° C. to 300° C. In some embodiments, the heating reduces the water content of the biomass to about 5 wt %, or less.

In some embodiments, C4 chemicals and their derivatives are produced from the methods described herein. For example, gamma-butyrolactone (GBL) can be produced by heat and enzymatic treatment that may further be processed for production of other desired commodity and specialty products, for example 1,4-butanediol (BDO), tetrahydrofuran (THF), N-methylpyrrolidone (NMP), N-ethylpyrrolidone (NEP), 2-pyrrolidinone, N-vinylpyrrolidone (NVP), polyvinylpyrrolidone (PVP) and the like. Others include succinic acid, 1,4-butanediamide, succinonitrile, succinamide, and 2-pyrrolidone (2-Py).

Additionally, the expended (residual) PHA reduced biomass is further utilized for energy development, for example as a fuel to generate process steam and/or heat.

### BRIEF DESCRIPTION OF THE DRAWINGS

The foregoing will be apparent from the following more particular description of example embodiments of the invention, as illustrated in the accompanying drawings.

FIG. 1 is a schematic diagram of exemplary E. coli central metabolic pathways showing reactions that were modified or introduced in the Examples or that could be modified in the future. Reactions that were eliminated by deleting the corresponding genes in certain Examples are marked with an “X”. Abbreviations: “PEP”, phosphoenolpyruvate; “PYR”, pyruvate; “AcCoA”, acetyl-CoA; “CIT”, citrate; “ICT”, isocitrate; “αKG”, alpha-ketoglutarate; “SUC-CoA”, succinyl-CoA; “SUC”, succinate; “Fum”, fumarate; “MAL”, malate; “OAA”, oxaloacetate; “SSA”, succinic semialdehyde; “4HB”, 4-hydroxybutyrate; “4HB-CoA”, 4-hydroxybutyryl-CoA; “4HB-P”, 4-hydroxybutyryl-phosphate; “P4HB”, poly-4-hydroxybutyrate; “GOx”, glyoxalate; “CoA”, coenzyme A; “PAN”, pantothenate. Numbered reactions: “1”, pyruvate kinase; “2”, phosphoenolpyruvate carboxylase; “3”, pyruvate carboxylase; “4”, alpha-ketoglutarate dehydrogenase; “5”, succinate semialdehyde dehydrogenase; “6”, alpha-ketoglutarate decarboxylase, also known as 2-oxoglutarate decarboxylase; “7”, succinate semialdehyde dehydrogenase (NAD⁺- and NADP⁺-dependent); “8”, succinic semialdehyde reductase; “9”, CoA transferase; “10”, butyrate kinase; “11”, phosphotransbutyrylase; “12”, polyhydroxyalkanoate synthase; “13”, succinyl-CoA synthetase; “14”, succinate dehydrogenase or fumarate reductase (menaquinol- and NADH-dependent); “15”, isocitrate lyase; “16”, malate synthase A; “17”, NADH kinase.

FIG. 2 is a phylogenetic tree showing KgdM homologues of Mycobacterium tuberculosis. The homologues whose genes were selected for cloning and recombinant expression in P4HB production strains are underlined and indicated by numbers: kgdM_MBLX from M. tuberculosis (1), sucA from M. bovis (2), M. smegmatis (3), Dietzia cinnamea (4), Pseudonocardia dioxanivorans (5), and Corynebacterium aurimucosum (6).

### DETAILED DESCRIPTION OF THE INVENTION

A description of example embodiments of the invention follows.

The present invention provides methods of increasing the production of a 4-carbon (C4) product or a polymer of 4-carbon monomers from a renewable feedstock, comprising providing a genetically modified organism having a reduced activity of alpha-ketoglutarate dehydrogenase such that growth is impaired as compared to a wild-type organism without the reduced activity; and providing one or more genes that are stably expressed that encodes an enzyme with an activity catalyzing the decarboxylation of alpha-ketoglutarate to succinic semialdehyde; wherein growth is improved and the carbon flux from the renewable feedstock 4-carbon (C4) product or a polymer of 4-carbon monomers is increased.

Also included are methods of increasing production of a 4-carbon (C4) product in a genetically modified organism (recombinant host) having a C4 pathway by stable expression of a gene encoding an enzyme that catalyzes the decarboxylation of alpha-ketoglutarate to succinic semialdehyde for producing the C4 product. The organism has a deletion of the alpha-ketoglutate dehydrogenase (sucAB) gene. This pathway provides increased yield of desired products that can be cultured using renewable feedstocks in quantities that are a viable, cost effective alternative to petroleum based products.

In certain embodiments, the 4-carbon product produced by the methods include but are not limited to 1,4-butanediol, 4-hydroxybutyrate, gamma-butyrolactone, tetrahydrofuran, N-methylpyrrolidone, N-ethylpyrrolidone, 2-pyrrolidinone, N-vinylpyrrolidone, polyvinylpyrrolidone, succinic acid, 1,4-butanediamide, succinonitrile, succinamide and 2-pyrrolidone (2-Py).

The present invention provides methods for producing genetically engineered organisms (e.g., recombinant hosts) that have been modified to produce increased amounts of biobased poly-4-hydroxybutyrate (P4HB), 4-carbon (C4) product or a polymer of 4-carbon monomers by stably incorporating genes into the host organism to modify the P4HB, 4-carbon (C4) product or polymer of 4-carbon monomers' metabolic pathway. Also described herein is the biobased biomass produced by improved production processes using the recombinant host organisms described herein.

These recombinant hosts have been genetically constructed to increase the yield of P4HB, 4-carbon (C4) product or a polymer of 4-carbon monomers by manipulating (e.g., inhibiting and/or overexpressing) certain genes in the P4HB, 4-carbon (C4) product or a polymer of 4-carbon monomers' pathway to increase the yield of P4HB, 4-carbon (C4) product or a polymer of 4-carbon monomers in the biomass. The biomass is produced in a fermentation process in which the genetically engineered microbe is fed a renewable substrate. Renewable substrates include fermentation feedstocks such as sugars, levoglucosan, vegetable oils, fatty acids or synthesis gas produced from plant crop materials. The level of P4HB, 4-carbon (C4) product or a polymer of 4-carbon monomers produced in the biomass from the renewable substrate is greater than 5% (e.g., about 10%, about 20%, about 30%, about 40%, about 50%, about 60%, about 70%, about 80%) of the total dry weight of the biomass. The biomass is then available for post purification and modification methodologies to produce other biobased C4 chemicals and derivatives.

Genetically-modified biomass systems have been developed which produce a wide variety of biodegradable PHA polymers and copolymers in high yield (Lee (1996), Biotechnology & Bioengineering 49:1-14; Braunegg et al. (1998), J. Biotechnology 65:127-161; Madison, L. L. and Huisman, G. W. (1999), Metabolic Engineering of Poly-3-Hydroxyalkanoates; From DNA to Plastic, in: Microbiol. Mol. Biol. Rev. 63:21-53). PHA polymers are well known to be thermally unstable compounds that readily degrade when heated up to and beyond their melting points (Cornelissen et al., Fuel, 87, 2523, 2008). This is usually a limiting factor when processing the polymers for plastic applications that can, however, be leveraged to create biobased, chemical manufacturing processes starting from 100% renewable resources.

Recombinant Hosts with Metabolic Pathways for Producing P4HB

Genetic engineering of hosts (e.g., bacteria, fungi, algae, plants and the like) as production platforms for modified and new materials provides a sustainable solution for high value eco-friendly industrial applications for production of chemicals. The processes described herein avoid toxic effects to the host organism by producing the biobased chemical post culture or post harvesting, are cost effective and highly efficient (e.g., use less energy to make), decrease greenhouse gas emissions, use renewable resources and can be further processed to produce high purity products from C4 products in high yield.

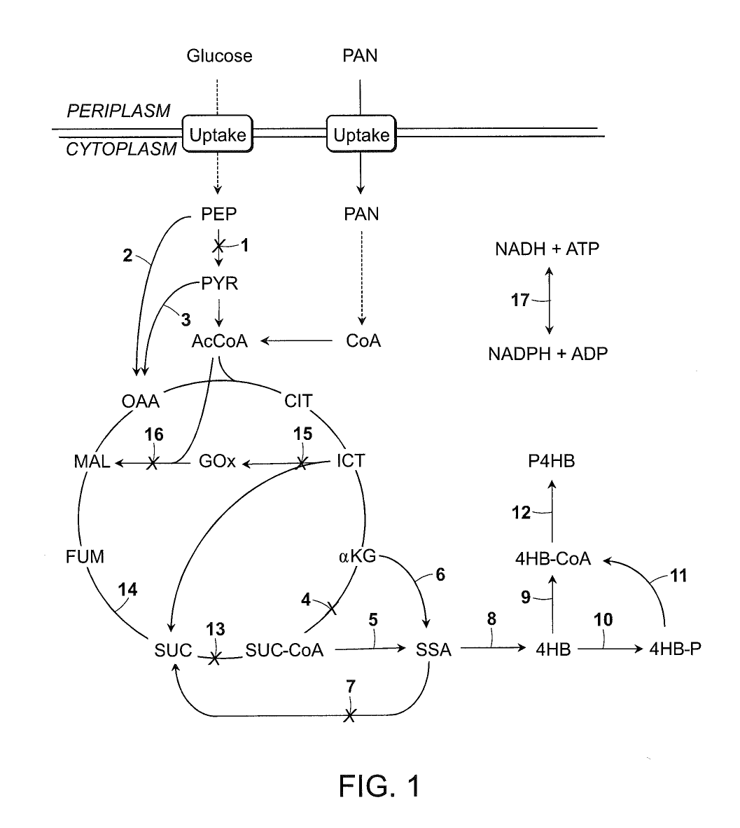

The PHA biomass utilized in the methods described herein is genetically engineered to produce increased amounts of poly-4-hydroxybutyrate (P4HB) over the un-optimized genetically engineered P4HB pathway. An exemplary pathway for production of P4HB is provided in FIG. 1 and a more detailed description of the pathway, recombinant hosts that produce P4HB biomass is provided below. The pathway can be engineered to increase production of P4HB from carbon feed sources.

As used herein, “P4HB biomass” is intended to mean any genetically engineered biomass from a recombinant host (e.g., bacteria,) that includes a non-naturally occurring amount of the polyhydroxyalkanoate polymer e.g., poly-4-hydroxybutyrate (P4HB). In some embodiments, a source of the P4HB biomass is bacteria, yeast, fungi, algae, plant crop, cyanobacteria, or a mixture of any two or more thereof. In certain embodiments, the biomass titer (g/L) of P4HB has been increased when compared to the host without the overexpression or inhibition of one or more genes in the P4HB pathway. In certain embodiments, the P4HB titer is reported as a percent dry cell weight (% dcw) or as grams of P4HB/Kg biomass.

As used herein, “C4 chemical product biomass” is intended to mean any genetically engineered biomass from a recombinant host (e.g., bacteria,) that includes a non-naturally occurring amount of a C4 chemical product made by a C4 pathway (e.g., BDO made by a BDO pathway). In some embodiments, a source of the C4 chemical product biomass is bacteria, yeast, fungi, algae, plant crop cyanobacteria, or a mixture of any two or more thereof. In certain embodiments, the biomass titer (g/L) of C4 chemical product has been increased when compared to the host without the overexpression or inhibition of one or more genes in the C4 chemical pathway. In certain embodiments, the C4 chemical product titer is reported as a percent dry cell weight (% dcw) or as grams of C4 chemical product titer/Kg biomass.

“Overexpression” refers to the expression of a polypeptide or protein encoded by a DNA introduced into a host cell, wherein the polypeptide or protein is either not normally present in the host cell, or where the polypeptide or protein is present in the host cell at a higher level than that normally expressed from the endogenous gene encoding the polypeptide or protein. “Inhibition” or “down regulation” refers to the suppression or deletion of a gene that encodes a polypeptide or protein. In some embodiments, inhibition means inactivating the gene that produces an enzyme in the pathway. In certain embodiments, the genes introduced are from a heterologous organism.

Genetically engineered microbial PHA production systems with fast growing hosts such as Escherichia coli have been developed. In certain embodiments, genetic engineering also allows for the modification of wild-type microbes to improve the production of the P4HB polymer. Examples of PHA production modifications are described in Steinbuchel & Valentin, FEMS Microbiol. Lett. 128:219-28 (1995). PCT Publication No. WO 98/04713 describes methods for controlling the molecular weight using genetic engineering to control the level of the PHA synthase enzyme. Commercially useful strains, including Alcaligenes eutrophus (renamed as Ralstonia eutropha or Cupriavidus necator), Alcaligenes latus (renamed also as Azohydromonas lata), Azotobacter vinlandii, and Pseudomonads, for producing PHAs are disclosed in Lee, Biotechnology & Bioengineering, 49:1-14 (1996) and Braunegg et al., (1998), J. Biotechnology 65: 127-161. U.S. Pat. Nos. 6,316,262; 7,229,804; 6,759,219 and 6,689,589 describe biological systems for manufacture of PHA polymers containing 4-hydroxyacids, incorporated by reference herein.

Although there have been reports of producing 4-hydroxybutyrate copolymers from renewable resources such as sugar or amino acids, the level of 4HB in the copolymers produced from scalable renewable substrates has been much less than 50% of the monomers in the copolymers and therefore unsuitable for practicing the disclosed invention. Production of the P4HB biomass or C4 chemical product biomass using an engineered microorganism with renewable resources where the level of P4HB or C4 chemical product in the biomass is sufficient to practice the disclosed invention (i.e., greater than 40%, 50%, 60% or 65% of the total biomass dry weight) has not previously been achieved.

The weight percent PHA in the wild-type biomass varies with respect to the source of the biomass. For microbial systems produced by a fermentation process from renewable resource-based feedstocks such as sugars, levoglucosan, vegetable oils or glycerol, the amount of PHA in the wild-type biomass may be about 65 wt %, or more, of the total weight of the biomass. For plant crop systems, in particular biomass crops such as sugarcane or switchgrass, the amount of PHA may be about 3%, or more, of the total weight of the biomass. For algae or cyanobacteria) systems, the amount of PHA may be about 40%, or more of the total weight of the biomass.

In certain aspects of the invention, the recombinant host has been genetically engineered to produce an increased amount of C4 chemical product as compared to the wild-type host. The wild-type C4 chemical product biomass refers to the amount of C4 chemical product that an organism typically produces in nature.

For example, in certain embodiments, the P4HB or C4 chemical product is increased between about 20% to about 90% over the control or between about 50% to about 80%. In other embodiments, the recombinant host produces at least about a 20% increase of P4HB over control strain, at least about a 30% increase over control, at least about a 40% increase over control, at least about a 50% increase over control, at least about a 60% increase over control, at least about a 70% increase over control, at least about a 75% increase over control, at least about a 80% increase over control, or at least about a 90% increase over control. In other embodiments, the C4 chemical product is between about a 2-fold increase to about a 400% or 4-fold increase over the amount produced by the wild-type host. The amount of C4 chemical product in the host or plant is determined by gas chromatography according to procedures described in Doi, Microbial Polyesters, John Wiley&Sons, p24, 1990. In certain embodiments, a biomass titer of 100-120 g P4HB/Kg of biomass can be achieved. In other embodiments, the amount of P4HB titer is presented as percent dry cell weight (% dcw).

In certain aspects of the invention, the recombinant host has been genetically engineered to produce an increased amount of C4 chemical product as compared to the wild-type host. The wild-type C4 chemical product biomass refers to the amount of C4 chemical product that an organism typically produces in nature.

Producing C4 Chemicals from the P4HB Biomass

In general, during or following production (e.g., culturing) of the P4HB or C4 chemical product biomass, the biomass is combined with a catalyst under suitable conditions to help convert the P4HB polymer or C4 chemical product to a C4 product (e.g., gamma-butyrolactone). The catalyst (in solid or solution form) and biomass are combined for example by mixing, flocculation, centrifuging or spray drying, or other suitable method known in the art for promoting the interaction of the biomass and catalyst driving an efficient and specific conversion of P4HB to gamma-butyrolactone. In some embodiments, the biomass is initially dried, for example at a temperature between about 100° C. and about 150° C. and for an amount of time to reduce the water content of the biomass. The dried biomass is then re-suspended in water prior to combining with the catalyst. Suitable temperatures and duration for drying are determined for product purity and yield and can in some embodiments include low temperatures for removing water (such as between 25° C. and 150° C.) for an extended period of time or in other embodiments can include drying at a high temperature (e.g., above 450° C.) for a short duration of time. Under “suitable conditions” refers to conditions that promote the catalytic reaction. For example, under conditions that maximize the generation of the product such as in the presence of co-agents or other material that contributes to the reaction efficiency. Other suitable conditions include in the absence of impurities, such as metals or other materials that would hinder the reaction from progressing.

As used herein, “catalyst” refers to a substance that initiates or accelerates a chemical reaction without itself being affected or consumed in the reaction. Examples of useful catalysts include metal catalysts. In certain embodiments, the catalyst lowers the temperature for initiation of thermal decomposition and increases the rate of thermal decomposition at certain pyrolysis temperatures (e.g., about 200° C. to about 325° C.).

In some embodiments, the catalyst is a chloride, oxide, hydroxide, nitrate, phosphate, sulphonate, carbonate or stearate compound containing a metal ion. Examples of suitable metal ions include aluminum, antimony, barium, bismuth, cadmium, calcium, cerium, chromium, cobalt, copper, gallium, iron, lanthanum, lead, lithium, magnesium, molybdenum, nickel, palladium, potassium, silver, sodium, strontium, tin, tungsten, vanadium or zinc and the like. In some embodiments, the catalyst is an organic catalyst that is an amine, azide, enol, glycol, quaternary ammonium salt, phenoxide, cyanate, thiocyanate, dialkyl amide and alkyl thiolate. In some embodiments, the catalyst is calcium hydroxide. In other embodiments, the catalyst is sodium carbonate. Mixtures of two or more catalysts are also included.

In certain embodiments, the amount of metal catalyst is about 0.1% to about 15% or about 1% to about 25%, or about 4% to about 50% based on the weight of metal ion relative to the dry solid weight of the biomass. In some embodiments, the amount of catalyst is between about 7.5% and about 12%. In other embodiments, the amount of catalyst is about 0.5% dry cell weight, about 1%, about 2%, about 3%, about 4%, about 5, about 6%, about 7%, about 8%, about 9%, or about 10%, or about 11%, or about 12%, or about 13%, or about 14%, or about 15%, or about 20%, or about 30%, or about 40% or about 50% or amounts in between these.

As used herein, the term “sufficient amount” when used in reference to a chemical reagent in a reaction is intended to mean a quantity of the reference reagent that can meet the demands of the chemical reaction and the desired purity of the product.

### Thermal Degradation of the P4HB Biomass to C4 Products

“Heating,” “pyrolysis”, “thermolysis” and “torrefying” as used herein refer to thermal degradation (e.g., decomposition) of the P4HB biomass for conversion to C4 products. In general, the thermal degradation of the P4HB biomass occurs at an elevated temperature in the presence of a catalyst. For example, in certain embodiments, the heating temperature for the processes described herein is between about 200° C. to about 400° C. In some embodiments, the heating temperature is about 200° C. to about 350° C. In other embodiments, the heating temperature is about 300° C. “Pyrolysis” typically refers to a thermochemical decomposition of the biomass at elevated temperatures over a period of time. The duration can range from a few seconds to hours. In certain conditions, pyrolysis occurs in the absence of oxygen or in the presence of a limited amount of oxygen to avoid oxygenation. The processes for P4HB biomass pyrolysis can include direct heat transfer or indirect heat transfer. “Flash pyrolysis” refers to quickly heating the biomass at a high temperature for fast decomposition of the P4HB biomass, for example, depolymerization of a P4HB in the biomass. Another example of flash pyrolysis is RTP™ rapid thermal pyrolysis. RTP™ technology and equipment from Envergent Technologies, Des Plaines, Ill. converts feedstocks into bio-oil. “Torrefying” refers to the process of torrefaction, which is an art-recognized term that refers to the drying of biomass. The process typically involves heating a biomass in a temperature range from 200-350° C., over a relatively long duration (e.g., 10-30 minutes), typically in the absence of oxygen. The process results for example, in a torrefied biomass having a water content that is less than 7 wt % of the biomass. The torrefied biomass may then be processed further. In some embodiments, the heating is done in a vacuum, at atmospheric pressure or under controlled pressure. In certain embodiments, the heating is accomplished without the use or with a reduced use of petroleum generated energy.

In certain embodiments, the P4HB biomass is dried prior to heating. Alternatively, in other embodiments, drying is done during the thermal degradation (e.g., heating, pyrolysis or torrefaction) of the P4HB biomass. Drying reduces the water content of the biomass. In certain embodiments, the biomass is dried at a temperature of between about 100° C. to about 350° C., for example, between about 200° C. and about 275° C. In some embodiments, the dried 4PHB biomass has a water content of 5 wt %, or less.

In certain embodiments, the heating of the P4HB biomass/catalyst mixture is carried out for a sufficient time to efficiently and specifically convert the P4HB biomass to a C4 product. In certain embodiments, the time period for heating is from about 30 seconds to about 1 minute, from about 30 seconds to about 1.5 minutes, from about 1 minute to about 10 minutes, from about 1 minute to about 5 minutes or a time between, for example, about 1 minute, about 2 minutes, about 1.5 minutes, about 2.5 minutes, about 3.5 minutes.

In other embodiments, the time period is from about 1 minute to about 2 minutes. In still other embodiments, the heating time duration is for a time between about 5 minutes and about 30 minutes, between about 30 minutes and about 2 hours, or between about 2 hours and about 10 hours or for greater that 10 hours (e.g., 24 hours).

In certain embodiments, the heating temperature is at a temperature of about 200° C. to about 350° C. including a temperature between, for example, about 205° C., about 210° C., about 215° C., about 220° C., about 225° C., about 230° C., about 235° C., about 240° C., about 245° C., about 250° C., about 255° C. about 260° C., about 270° C., about 275° C., about 280° C., about 290° C., about 300° C., about 310° C., about 320° C., about 330° C., about 340° C., or 345° C. In certain embodiments, the temperature is about 250° C. In certain embodiments, the temperature is about 275° C. In other embodiments, the temperature is about 300° C.

In certain embodiments, the process also includes flash pyrolyzing the residual biomass for example at a temperature of 500° C. or greater for a time period sufficient to decompose at least a portion of the residual biomass into pyrolysis liquids. In certain embodiments, the flash pyrolyzing is conducted at a temperature of 500° C. to 750° C. In some embodiments, a residence time of the residual biomass in the flash pyrolyzing is from 1 second to 15 seconds, or from 1 second to 5 seconds or for a sufficient time to pyrolyze the biomass to generate the desired pyrolysis precuts, for example, pyrolysis liquids. In some embodiments, the flash pyrolysis can take place instead of torrefaction. In other embodiments, the flash pyrolysis can take place after the torrrefication process is complete.

As used herein, “pyrolysis liquids” are defined as a low viscosity fluid with up to 15-20% water, typically containing sugars, aldehydes, furans, ketones, alcohols, carboxylic acids and lignins. Also known as bio-oil, this material is produced by pyrolysis, typically fast pyrolysis of biomass at a temperature that is sufficient to decompose at least a portion of the biomass into recoverable gases and liquids that may solidify on standing. In some embodiments, the temperature that is sufficient to decompose the biomass is a temperature between 400° C. to 800° C.

In certain embodiments, “recovering” the C4 product vapor includes condensing the vapor. As used herein, the term “recovering” as it applies to the vapor means to isolate it from the P4HB biomass materials, for example including but not limited to: recovering by condensation, separation methodologies, such as the use of membranes, gas (e.g., vapor) phase separation, such as distillation, and the like. Thus, the recovering may be accomplished via a condensation mechanism that captures the monomer component vapor, condenses the monomer component vapor to a liquid form and transfers it away from the biomass materials.

As a non-limiting example, the condensing of the vapor may be described as follows. The incoming gas/vapor stream from the pyrolysis/torrefaction chamber enters an interchanger, where the gas/vapor stream may be pre-cooled. The gas/vapor stream then passes through a chiller where the temperature of the gas/vapor stream is lowered to that required to condense the designated vapors from the gas by indirect contact with a refrigerant. The gas and condensed vapors flow from the chiller into a separator, where the condensed vapors are collected in the bottom. The gas, free of the vapors, flows from the separator, passes through the Interchanger and exits the unit. The recovered liquids flow, or are pumped, from the bottom of the separator to storage. For some of the products, the condensed vapors solidify and the solid is collected.

In certain embodiments, recovery of the catalyst is further included in the processes of the invention. For example, when a calcium catalyst is used calcination is a useful recovery technique. Calcination is a thermal treatment process that is carried out on minerals, metals or ores to change the materials through decarboxylation, dehydration, devolatilization of organic matter, phase transformation or oxidation. The process is normally carried out in reactors such as hearth furnaces, shaft furnaces, rotary kilns or more recently fluidized beds reactors. The calcination temperature is chosen to be below the melting point of the substrate but above its decomposition or phase transition temperature. Often this is taken as the temperature at which the Gibbs free energy of reaction is equal to zero. For the decomposition of CaCO₃ to CaO, the calcination temperature at ΔG=0 is calculated to be ˜850° C. Typically for most minerals, the calcination temperature is in the range of 800-1000° C.

To recover the calcium catalyst from the biomass after recovery of the C4 product, one would transfer the spent biomass residue directly from pyrolysis or torrefaction into a calcining reactor and continue heating the biomass residue in air to 825-850° C. for a period of time to remove all traces of the organic biomass. Once the organic biomass is removed, the catalyst could be used as is or purified further by separating the metal oxides present (from the fermentation media and catalyst) based on density using equipment known to those in the art.

In other embodiments, the product can be further purified if needed by additional methods known in the art, for example, by distillation, by reactive distillation by treatment with activated carbon for removal of color and/or odor bodies, by ion exchange treatment, by liquid-liquid extraction—with an immiscible solvent to remove fatty acids etc, for purification after recovery, by vacuum distillation, by extraction distillation or using similar methods that would result in further purifying product to increase the yield of product. Combinations of these treatments can also be utilized.

As used herein, the term “residual biomass” refers to the biomass after PHA conversion to the small molecule intermediates. The residual biomass may then be converted via torrefaction to a useable, fuel, thereby reducing the waste from PHA production and gaining additional valuable commodity chemicals from typical torrefaction processes. The torrefaction is conducted at a temperature that is sufficient to densify the residual biomass. In certain embodiments, processes described herein are integrated with a torrefaction process where the residual biomass continues to be thermally treated once the volatile chemical intermediates have been released to provide a fuel material. Fuel materials produced by this process are used for direct combustion or further treated to produce pyrolysis liquids or syngas. Overall, the process has the added advantage that the residual biomass is converted to a higher value fuel which can then be used for the production of electricity and steam to provide energy for the process thereby eliminating the need for waste treatment.

A “carbon footprint” is a measure of the impact the processes have on the environment, and in particular climate change. It relates to the amount of greenhouse gases produced.

In certain embodiments, it may be desirable to label the constituents of the biomass. For example, it may be useful to deliberately label with an isotope of carbon (e.g., ¹³C) to facilitate structure determination or for other means. This is achieved by growing microorganisms genetically engineered to express the constituents, e.g., polymers, but instead of the usual media, the bacteria are grown on a growth medium with ¹³C-containing carbon source, such as glucose, pyruvic acid, etc. In this way polymers can be produced that are labeled with ¹³C uniformly, partially, or at specific sites. Additionally, labeling allows the exact percentage in bioplastics that came from renewable sources (e.g., plant derivatives) can be known via ASTM D6866—an industrial application of radiocarbon dating. ASTM D6866 measures the Carbon 14 content of biobased materials; and since fossil-based materials no longer have Carbon 14, ASTM D6866 can effectively dispel inaccurate claims of biobased content

### EXAMPLES

The present technology is further illustrated by the following examples, which should not be construed as limiting in any way.

These examples describe a number of biotechnology tools and methods for the construction of strains that generate a product of interest. Suitable host strains, the potential source and a list of recombinant genes used in these examples, suitable extrachromosomal vectors, suitable strategies and regulatory elements to control recombinant gene expression, and a selection of construction techniques to overexpress genes in or inactivate genes from host organisms are described. These biotechnology tools and methods are well known to those skilled in the art.

### Suitable Host Strains

In certain embodiments, the host strain is E. coli K-12 strain LS5218 (Spratt et al., J. Bacteriol. 146 (3):1166-1169 (1981); Jenkins and Nunn, J. Bacteriol. 169 (1):42-52 (1987)) or strain MG1655 (Guyer et al., Cold Spr. Harb. Symp. Quant. Biol. 45:135-140 (1981)). Other suitable E. coli K-12 host strains include, but are not limited to, WG1 and W3110 (Bachmann Bacteriol. Rev. 36(4):525-57 (1972)). Alternatively, E. coli strain W (Archer et al., BMC Genomics 2011, 12:9 doi:10.1186/1471-2164-12-9) or E. coli strain B (Delbruck and Luria, Arch. Biochem. 1:111-141 (1946)) and their derivatives such as REL606 (Lenski et al., Am. Nat. 138:1315-1341 (1991)) are other suitable E. coli host strains.

Other exemplary microbial host strains include but are not limited to: Ralstonia eutropha, Zoogloea ramigera, Allochromatium vinosum, Rhodococcus ruber, Delftia acidovorans, Aeromonas caviae, Synechocystis sp. PCC 6803, Synechococcus elongatus PCC 7942, Thiocapsa pfenigii, Bacillus megaterium, Acinetobacter baumannii, Acinetobacter baylyi, Clostridium kluyveri, Methylobacterium extorquens, Nocardia corralina, Nocardia salmonicolor, Pseudomonas fluorescens, Pseudomonas oleovorans, Pseudomonas sp. 6-19, Pseudomonas sp. 61-3 and Pseudomonas putida, Rhodobacter sphaeroides, Alcaligenes latus, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, and Clostridium acetobutylicum. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger and Pichia pastoris.

Exemplary algal strains include but are not limited to: Chlorella strains, species selected from: Chlorella minutissima, Chlorella emersonii, Chlorella sorokiniana, Chlorella ellipsoidea, Chlorella sp., or Chlorella protothecoides.

### Source of Recombinant Genes

Sources of encoding nucleic acids for a P4HB pathway enzyme can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. Exemplary species for such sources include, for example, Escherichia coli, Saccharomyces cerevisiae, Saccharomyces kluyveri, Synechocystis sp. PCC 6803, Synechococcus elongatus PCC 7942, Synechococcus sp. PCC 7002, Chlorogleopsis sp. PCC 6912, Chloroflexus aurantiacus, Clostridium kluyveri, Clostridium acetobutylicum, Clostridium beijerinckii, Clostridium saccharoperbutylacetonicum, Clostridium perjringens, Clostridium difficile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Chlorella minutissima, Chlorella emersonii, Chlorella sorokiniana, Chlorella ellipsoidea, Chlorella sp., Chlorella protothecoides, Homo sapiens, Oryctolagus cuniculus, Rhodobacter spaeroides, Thermoanaerobacter brockii, Metallosphaera sedula, Leuconostoc mesenteroides, Roseiflexus castenholzii, Erythrobacter, Simmondsia chinensis, Acinetobacter species, including Acinetobacter calcoaceticus and Acinetobacter baylyi, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Rattus norvegicus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Sus scrofa, Caenorhabditis elegans, Corynebacterium glutamicum, Acidaminococcus fermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilus, Campylobacter jejuni, Haemophilus influenzae, Serratia marcescens, Citrobacter amalonaticus, Myxococcus xanthus, Fusobacterium nuleatum, Penicillium chrysogenum, marine gamma proteobacterium, butyrate-producing bacterium, and Trypanosoma brucei. For example, microbial hosts (e.g., organisms) having P4HB biosynthetic production are exemplified herein with reference to an E. coli host. However, with the complete genome sequence available for now more than 550 species (with more than half of these available on public databases such as the NCBI), including 395 microorganism genomes and a variety of yeast, fungi, plant, and mammalian genomes, the identification of genes encoding the requisite P4HB biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations enabling biosynthesis of P4HB and other compounds of the invention described herein with reference to a particular organism such as E. coli can be readily applied to other microorganisms, including prokaryotic and eukaryotic organisms alike. Given the teachings and guidance provided herein, those skilled in the art will know that a metabolic alteration exemplified in one organism can be applied equally to other organisms.

### Production of Transgenic Host for Producing 4HB

Transgenic (recombinant) hosts for producing P4HB are genetically engineered using conventional techniques known in the art. The genes cloned and/or assessed for host strains producing 4HB-containing PHA and 4-carbon chemicals are presented below in Table 1A, along with the appropriate Enzyme Commission number (EC number) and references. Some genes were synthesized for codon optimization while others were cloned via PCR from the genomic DNA of the native or wild-type host. As used herein, “heterologous” means from another host. The host can be the same or different species. FIG. 1 is an exemplary pathway for producing P4HB.

TABLE 1A
Genes overproduced or deleted in microbial host strains producing 4HB-containing
PHA and 4-carbon chemicals. A star (*) after the gene name denotes that the
nucleotide sequence was optimized for expression in E. coli.

| Reaction | | | | |
|---|---|---|---|---|
| number | | | | |
| (FIG. 1) | Gene Name | Enzyme Name | EC Number | Accession No. |
| 1 | pykF | Pyruvate kinase I | 2.7.1.40 | NP_416191 |
| 1 | pykA | Pyruvate kinase II | 2.7.1.40 | NP_416368 |
| 2 | ppc | Phosphoenolpyruvate | 4.1.1.31 | NP_418391 |
| | | carboxylase | | |
| 3 | pyc_{L1} | Pyruvate carboxylase | 6.4.1.1 | Gene/Protein ID 1 |
| 4 | sucAB lpdA | Alpha-ketoglutarate | 1.2.4.2 | NP_415254 |
| | | dehydrogenase | (sucA) | NP_415255 |
| | | | 2.3.1.61 | NP_414658 |
| | | | (sucB) | |
| | | | 1.8.1.4 | |
| | | | (lpdA) | |
| 5 | sucD_{Ck}* | Succinate | 1.2.1.76 | WO 2011/100601 |
| | | semialdehyde | | |
| | | dehydrogenase | | |
| 5 | mcr_{St}* | Malonyl-CoA | 1.2.1.n | Gene/Protein ID 2 |
| | | reductase | | |
| 6 | kgdM | Alpha-ketoglutarate | 4.1.1.71 | NP_335730 |
| | | decarboxylase | | |
| 6 | kgdP | Alpha-ketoglutarate | 4.1.1.n | YP_004335105 |
| | | decarboxylase | | |
| 6 | kgdP-M38 | Alpha-ketoglutarate | 4.1.1.n | Gene/Protein ID 3 |
| | | decarboxylase | | |
| 6 | kgdS | 2-Oxoglutarate | 4.1.1.n | ACB00744.1 |
| | | decarboxylase | | |
| 7 | yneI | Succinate- | 1.2.1.24 | NP_416042 |
| | | semialdehyde | | |
| | | dehydrogenase, NAD+- | | |
| | | dependent | | |
| 7 | gabD | Succinate- | 1.2.1.79 | NP_417147 |
| | | semialdehyde | | |
| | | dehydrogenase, | | |
| | | NADP+-dependent | | |
| 7 | astD | Succinylglutamic | 1.2.1.— | NP_416260 |
| | | semialdehyde | | |
| | | dehydrogenase | | |
| 8 | ssaR_{At}* | Succinic | 1.1.1.61 | WO 2011/100601 |
| | | semialdehyde | | |
| | | reductase | | |
| 8 | yqhD | NADP-dependent | 1.1.1.61 | NP_417484 |
| | | aldehyde | | |
| | | dehydrogenase | | |
| 8 | yihU | Succinic semialdehyde | 1.1.1.61 | NP_418318 |
| | | reductase | | |
| 8 | fucO_{I6L-L7V} | 1,2-Propanediol | 1.1.1.77 | Gene/Protein ID 4 |
| | | oxidoreductase | | |
| | | (resistant to oxidative | | |
| | | stress) | | |
| 9 | orfZ_{Ck} | CoA transferase | 2.8.3.n | AAA92344 |
| 10 | buk1 | Butyrate kinase I | 2.7.2.7 | NP_349675 |
| 10 | buk2 | Butyrate kinase II | 2.7.2.7 | NP_348286 |
| 11 | ptb | Phosphotransbutyrylase | 2.3.1.19 | NP_349676 |
| 12 | phaC3/C1* | Polyhydroxyalkanoate | 2.3.1.n | WO 2011/100601 |
| | | synthase fusion protein | | |
| 12 | phaC183* | Polyhydroxyalkanoate | 2.3.1.n | Gene/Protein ID 5 |
| | | synthase | | |
| 12 | phaC1 | Polyhydroxyalkanoate | 2.3.1.n | YP_725940 |
| | | synthase | | |
| 13 | sucCD | Succinyl-CoA | 6.2.1.5 | NP_415256 |
| | | synthetase | | NP_415257 |
| 14 | frd_g* | Fumarate reductase | 1.3.1.6 | Gene/Protein ID 6 |
| | | (NADH-dependent) | | |
| 15 | aceA | Isocitrate lyase | 4.1.3.1 | NP_418439 |
| 16 | aceB | Malate synthase A | 2.3.3.9 | NP_418438 |
| 17 | ndk_{An}* | NADH kinase | 2.7.1.86 | XP_682106 |

Other proteins capable of catalyzing the reactions listed in Table 1A can be discovered by consulting the scientific literature, patents, BRENDA searches (http://www.brenda-enzymes.info/), and/or by BLAST searches against e.g., nucleotide or protein databases at NCBI (www.ncbi.nlm.nih.gov/). Synthetic genes can then be created to provide an easy path from sequence databases to physical DNA. Such synthetic genes are designed and fabricated from the ground up, using codons to enhance heterologous protein expression, and optimizing characteristics needed for the expression system and host. Companies such as e.g., DNA 2.0 (Menlo Park, Calif. 94025, USA) will provide such routine service. Proteins that may catalyze some of the biochemical reactions listed in Table 1A are provided in Tables 1B-1 to 1B-29.

TABLE 1B-1
Suitable homologues for the PykF and PykA proteins (pyruvate kinase,
from Escherichia coli, EC No. 2.7.1.40, which acts on
phosphoenolpyruvate to produce pyruvate and ATP; protein accession
numbers NP_416191 and NP_416368).

| Protein Name | Protein Accession No. |
|---|---|
| Pyruvate kinase | YP_725084 |
| Pyruvate kinase | XP_004056483 |
| Pyruvate kinase | XP_003385771 |
| Pyruvate kinase | XP_002491703 |
| Pyruvate kinase | NP_014992 |
| Pyruvate kinase | NP_390796 |

TABLE 1B-2
Suitable homologues for the Ppc protein (phosphoenolpyruvate
carboxylase from Escherichia coli, EC No. 4.1.1.31, which acts on
phosphoenolpyruvate and CO₂/carbonate to form oxaloacetate and
orthophosphate; protein accession number NP_418391).

| Protein Name | Protein Accession No. |
|---|---|
| Phosphoenolpyruvate carboxylase | ZP_02904134 |
| Phosphoenolpyruvate carboxylase | YP_002384844 |
| Phosphoenolpyruvate carboxylase | YP_003367228 |
| Phosphoenolpyruvate carboxylase | ZP_02345134 |
| Phosphoenolpyruvate carboxylase | ZP_04558550 |
| Phosphoenolpyruvate carboxylase | YP_003615503 |
| Phosphoenolpyruvate carboxylase | YP_002241183 |
| Phosphoenolpyruvate carboxylase | CBK84190 |
| Phosphoenolpyruvate carboxylase | YP_003208553 |

TABLE 1B-3
Suitable homologues for the Pyc_{L1} protein (pyruvate carboxylase from
Lactococcus lactis, EC 6.4.1.1, which acts on pyruvate to form
oxaloacetate; sequence as defined in Gene/Protein ID 1).

| Protein Name | Protein Accession No. |
|---|---|
| Pyruvate carboxylase | YP_471473 |
| Pyruvate carboxylase subunit A, | YP_002875552, |
| Pyruvate carboxylase subunit B | YP_002875551 |

TABLE 1B-4
Suitable homologues for the SucA protein, (E1 subunit of alpha-
ketoglutarate dehydrogenase complex from Escherichia coli which
acts on alpha-ketoglutarate to form succinyl-CoA, carbon dioxide, and
NADPH, EC No. 1.2.4.2; protein accession number NP_415254).

| Protein Name | Protein Accession No. |
|---|---|
| 2-oxoglutarate dehydrogenase | NP_001003941 |
| 2-oxoglutarate dehydrogenase | XP_003389557 |
| Kgd1p | NP_012141 |
| Component of the mitochondrial alpha- | XP_002490970 |
| ketoglutarate dehydrogenase complex | |
| 2-oxoglutarate dehydrogenase E1 component | YP_726789 |
| 2-oxoglutarate dehydrogenase E1 | NP_389819 |

TABLE 1B-5
Suitable homologues for the SucB protein, (E2 subunit of alpha-
ketoglutarate dehydrogenase complex from Escherichia coli which
acts on alpha-ketoglutarate to form succinyl-CoA, carbon dioxide, and
NADPH, EC No. 2.3.1.61; protein accession number NP_415255).

| Protein Name | Protein Accession No. |
|---|---|
| Dihydrolipoyllysine-residue succinyltransferase | NP_001231812 |
| component of 2-oxoglutarate dehydrogenase | |
| complex | |
| Dihydrolipoyllysine-residue succinyltransferase | XP_003385604 |
| component of 2-oxoglutarate dehydrogenase | |
| complex | |
| Dihydrolipoyl transsuccinylase | XP_002489434 |
| Kgd2p | NP_010432 |
| Dihydrolipoamide succinyltransferase | YP_726788 |
| Dihydrolipoamide succinyltransferase | NP_389818 |

TABLE 1B-6
Suitable homologues for the LpdA protein, (lipoamide dehydrogenase
subunit of alpha-ketoglutarate dehydrogenase complex from Escherichia
coli which acts on alpha-ketoglutarate to form succinyl-CoA, carbon
dioxide, and NADPH, EC No. 1.8.1.4; protein accession number
NP_414658).

| Protein Name | Protein Accession No. |
|---|---|
| Dihydrolipoamide dehydrogenase | NP_000099 |
| Dihydrolipoyl dehydrogenase | XP_003382649 |
| Dihydrolipoamide dehydrogenase | XP_002492166 |
| Lpd1p | NP_116635 |
| Dihydrolipoamide dehydrogenase | YP_726787 |
| Dihydrolipoamide dehydrogenase | NP_390286 |

TABLE 1B-7
Suitable homologues for the SucD protein (succinate-semialdehyde
dehydrogenase from Clostridium kluyveri, EC No. 1.2.1.76, which
converts succinyl-CoA to succinyl semialdehyde; protein sequence
in WO 2011/100601).

| | Protein |
|---|---|
| Protein Name | Accession No. |
| CoA-dependent succinate semialdehyde dehydrogenase | AAA92347 |
| Succinate-semialdehyde dehydrogenase [NAD(P)+] | ZP_06559980 |
| Succinate-semialdehyde dehydrogenase [NAD(P)+] | ZP_05401724 |
| Aldehyde-alcohol dehydrogenase family protein | ZP_07821123 |
| Succinate-semialdehyde dehydrogenase [NAD(P)+] | ZP_06983179 |
| Succinate-semialdehyde dehydrogenase | YP_001928839 |
| hypothetical protein CLOHYLEM_05349 | ZP_03778292 |
| Succinate-semialdehyde dehydrogenase [NAD(P)+] | YP_003994018 |
| Succinate-semialdehyde dehydrogenase | NP_904963 |

TABLE 1B-8
Suitable homologues for the Mcr protein (malonyl-CoA reductase from
Sulfolobus tokodaii, EC No. 1.2.1.75 (1.2.1.—), which acts on
malonyl-CoA (succinyl-CoA) to form malonyl semialdehyde (succinyl
semialdehyde); protein sequence in Gene/Protein ID 2).

| | Protein |
|---|---|
| Protein Name | Accession No. |
| short-chain alcohol dehydrogenase | YP_004863680 |
| short-chain dehydrogenase/reductase SDR | YP_001277512 |
| short-chain dehydrogenase/reductase SDR | YP_001433009 |
| short-chain dehydrogenase/reductase SDR | YP_001636209 |
| short-chain dehydrogenase/reductase SDR | YP_002462600 |
| short-chain dehydrogenase/reductase SDR | YP_002570540 |

TABLE 1B-9
Suitable homologues for the KgdM protein (alpha-ketoglutarate
decarboxylase, from Mycobacterium tuberculosis, EC No. 4.1.1.71,
which acts on alpha-ketoglutarate to produce succinate semialdehyde
and carbon dioxide; protein acc. no. NP_335730).

| Protein Name | Protein Accession No. |
|---|---|
| Alpha-ketoglutarate decarboxylase | YP_001282558 |
| Alpha-ketoglutarate decarboxylase | NP_854934 |
| 2-oxoglutarate dehydrogenase sucA | ZP_06454135 |
| 2-oxoglutarate dehydrogenase sucA | ZP_04980193 |
| Alpha-ketoglutarate decarboxylase | NP_961470 |
| Alpha-ketoglutarate decarboxylase | YP_001852457 |
| Alpha-ketoglutarate decarboxylase | NP_301802 |
| Alpha-ketoglutarate decarboxylase | ZP_05215780 |
| Alpha-ketoglutarate decarboxylase | YP_001702133 |

TABLE 1B-10
Suitable homologues for the KgdP protein (Alpha-ketoglutarate
decarboxylase, from Pseudonocardia dioxanivorans CB1190,
EC No. 4.1.1.n, which acts on alpha-ketoglutarate to produce
succinate semialdehyde and carbon dioxide; protein acc. no.
YP_004335105).

| Protein Name | Protein Accession No. |
|---|---|
| Alpha-ketoglutarate decarboxylase | ZP_08119245 |
| 2-oxoglutarate dehydrogenase, E1 component | ZP_09743222 |
| Alpha-ketoglutarate decarboxylase | YP_705947 |
| Alpha-ketoglutarate decarboxylase | NP_961470 |
| Alpha-ketoglutarate decarboxylase | ZP_08024348 |
| Alpha-ketoglutarate decarboxylase | YP_003343675 |
| kgd gene product | NP_737800 |
| 2-oxoglutarate dehydrogenase complex, | YP_004223349 |
| dehydrogenase (E1) component | |
| 2-oxoglutarate dehydrogenase (succinyl- | EJF35718 |
| transferring), E1 component | |

TABLE 1B-11
Suitable homologues for the KgdS protein (2-oxoglutarate
decarboxylase, from Synechococcus sp. PCC 7002, EC No.
4.1.1.n, which acts on alpha-ketoglutarate to produce succinate
semialdehyde and carbon dioxide; protein acc. no. ACB00744.1).

| Protein Name | Protein Accession No. |
|---|---|
| Alpha-ketoglutarate decarboxylase | YP_001282558 |
| Alpha-ketoglutarate decarboxylase | NP_854934 |
| 2-oxoglutarate dehydrogenase sucA | ZP_06454135 |
| 2-oxoglutarate dehydrogenase sucA | ZP_04980193 |
| Alpha-ketoglutarate decarboxylase | NP_961470 |
| Alpha-ketoglutarate decarboxylase | YP_001852457 |
| Alpha-ketoglutarate decarboxylase | NP_301802 |
| Alpha-ketoglutarate decarboxylase | ZP_05215780 |
| Alpha-ketoglutarate decarboxylase | YP_001702133 |

TABLE 1B-12
Suitable homologues for the YneI (Sad) protein (succinate
semialdehyde dehydrogenase, NAD+-dependent, from
Escherichia coli, EC No. 1.2.1.24, which acts on glutarate
semialdehyde (succinic semialdehyde) to produce glutarate
(succinate); Protein acc. no. NP_416042 (Fuhrer et
al., J Bacteriol. 2007 Nov; 189(22): 8073-8. Dennis and
Valentin, U.S. Pat. No. 6,117,658)).

| Protein Name | Protein Accession No. |
|---|---|
| Succinate semialdehyde dehydrogenase | NP_805238 |
| Putative aldehyde dehydrogenase | YP_002919404 |
| Aldehyde dehydrogenase | NP_745295 |
| Aldehyde dehydrogenase | ZP_03269266 |
| Aldehyde dehydrogenase | ZP_05726943 |
| Aldehyde dehydrogenase | YP_001906721 |
| Hypothetical protein | BAF01627 |
| Aldehyde dehydrogenase | ZP_03739186 |
| Succinate-semialdehyde dehydrogenase | NP_637690 |

TABLE 1B-13
Suitable homologues for the GabD protein (succinate semialdehyde
dehydrogenase, NADP+-dependent, from Escherichia coli,
EC No. 1.2.1.20, which acts on glutarate semialdehyde (or succinic
semialdehyde) to produce glutarate (or succinate); Protein acc. no.
NP_417147 (Riley et al., Nucleic Acids Res. 34 (1), 1-9 (2006))).

| | Protein |
|---|---|
| Protein Name | Accession No. |
| Succinate-semialdehyde dehydrogenase I | ZP_05433422 |
| Succinate-semialdehyde dehydrogenase (NAD(P)(+)) | YP_001744810 |
| hypothetical protein CIT292_04137 | ZP_03838093 |
| Succinate-semialdehyde dehydrogenase I | YP_002638371 |
| Succinate-semialdehyde dehydrogenase I | YP_001333939 |
| Succinate-semialdehyde dehydrogenase I | NP_742381 |
| Succinate-semialdehyde dehydrogenase (NAD(P)(+)) | YP_002932123 |
| Succinate-semialdehyde dehydrogenase I | YP_001951927 |
| Succinate-semialdehyde dehydrogenase I | YP_298405 |

TABLE 1B-14
Suitable homlogues for protein AstD (succinylglutamic semialdehyde
dehydrogenase from Escherichia coli, EC No. 1.2.1.—, which
acts upon succinylglutamic semialdehyde (succinate semialdehyde)
to produce succinylglutamate (succinate); protein accession no.
NP_416260).

| Protein Name | Protein Accession No. |
|---|---|
| Succinylglutamic semialdehyde dehydrogenase | YP_002382476 |
| Hypothetical protein D186_18882 | ZP_16280274 |
| Succinylglutamic semialdehyde dehydrogenase | YP_003942089 |
| Succinylglutamic semialdehyde dehydrogenase | ZP_16225314 |
| Succinylglutamic semialdehyde dehydrogenase | YP_005933902 |
| Succinylglutamic semialdehyde dehydrogenase | YP_005431041 |
| Succinylglutamic semialdehyde dehydrogenase | ZP_10352779 |
| Succinylglutamic semialdehyde dehydrogenase | ZP_10036944 |
| Succinylglutamic semialdehyde dehydrogenase | YP_004730031 |

TABLE 1B-15
Suitable homologues for the SsaR_{At}protein (succinic
semialdehyde reductase, from Arabidopsis thaliana, EC No.
1.1.1.61, which acts on succinate semialdehyde to produce
4-hydroxybutyrate; protein acc. no. AAK94781).

| Protein Name | Protein Accession No. |
|---|---|
| 6-phosphogluconate dehydrogenase NAD- | XP_002885728 |
| binding domain-containing protein | |
| Hypothetical protein isoform 1 | XP_002266252 |
| Predicted protein | XP_002320548 |
| Hypothetical protein isoform 2 | XP_002266296 |
| Unknown | ACU22717 |
| 3-hydroxyisobutyrate dehydrogenase, putative | XP_002524571 |
| Unknown | ABK22179 |
| Unknown | ACJ85049 |
| Predicted protein | XP_001784857 |

TABLE 1B-16
Suitable homologues for the YqhD protein (NADP-dependent
alkdehyde dehydrogenase, from Escherichia coli, EC. No.
1.1.1.61, which acts on succinate semialdehyde
to produce 4-hydroxybutyrate; protein acc. no. NP_417484).

| Protein Name | Protein Accession No. |
|---|---|
| Alcohol dehydrogenase | YP_002638761 |
| NADP-dependent alcohol dehydrogenase | YP_005625617 |
| Alcohol dehydrogenase yqhD | YP_005728679 |
| Conserved hypothetical protein | YP_003041737 |
| Alcohol dehydrogenase YqhD | YP_004953646 |
| Fe-dependent alcohol dehydrogenase | YP_007011870 |
| Putative Fe- and NAD(P)-dependent aldehyde | YP_005946648 |
| dehydrogenase acting against short chain | |
| aldehyde | |

TABLE 1B-17
Suitable homologues for the YihU protein (succinate
semialdehyde reductase, from Escherichia coli,
EC No. 1.1.1.61, which acts on succinate
semialdehyde to produce 4-hydroxybutyrate; protein
acc. no. NP_418318).

| Protein Name | Protein Accession No. |
|---|---|
| Oxidoreductase | NP_807241 |
| Putative oxidoreductase | YP_005240289 |
| Protein YihU | YP_004954328 |
| NADH-dependent gamma-hydroxybutyrate | YP_003212537 |
| dehydrogenase | |
| Oxidoreductase yihU | YP_006522377 |

TABLE 1B-18
Suitable homologues for the FucO_{I6L-L7V} protein
(L-1,2-propanediol oxidoreductase, from Escherichia coli,
EC No. 1.1.1.77, which acts on succinate semialdehyde
to produce 4-hydroxybutyrate).

| Protein Name | Protein Accession No. |
|---|---|
| L-1,2-propanediol oxidoreductase | YP_001459571 |
| Lactaldehyde reductase | ZP_12475782 |
| L-1,2-propanediol oxidoreductase | YP_001455658 |
| Lactaldehyde reductase | ZP_17109585 |
| L-1,2-propanediol oxidoreductase | YP_003294352 |
| L-1,2-propanediol oxidoreductase | YP_002988900 |
| L-1,2-propanediol oxidoreductase | ZP_09185179 |
| Lactaldehyde reductase | ZP_06759418 |
| Alcohol dehydrogenase | ZP_05943499 |

TABLE 1B-19
Suitable homologues for the OrfZ protein (CoA transferase,
from Clostridium kluyveri DSM 555, EC No. 2.8.3.n, which
acts on 4-hydroxybutyrate to produce 4-hydroxybutyryl CoA;
protein acc. no. AAA92344).

| Protein Name | Protein Accession No. |
|---|---|
| 4-Hydroxybutyrate coenzyme A transferase | YP_001396397 |
| Acetyl-CoA hydrolase/transferase | ZP_05395303 |
| Acetyl-CoA hydrolase/transferase | YP_001309226 |
| 4-Hydroxybutyrate coenzyme A transferase | NP_781174 |
| 4-Hydroxybutyrate coenzyme A transferase | ZP_05618453 |
| Acetyl-CoA hydrolase/transferase | ZP_05634318 |
| 4-Hydroxybutyrate coenzyme A transferase | ZP_00144049 |
| Hypothetical protein ANASTE_01215 | ZP_02862002 |
| 4-Hydroxybutyrate coenzyme A transferase | ZP_07455129 |
| 4-Hydroxybutyrate coenzyme A transferase | YP_005014371 |
| hypothetical protein FUAG_02467 | ZP_10973595 |
| Acetyl-CoA hydrolase/transferase | ZP_10325539 |
| 4-Hydroxybutyrate coenzyme A transferase | ZP_10895308 |
| 4-Hydroxybutyrate coenzyme A transferase | ZP_15973607 |
| Acetyl-CoA hydrolase/transferase | YP_003639307 |
| 4-Hydroxybutyrate coenzyme A transferase | ZP_08514074 |
| Succinyl:benzoate coenzyme A transferase | YP_006721017 |
| 4-Hydroxybutyrate coenzyme A transferase | YP_003961374 |

TABLE 1B-20
Suitable homologues for the Buk1 protein (butyrate kinase I,
from Clostridium acetobutylicum ATCC824, EC No. 2.7.2.7,
which acts on 4-hydroxybutyrate to produce 4-hydroxybutyryl
phosphate).

| Protein Name | Protein Accession No. |
|---|---|
| Butyrate kinase | YP_001788766 |
| Butyrate kinase | YP_697036 |
| Butyrate kinase | YP_003477715 |
| Butyrate kinase | YP_079736 |
| Acetate and butyrate kinase | ZP_01667571 |
| Butyrate kinase | YP_013985 |
| Butyrate kinase | ZP_04670620 |
| Butyrate kinase | ZP_04670188 |
| Butyrate kinase | ZP_07547119 |

TABLE 1B-21
Suitable homologues for the Buk2 protein (butyrate kinase II,
from Clostridium acetobutylicum ATCC824, EC No. 2.7.2.7,
which acts on 4-hydroxybutyrate to produce 4-hydroxybutyryl
phosphate).

| Protein Name | Protein Accession No. |
|---|---|
| Butyrate kinase | YP_001311072 |
| hypothetical protein CLOSPO_00144 | ZP_02993103 |
| hypothetical protein COPEUT_01429 | ZP_02206646 |
| butyrate kinase | EFR5649 |
| butyrate kinase | ZP_0720132 |
| butyrate kinase | YP_0029418 |
| butyrate kinase | YP_002132418 |
| butyrate kinase | ZP_05389806 |
| phosphate butyryltransferase | ADQ27386 |

TABLE 1B-22
Suitable homologues for the Ptb protein (phosphotransbutyrylase,
from Clostridium acetobutylicum ATCC824, EC No. 2.3.1.19,
which acts on 4-hydroxybutyryl phosphate to produce
4-hydroxybutyryl CoA).

| Protein Name | Protein Accession No. |
|---|---|
| Phosphate butyryltransferase | YP_001884531 |
| Hypothetical protein COPCOM_01477 | ZP_03799220 |
| Phosphate butyryltransferase | YP_00331697 |
| Phosphate butyryltransferase | YP_004204177 |
| Phosphate butyryltransferase | ZP_05265675 |
| Putative phosphate acetyl/butyryltransferase | ZP_05283680 |
| Bifunctional enoyl-CoA hydratase/phosphate | YP_426556 |
| acetyltransferase | |
| Hypothetical protein CLOBOL_07039 | ZP_02089466 |
| Phosphate butyryltransferase | YP_003564887 |

TABLE 1B-23
Suitable homologues for the Polyhydroxyalkanoate synthase
proteins (PhaC3/C1* fusion protein from Pseudomonas putida
and Ralstonia eutropha JMP134; PhaC183* fusion protein
from Ralstonia eutropha H16 and Ralstonia sp. S-6;
EC No. 2.3.1.n, which acts on (R)-3-hydroxybutyryl-CoA or
4-hydroxybutyryl-CoA + [(R)-3-hydroxybutanoate-co-4
hydroxybutanoate]n to produce [(R)-3-hydroxybutanoate-co-4-
hydroxybutanoate](n + 1) + CoA and also acts on
4-hydroxybutyryl-CoA + [4-hydroxybutanoate]n to
produce [4-hydroxybutanoate](n + 1) + CoA).

| Protein Name | Protein Accession No. |
|---|---|
| Poly(R)-hydroxyalkanoic acid synthase, class I | YP_295561 |
| Poly(3-hydroxybutyrate) polymerase | YP_583508 |
| intracellular polyhydroxyalkanoate synthase | ADL70203 |
| poly(R)-hydroxyalkanoic acid synthase, class I | ZP_04764634 |
| poly-beta-hydroxybutyrate polymerase | CAH35535 |
| poly-deta-hydroxybutyric acid synthase | AAD01209 |
| PHB polymerase | AAB06755 |
| Poly(3-hydroxyalkanoate) polymerase | ZP_00942942 |
| poly-beta-hydroxybutyrate polymerase | EFF76436 |
| poly-beta-hydroxybutyrate polymerase | ACR28619 |
| poly(3-hydroxyalkanoate) synthase | BAA17430 |
| poly-beta-hydroxybutyrate polymerase | YP_004360851 |
| phaC2 gene product | YP_583821 |
| polyhydroxyalkanoic acid synthase | EGF41868 |
| poly(R)-hydroxyalkanoic acid synthase, class I | ZP_10719804 |
| polyhydroxyalkanoic acid synthase | YP_003752369 |
| PHA synthase | CAA47035 |
| poly(R)-hydroxyalkanoic acid synthase, class I | ABM42250 |
| PhaC | AAF23364 |
| polyhydroxyalkanoic acid synthase | AAW65074 |
| intracellular polyhydroxyalkanoate synthase | ADM24646 |
| poly(R)-hydroxyalkanoic acid synthase | YP_283333 |
| polyhydroxybutyrate synthase | AAL17611 |
| polyhydroxyalkanoate synthase | AAD53179 |
| polyhydroxyalkanoate synthase | AAA72004 |
| Poly(R)-hydroxyalkanoic acid synthase, class I | ABF52226 |
| Poly-beta-hydroxybutyrate polymerase | ZP_02489627 |
| probable poly-beta-hydroxybutyrate polymerase | CAD15333 |
| transmembrane protein | |
| poly(R)-hydroxyalkanoic acid synthase, class I | ZP_08961344 |
| PHA synthase | BAA21815 |
| poly-beta-hydroxybutyrate polymerase | YP_003977718 |
| poly(3-hydroxybutyrate) polymerase PhaC | YP_004685292 |
| poly(R)-hydroxyalkanoic acid synthase | YP_983028 |
| poly(R)-hydroxyalkanoic acid synthase, class I | ABO54722 |
| PHA synthase | BAA33155 |
| poly(R)-hydroxyalkanoic acid synthase, class I | ZP_02382303 |
| poly(R)-hydroxyalkanoic acid synthase | YP_001003639 |
| polyhydroxyalkanoic acid synthase | ZP_10443466 |
| poly-beta-hydroxybutyrate polymerase protein | CAQ36337 |
| polyhydroxyalkanoate synthase | ABN71571 |
| PHB synthase I | BAA36200 |
| poly-deta-hydroxybutyric acid synthase | AAD01209.1 |
| PHA synthase | BAE20054 |
| Poly(3-hydroxybutyrate) polymerase | YP_725940 |
| polyhydroxyalkanoic acid synthase | YP_002005374 |

TABLE 1B-24
Suitable homologues for the SucC protein (beta-subunit
of succinyl-CoA synthetase from Escherichia coli, EC
No. 6.2.1.5, which reversibly converts succinyl-CoA to
succinate and ATP; protein accession no. NP_415256).

| Protein Name | Protein Accession No. |
|---|---|
| Succinyl-CoA synthetase, beta subunit | NP_455294 |
| Succinyl-CoA synthetase, beta subunit | YP_001007130 |
| Succinyl-CoA synthetase, beta subunit | YP_003209697 |
| Succinyl-CoA synthetase, beta subunit | YP_001669983 |
| Succinyl-CoA synthetase, beta subunit | NP_389491 |
| Succinyl-CoA synthetase, beta subunit | YP_725064 |

TABLE 1B-25
Suitable homologues for the SucD protein (alpha-subunit of succinyl-CoA
synthetase from Escherichia coli, EC No. 6.2.1.5, which reversibly
converts succinyl-CoA to succinate and ATP; protein accession
no. NP_415257).

| Protein Name | Protein Accession No. |
|---|---|
| Succinyl-CoA synthetase subunit alpha | NP_455295 |
| Succinyl-CoA synthetase subunit alpha | YP_001007129 |
| Succinyl-CoA synthetase subunit alpha | YP_003209698 |
| Succinyl-CoA synthetase subunit alpha | YP_001669982 |
| Succinyl-CoA synthetase subunit alpha | NP_389492 |
| Succinyl-CoA synthetase subunit alpha | YP_725065 |

TABLE 1B-26
Suitable homologues for the Frd_g protein (fumarate reductase from
Trpanosoma brucei, EC No. 1.3.1.6, which acts on fumarate to produce
succinate; protein accession no. XP_844767).

| Protein Name | Protein Accession No. |
|---|---|
| Fumarate reductase (NADH) | XP_567271 |
| Hypothetical protein AN5909.2 | XP_663513 |
| NADH-dependent fumarate reductase | XP_810232 |
| Putative NADH-dependent fumarate reductase | XP_001468932 |
| Putative NADH-dependent fumarate reductase | XP_001568220 |

TABLE 1B-27
Suitable homologues for the AceA protein (isocitrate lyase from
Escherichia coli, EC No. 4.1.3.1, which acts on isocitrate to produce
succinate and glyoxylate; protein accession no. NP_418439).

| Protein Name | Protein Accession No. |
|---|---|
| Isocitrate lyase | NP_188809 |
| Isocitrate lyase | XP_002490461 |
| Icl1p | NP_010987 |
| Isocitrate lyase | YP_001669914 |
| Isocitrate lyase | YP_726676 |
| Isocitrate lyase | YP_005151232 |
| Isocitrate lyase | YP_005641374 |

TABLE 1B-28
Suitable homologues for the AceB protein (malate synthase from
Escherichia coli, EC No. 2.3.3.9, which acts on gloxylate and
acetyl-CoA to produce malate; protein accession no. NP_418438).

| Protein Name | Protein Accession No. |
|---|---|
| Malate synthase | NP_001190219 |
| Malate synthase G | YP_001666631 |
| Malate synthase | XP_002490592 |
| Mls1p | NP_014282 |
| Malate synthase | YP_726682 |
| Malate synthase | YP_001059507 |
| malate synthase G | YP_005616458 |
| malate synthase A | YP_004922007 |
| Malate synthase | YP_004893031 |

TABLE 1B-29
Suitable homologues for Ndk (NADH kinase from Aspergillus nidulans,
EC No. 2.7.1.86, which acts upon NADH and ATP to produce NADPH;
protein accession no. XP_682106).

| Protein Name | Protein Accession No. |
|---|---|
| Poly(p)/ATP NAD kinase, putative | XP_002402575 |
| Predicted protein | XP_002298393 |
| NADH kinase, putative | XP_002532123 |
| NADH kinase, mitochondrial precursor, putative | XP_002419594 |
| Mitochondrial NADH kinase Pos5 (predicted) | NP_594371 |

### Suitable Extrachromosomal Vectors and Plasmids

A “vector,” as used herein, is an extrachromosomal replicon, such as a plasmid, phage, or cosmid, into which another DNA segment may be inserted so as to bring about the replication of the inserted segment. Vectors vary in copy number, depending on their origin of replication, and size. Vectors with different origins of replication can be propagated in the same microbial cell unless they are closely related such as pMB1 and ColE1. Suitable vectors to express recombinant proteins can constitute pUC vectors with a pMB1 origin of replication having 500-700 copies per cell, pBluescript vectors with a ColE1 origin of replication having 300-500 copies per cell, pBR322 and derivatives with a pMB1 origin of replication having 15-20 copies per cell, pACYC and derivatives with a p15A origin of replication having 10-12 copies per cell, and pSC101 and derivatives with a pSC101 origin of replication having about 5 copies per cell as described in the QIAGEN® Plasmid Purification Handbook (found on the world wide web at: //kirshner.med.harvard.edu/files/protocols/QIAGEN_QIAGENPlasmidPurification_EN.pdf). A widely used vector is pSE380 that allows recombinant gene expression from an IPTG-inducible trc promoter (Invitrogen, La Jolla, Calif.).

### Suitable Strategies and Expression Control Sequences for Recombinant Gene Expression

Strategies for achieving expression of recombinant genes in E. coli have been extensively described in the literature (Gross, Chimica Oggi 7(3):21-29 (1989); Olins and Lee, Cur. Op. Biotech. 4:520-525 (1993); Makrides, Microbiol. Rev. 60(3):512-538 (1996); Hannig and Makrides, Trends in Biotech. 16:54-60 (1998)). Expression control sequences can include constitutive and inducible promoters, transcription enhancers, transcription terminators, and the like which are well known in the art. Suitable promoters include, but are not limited to, P_{lac}, P_{tac}, P_{trc}, P_{R}, P_{L}, Pₜᵣₚ, P_{phoA}, Pₐᵣₐ, P_{uspA}, P_{rpsU}, P_{syn} (Rosenberg and Court, Ann. Rev. Genet. 13:319-353 (1979); Hawley and McClure, Nucl. Acids Res. 11 (8):2237-2255 (1983); Harley and Raynolds, Nucl. Acids Res. 15:2343-2361 (1987); also at the world wide web at ecocyc.org and partsregistry.org).

Exemplary promoters are:

(SEQ ID NO: 1)
P_{syn1}(a.k.a. P_{synA}) (5′-TTGACAGCTAGCTCAGTCCTAGGTATAATGCTAGC-3′),
(SEQ ID NO: 2)
P_{synC} (5′-TTGACAGCTAGCTCAGTCCTAGGTACTGTGCTAGC-3′),
(SEQ ID NO: 3)
P_{synE} (5′-TTTACAGCTAGCTCAGTCCTAGGTATTATGCTAGC-3′),
(SEQ ID NO: 4)
P_{synH} (5′-CTGACAGCTAGCTCAGTCCTAGGTATAATGCTAGC-3′),
(SEQ ID NO: 5)
P_{synK} (5′-TTTACGGCTAGCTCAGTCCTAGGTACAATGCTAGC-3′),
(SEQ ID NO: 6)
P_{synM} (5′-TTGACAGCTAGCTCAGTCCTAGGGACTATGCTAGC-3′),
(SEQ ID NO: 7)
P_{trc} (5′-TTGACAATTAATCATCCGGCTCGTATAATG-3′),
(SEQ ID NO: 8)
P_{tac} (5′-TTGACAATTAATCATCGTCGTATAATGTGTGGA-3′),
(SEQ ID NO: 9)
Pₜₑₜ (5′-TCCCTATCAGTGATAGAGATTGACATCCCTATCAGTGATAGAGATACTGAGCAC-3′),
(SEQ ID NO: 10)
Pₓ (5′-TCGCCAGTCTGGCCTGAACATGATATAAAAT-3′),
(SEQ ID NO: 11)
P_{uspA} (5′-AACCACTATCAATATATTCATGTCGAAAATTTGTTTATCTAACGAGTAAGCAAGGCGGATTG
ACGGATCATCCGGGTCGCTATAAGGTAAGGATGGTCTTAACACTGAATCCTTACGGCTGGGTAAGCCCCGC
GCACGTAGTTCGCAGGACGCGGGTGACGTAACGGCACAAGAAACG-3′),
(SEQ ID NO: 12)
P_{rpsU} (5′-ATGCGGGTTGATGTAAAACTTTGTTCGCCCCTGGAGAAAGCCTCGTGTATACTCCTCACCC
TTATAAAAGTCCCTTTCAAAAAAGGCCGCGGTGCTTTACAAAGCAGCAGCAATTGCAGTAAAATTCCGCAC
CATTTTGAAATAAGCTGGCGTTGATGCCAGCGGCAAAC-3′).
(SEQ ID NO: 13)
P_{synAF7} (5′-TTGACAGCTAGCTCAGTCCTAGGTACAGTGCTAGC-3′)
(SEQ ID NO: 14)
P_{synAF3} (5′-TTGACAGCTAGCTCAGTCCTAGGTACAATGCTAGC-3′)

Exemplary terminators are:

(SEQ ID NO: 15)
TₜᵣₚL (5-CTAATGAGCGGGCTTTTTTTTGAACAAAA-3′),
(SEQ ID NO: 16)
T₁₀₀₆ (5-AAAAAAAAAAAACCCCGCTTCGGCGGGGTTTTTTTTTT-3′),
(SEQ ID NO: 17)
T_{rrnB1} (5-ATAAAACGAAAGGCTCAGTCGAAAGACTGGGCCTTTCGTTTT
AT-3′),
(SEQ ID NO: 18)
T_{rrnB2} (5-AGAAGGCCATCCTGACGGATGGCCTTTT-3′).

### Construction of Recombinant Hosts

Recombinant hosts containing the necessary genes that will encode the enzymatic pathway for the conversion of a carbon substrate to P4HB may be constructed using techniques well known in the art.

Methods of obtaining desired genes from a source organism (host) are common and well known in the art of molecular biology. Such methods are described in, for example, Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, Md. (1999). For example, if the sequence of the gene is known, the DNA may be amplified from genomic DNA using polymerase chain reaction (Mullis, U.S. Pat. No. 4,683,202) with primers specific to the gene of interest to obtain amounts of DNA suitable for ligation into appropriate vectors. Alternatively, the gene of interest may be chemically synthesized de novo in order to take into consideration the codon bias of the host organism to enhance heterologous protein expression. Expression control sequences such as promoters and transcription terminators can be attached to a gene of interest via polymerase chain reaction using engineered primers containing such sequences. Another way is to introduce the isolated gene into a vector already containing the necessary control sequences in the proper order by restriction endonuclease digestion and ligation. One example of this latter approach is the BioBrick™ technology (www.biobricks.org) where multiple pieces of DNA can be sequentially assembled together in a standardized way by using the same two restriction sites.

In addition to using vectors, genes that are necessary for the enzymatic conversion of a carbon substrate to P4HB can be introduced into a host organism by integration into the chromosome using either a targeted or random approach. For targeted integration into a specific site on the chromosome, the method generally known as Red/ET recombineering is used as originally described by Datsenko and Wanner (Proc. Natl. Acad. Sci. USA, 2000, 97, 6640-6645). Random integration into the chromosome involves using a mini-Tn5 transposon-mediated approach as described by Huisman et al. (U.S. Pat. Nos. 6,316,262 and 6,593,116).

### Culturing of Host to Produce P4HB Biomass

In general, the recombinant host is cultured in a medium with a carbon source and other essential nutrients to produce the P4HB biomass by fermentation techniques either in batches or continuously using methods known in the art. Additional additives can also be included, for example, antifoaming agents and the like for achieving desired growth conditions. Fermentation is particularly useful for large scale production. An exemplary method uses bioreactors for culturing and processing the fermentation broth to the desired product. Other techniques such as separation techniques can be combined with fermentation for large scale and/or continuous production.

As used herein, the term “feedstock” refers to a substance used as a carbon raw material in an industrial process. When used in reference to a culture of organisms such as microbial or algae organisms such as a fermentation process with cells, the term refers to the raw material used to supply a carbon or other energy source for the cells. Carbon sources useful for the production of P4HB include simple, inexpensive sources, for example, glucose, levoglucosan, sucrose, lactose, fructose, xylose, maltose, arabinose and the like alone or in combination. In other embodiments, the feedstock is molasses or starch, fatty acids, vegetable oils or a lignocellulosic material and the like. It is also possible to use organisms to produce the P4HB biomass that utilizes synthesis gas (CO₂, CO and hydrogen) produced from renewable biomass resources and/or methane originating from landfill gas that can be used directly as feed stock or is converted to methanol.

Introduction of P4HB pathway genes allows for flexibility in utilizing readily available and inexpensive feedstocks. A “renewable” feedstock refers to a renewable energy source such as material derived from living organisms or their metabolic byproducts including material derived from biomass, often consisting of underutilized components like chaff or stover. Agricultural products specifically grown for use as renewable feedstocks include, for example, corn, soybeans, switchgrass and trees such as poplar, wheat, flaxseed and rapeseed, sugar cane and palm oil. As renewable sources of energy and raw materials, agricultural feedstocks based on crops are the ultimate replacement for declining oil reserves. Plants use solar energy and carbon dioxide fixation to make thousands of complex and functional biochemicals beyond the current capability of modern synthetic chemistry. These include fine and bulk chemicals, pharmaceuticals, nutraceuticals, flavanoids, vitamins, perfumes, polymers, resins, oils, food additives, bio-colorants, adhesives, solvents, and lubricants.

### Example 1

### Improved P4HB Production by Use of an α-Ketoglutarate Decarboxylase from Pseudonocardia dioxanivorans

Several metabolic pathways were proposed to generate succinic semialdehyde (SSA) from the tricarboxylic acid (TCA) cycle (reviewed by Steinbuchel and Lütke-Eversloh, Biochem. Engineering J. 16:81-96 (2003) and Efe et al., Biotechnology and Bioengineering 99:1392-1406 (2008)). One such pathway converts alpha-ketoglutarate to SSA via an alpha-ketoglutarate decarboxylase that is encoded by kgdM (Tian et al., Proc. Natl. Acad. Sci. U.S.A. 102:10670-10675 (2005)). Previous attempts to utilize the kgdM gene from Mycobacterium tuberculosis (Tian et al. Proc. Natl. Acad. Sci. U.S.A. 102:10670-10675 (2005); FIG. 1, Reaction number 6) for production of P4HB were not successful resulting in only very small amounts of P4HB (Van Walsem et al., Patent Application No. WO 2011100601 A1).

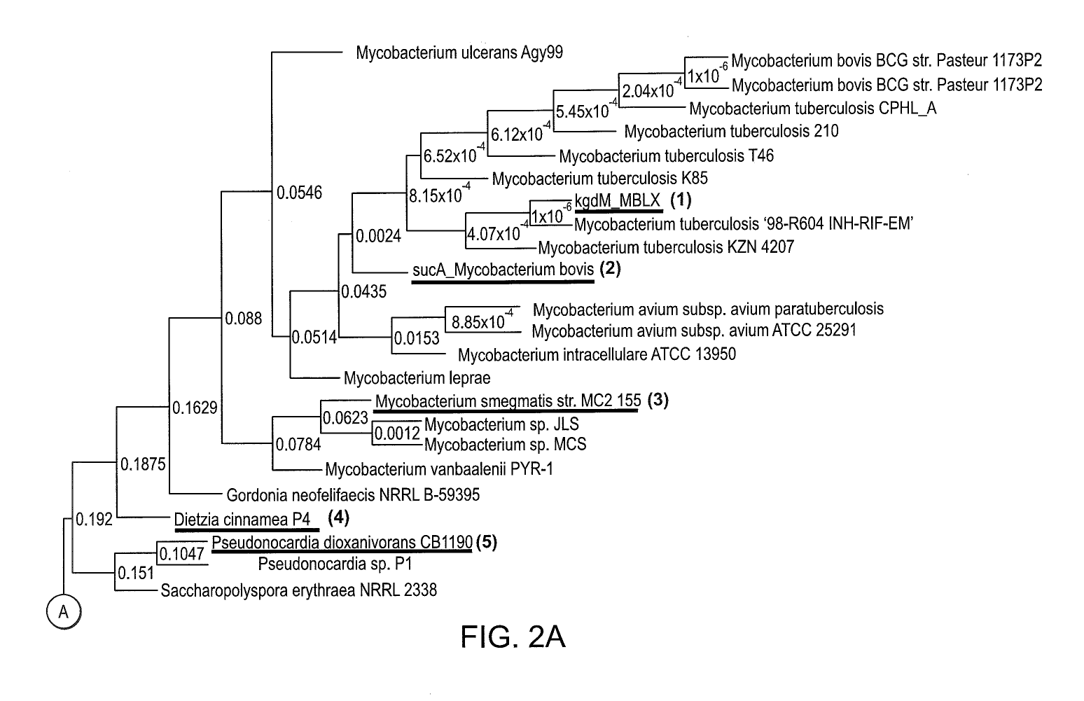

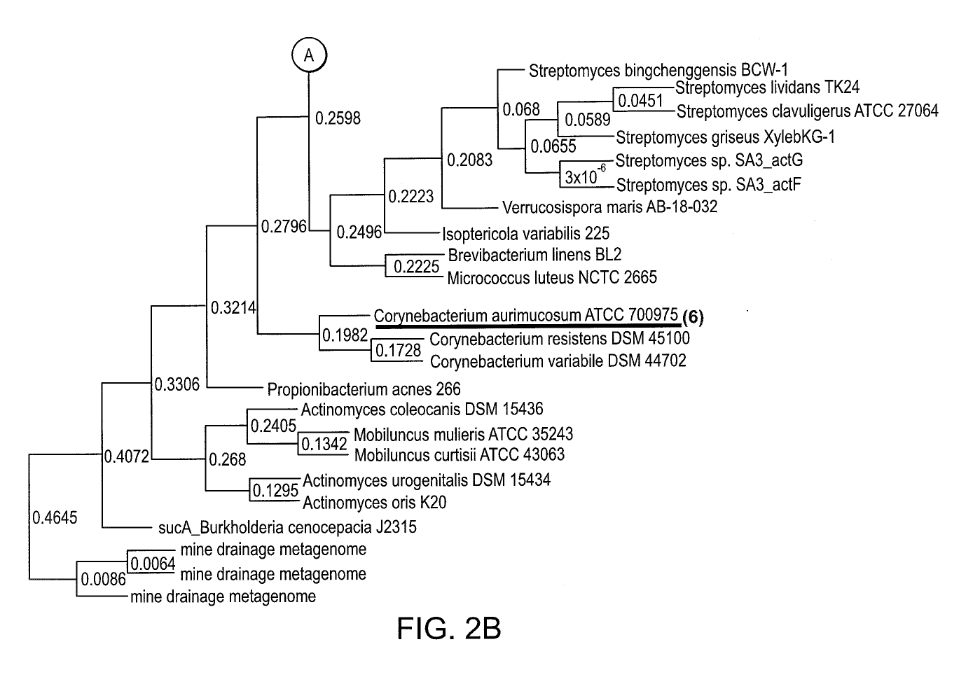

This example demonstrates that a homologue of the M. tuberculosis KgdM unexpectedly was able to produce significant amounts of P4HB when overproduced in recombinant host strains. BLASTP searches (Altschul, J. Mol. Biol. 219:555-65 (1991)) using the protein sequence of KgdM as query against the non-redundant protein database identified several homologues, which were aligned in a multiple sequence alignment using the MAFFT alignment algorithm available from the Geneious software package (Drummond, A. J. et al., Geneious v5.4 (2011); available on the world wide web at geneious.com). This alignment served as the input file to generate a phylogenetic tree using the Geneious Tree Builder with the Jukes-Cantor genetic distance model and the UPGMA Tree Build Model as shown in FIG. 2. Based on this phylogenetic tree, several close and more distant homologues were selected as gene targets. These included Mycobacterium bovis (Accession No. CAL71295), M. smegmatis (Accession No. A0R2B1), Dietzia cinnamea (Accession No. EFV91102), Corynebacterium aurimucosum (Accession No. ZP_{—}06042096), and Pseudonocardia dioxanivorans (Accession No. AEA27252; see FIG. 2). Using polymerase chain reaction (PCR), the native genes were amplified from genomic DNA of the native microbes of M. smegmatis, D. cinnamea, C. aurimucosum, and P. dioxanivorans using the well-known molecular biological techniques described above and were cloned into a plasmid downstream of a P_{trc} promoter. The kgdM* genes of M. tuberculosis and M. bovis were codon-optimized by DNA2.0 for optimal expression in E. coli host strains and were also cloned into the same plasmid downstream of a P_{trc} promoter.

Thus, the following six strains were constructed using the well-known biotechnology tools and methods described above, all of which contained chromosomal deletions of yneI and gabD as well as pykF and pykA and overexpressed the orfZ_{Ck} gene from Clostridium kluyveri, the E. coli ppc gene, the PHA synthase phaC3/C1*, and the ssaR_{At}* gene from Arabidopsis thaliana. All those genes are described in Table1A. Strain 1 served as a positive control expressing the sucD_{Ck}* gene from C. kluyveri that was previously shown to produce significant amounts of P4HB (Van Walsem et al., Patent Application No. WO 2011100601 A1). Strain 2 served as a negative control expressing the M. tuberculosis kgdM gene from the IPTG-inducible P_{trc} promoter. Strains 3 to 6 expressed the M. bovis, C. aurimucosum, P. dioxanivorans, and M. smegmatis kgd homologues, respectively, from the IPTG-inducible P_{trc} promoter (see Table 2).

TABLE 2
Microbial Strains used in Example 1

| | Relevant host genome | |
|---|---|---|
| Strains | deletions | Genes overexpressed |
| 1 | ΔyneI, ΔgabD, ΔpykF, ΔpykA | P_{uspA}-phaC3/C1*-ssaR_{At}*, P_{rpsU}-orfZ_{Ck}, P_{syn1}-ppc, |
| | | Pₜₑₜ-sucD_{Ck}* (Clostridium kluyveri) |
| 2 | ΔyneI, ΔgabD, ΔpykF, ΔpykA | P_{uspA}-phaC3/C1*-ssaR_{At}*, P_{rpsU}-orfZ_{Ck}, P_{syn1}-ppc, |
| | | P_{trc}-kgdM* (Mycobacterium tuberculosis) |
| 3 | ΔyneI, ΔgabD, ΔpykF, ΔpykA | P_{uspA}-phaC3/C1*-ssaR_{At}*, P_{rpsU}-orfZ_{Ck}, P_{syn1}-ppc, |
| | | P_{trc}-kgdM* (Mycobacterium bovis) |
| 4 | ΔyneI, ΔgabD, ΔpykF, ΔpykA | P_{uspA}-phaC3/C1*-ssaR_{At}*, P_{rpsU}-orfZ_{Ck}, P_{syn1}-ppc, |
| | | P_{trc}-kgd (Corynebacterium aurimucosum) |
| 5 | ΔyneI, ΔgabD, ΔpykF, ΔpykA | P_{uspA}-phaC3/C1*-ssaR_{At}*, P_{rpsU}-orfZ_{Ck}, P_{syn1}-ppc, |
| | | P_{trc}-kgd (Pseudonocardia dioxanivorans) |
| 6 | ΔyneI, ΔgabD, ΔpykF, ΔpykA | P_{uspA}-phaC3/C1*-ssaR_{At}*, P_{rpsU}-orfZ_{Ck}, P_{syn1}-ppc, |
| | | P_{trc}-kgd (Mycobacterium smegmatis) |

The strains were grown in a shake plate assay to examine production of P4HB. Three replicates of each of the six strains were cultured overnight in a sterile tube containing 3 mL of LB, 50 μg/mL kanamycin, and either 25 μg/mL chloramphenicol (for strain 1) or 100 mg/mL ampicillin (for strains 2-6). From this, 50 μL was added in triplicate to Duetz deep-well plate wells containing 450 μL of production medium and antibiotics as indicated above. The production medium consisted of 1× E2 minimal salts solution containing 15 g/L glucose, 2 mM MgSO₄, 1× Trace Salts Solution, and 100 μM IPTG to induce recombinant gene expression. 50×E2 stock solution consists of 1.275 M NaNH₄HPO₄.4H₂O, 1.643 M K₂HPO₄, and 1.36 M KH₂PO₄. 1000× stock Trace Salts Solution is prepared by adding per 1 L of 1.5 N HCL: 50 g FeSO₄.7H₂0, 11 g ZnSO₄.7H₂O, 2.5 g MnSO₄.4H₂O, 5 g CuSO₄.5H₂O, 0.5 g (NH₄)₆Mo₇O₂₄.4H₂O, 0.1 g Na₂B₄O₇, and 10 g CaCl₂.2H₂O. The shake plate was grown for 5 hours at 37° C. with shaking and then incubated at 30° C. for a total of 48 hours. Thereafter, production well sets were combined (1.5 mL total) and analyzed for polymer content. At the end of the experiment, cultures were spun down at 4150 rpm, washed once with distilled water, frozen at −80° C. for at least 30 minutes, and lyophilized overnight. The next day, a measured amount of lyophilized cell pellet was added to a glass tube, followed by 3 mL of butanolysis reagent that consists of an equal volume mixture of 99.9% n-butanol and 4.0 N HCl in dioxane with 2 mg/mL diphenylmethane as internal standard. After capping the tubes, they were vortexed briefly and placed on a heat block set to 93° C. for six hours with periodic vortexing. Afterwards, the tube was cooled down to room temperature before adding 3 mL distilled water. The tube was vortexed for approximately 10 s before spinning down at 620 rpm (Sorvall Legend RT benchtop centrifuge) for 2 min. 1 mL of the organic phase was pipetted into a GC vial, which was then analyzed by gas chromatography-flame ionization detection (GC-FID) (Hewlett-Packard 5890 Series II). The quantity of PHA in the cell pellet was determined by comparing against a standard curve for 4HB (for P4HB analysis). The 4HB standard curve was generated by adding different amounts of a 10% solution of γ-butyrolactone (GBL) in butanol to separate butanolysis reactions.

The results in Table 3 surprisingly show that only strain 5 expressing the kgd homologue from P. dioxanivorans produced P4HB at significant levels.

TABLE 3
Biomass and P4HB titer

| Strains | Biomass Titer (g/L) | P4HB Titer (% dcw) |
|---|---|---|
| 1 | 3.69 ± 0.07 | 18.0 ± 0.2% |
| 2 | 3.18 ± 0.12 | 3.0 ± 0.3% |
| 3 | 3.20 ± 0.01 | 3.0 ± 0.1% |
| 4 | 3.33 ± 0.16 | 2.0 ± 0.0% |
| 5 | 3.43 ± 0.05 | 12.0 ± 0.3% |
| 6 | 3.36 ± 0.08 | 3.0 ± 0.8% |

### Example 2

### Development of a Growth Selection Strategy to Obtain Genes with Improved α-Ketoglutarate Decarboxylase Activities

The P4HB titer of a recombinant host expressing the kgd homologue from P. dioxanivorans, hereafter called kgdP, was only about two thirds of the titer obtained in strains expressing the sucD_{Ck}* gene from Clostridium kluyveri (see Tables 2 and 3). Therefore, a growth selection method was developed to obtain mutated kgdP genes with improved α-ketoglutarate decarboxylase activity. For this, an E. coli MG1655 ΔsucAB strain was constructed that lacked the alpha-ketoglutarate dehydrogenase activity (FIG. 1, reaction 4). MG1655 containing the sucAB deletion was constructed using the well-known biotechnology tools and methods described above. This strain was unable to grow in E2 minimal medium supplemented with 2.0 g/L alpha-ketoglutarate as sole carbon source due to lack of alpha-ketoglutarate dehydrogenase (ΔsucAB) and any native alpha-ketoglutarate decarboxylase activity in E. coli cells. However, assuming a recombinant kgd gene was expressed in a ΔsucAB E. coli host that exhibited adequate levels of alpha-ketoglutarate decarboxylase activity, cells should be able to grow with alpha-ketoglutarate as sole carbon source by using the metabolic pathway reaction 6 (αKG→SSA) and reaction 7 (SSA→SUC) as shown in FIG. 1 to complete the interrupted TCA cycle. To test this assumption, the native kgdP gene was cloned into an expression vector and was shown to be unable to grow in E2 minimal media supplemented with 2.0 g/L alpha-ketoglutarate (data not shown). Therefore, hydroxylamine-induced random mutagenesis was performed as described in Sugimoto et al. (U.S. Pat. No. 5,919,694) to select for mutated kgdP genes that enable growth in E2 minimal medium supplemented with alpha-ketoglutarate as sole carbon source.

The wild-type kgdP gene was first cloned under the control of the Pₜ, promoter in pSE380, followed by hydroxylamine mutagenesis at 75° C. for 2 h. The mutagenesis solution was then transformed into an E. coli MG1655 ΔsucAB strain and plated on LB agar plates supplemented with appropriate antibiotics (100 μg/mL ampicillin and 25 μg/mL chloramphenicol) and incubated at 37° C. overnight. The next day, about one million individual colonies from multiple transformations were collected and pooled using 3 ml of 1× E2 buffer. 10 μl of the pooled mutant clones were subcultured into a shake flask containing 50 ml of growth selection medium consisting of 1× E2 minimal salts solution, 2 mM MgSO₄, 1× Trace Salts Solution, 10 μM IPTG, 100 μg/mL ampicillin, 25 μg/mL chloramphenicol, and 2 g/L alpha-ketoglutarate as sole carbon source. The 50×E2 stock solution and 1000× trace salts stock solution were prepared as described in Example 1. The shake flask culture was incubated at 30° C. with shaking at 250 rpm. The cell growth (OD_{600 nm}) was monitored periodically. After 2 days, the culture was able to grow to stationary phase resulting in an OD_{600 nm} of about 2.0. The plasmids were isolated from this shake flask culture using QIAprep Spin Miniprep Kit (Valencia, Calif.). The plasmid mixture was then transformed into an E. coli strain that contained chromosomal deletions in yneI, gabD, pykF, and pykA and overexpressed the orfZ_{Ck} gene from Clostridium kluyveri, the E. coli ppc gene, the PHA synthase phaC3/C1* gene, and the ssaR_{At}* gene from Arabidopsis thaliana. The transformation mix was then plated on 1× E2 minimal medium agar plates supplemented with 2 mM MgSO₄, 1× Trace Salts Solution, 10 g/L glucose as sole carbon source, 100 μg/mL ampicillin, 50 μg/mL kanamycin, and 100 μM IPTG. Finally, a very white colony indicating high P4HB production was selected. The plasmid of this exemplary clone was isolated and its DNA sequence of kgdP was established. The mutated kgdP, hereafter called kgdP-M38, contained three mutations within the coding sequence (Table 4). Two mutations at positions 696 and 3303 did not result in an amino acid change but impacted the codon frequency, whereas the mutation at position 2659 resulted in an alanine (Ala, A) change to threonine (Thr, T), which also impacted the codon frequency.

TABLE 4
Base pair changes in the kgdP-M38 coding sequence (CDS)

| Base pair (CDS) | Codon | Codon frequency | Amino acid |
|---|---|---|---|
| 696 | AAG → AAA | 24% → 76% | no change |
| 2659 | GCC → ACC | 25% → 43% | Ala887Thr |
| 3303 | GTG → GTA | 34% → 17% | no change |

### Example 3

### Improved P4HB Production by Expression of the Mutated α-Ketoglutarate Decarboxylase kgdP-M38 from Pseudonocardia dioxanivorans

Improved P4HB Production in Strains Expressing kgdP-M38

In this example P4HB production is compared in strains expressing the native kgdP versus the mutated kgdP-M38 from Pseudonocardia dioxanivorans. The following two strains were thus constructed using the well-known biotechnology tools and methods described above, both containing chromosomal deletions in yneI, gabD, pykF and pykA and overexpressing the orfZ_{Ck} gene from Clostridium kluyveri, the E. coli ppc gene, the PHA synthase phaC3/C1*, and the ssaR_{At}* gene from Arabidopsis thaliana. In addition, strain 7 expressed the native kgdP gene from the P_{trc} promoter, whereas strain 8 expressed the mutated kgdP-M38 also from the P_{trc} promoter (Table 5).

TABLE 5
Microbial Strains used in this section of Example 3

| Strains | Relevant host genome deletions | Genes overexpressed |
|---|---|---|
| 7 | ΔyneI, ΔgabD, ΔpykF, ΔpykA | P_{uspA}-phaC3/C1*-ssaR_{At}*, |
| | | P_{syn1}-ppc, P_{rpsU}- |
| | | orfZ_{Ck}, P_{trc}-kgdP |
| 8 | ΔyneI, ΔgabD, ΔpykF, ΔpykA | P_{uspA}-phaC3/C1*-ssaR_{At}*, |
| | | P_{syn1}-ppc, P_{rpsU}- |
| | | orfZ_{Ck}, P_{trc}-kgdP-M38 |

LB overnights of strains 7 and 8 were grown in 3 mL LB containing 50 μg/mL Km and 100 μg/mL Ap at 37° C. On the next day, the strains were grown in a shake plate at 37° C. for 5 hr which was followed by incubation of the shake plate at 30° C. for 39 hr using the same medium as described in Example 1 except that 30 g/L glucose was provided as the carbon source. Preparation and analysis of the cultures were carried out as described in Example 1.

As shown in Table 6, the P4HB titer of strain 8 expressing the mutated kgdP-M38 far exceeded the P4HB titer of strain 7 expressing the native kgdP gene.

TABLE 6
Biomass and P4HB titer

| Strains | Biomass Titer (g/L) | P4HB Titer (% dcw) |
|---|---|---|
| 7 | 3.42 ± 0.01 | 7.95 ± 2.40 |
| 8 | 4.60 ± 0.09 | 29.94 ± 0.58 |

Improved P4HB Production in Strains Expressing kgdP-M38 Together with sucDz_{Ck}*

In order to determine if expression of the mutated kgdP-M38 could increase P4HB titers in strains also expressing the sucD_{Ck}* gene from C. kluyveri, two strains were constructed that contained chromosomal deletions in yneI, gabD, pykF, and pykA and overexpressed the orfZ_{Ck} gene from C. kluyveri, the E. coli ppc gene, the PHA synthase phaC3/C1* gene, the ssaR_{At}* gene from A. thaliana and the sucD_{Ck}* gene from C. kluyveri. Strain 9 expressed the native kgdP gene from the P_{trc} promoter, whereas strain 10 expressed the mutated kgdP-M38 also from the P_{trc} promoter (Table 7).

TABLE 7
Microbial Strains used in this section of Example 3

| | Relevant host genome | |
|---|---|---|
| Strains | deletions | Genes overexpressed |
| 9 | ΔyneI, ΔgabD, ΔpykF, ΔpykA | Pₓ-phaC3/C1*, P_{uspA}- |
| | | sucD_{Ck}*-ssaR_{At}*, P_{syn1}- |
| | | ppc, P_{rpsU}-orfZ_{Ck}, P_{trc-}kgdP |
| 10 | ΔyneI, ΔgabD, ΔpykF, ΔpykA | Pₓ-phaC3/C1*, P_{uspA}- |
| | | sucD_{Ck}*-ssaR_{At}*, P_{syn1}- |
| | | ppc, P_{rpsU}-orfZ_{Ck}, P_{trc-}kgdP-M38 |

LB overnights of strains 9 and 10 were grown in 3 mL LB containing 25 μg/mL Cm and 100 μg/mL Ap at 37° C. On the next day, the strains were innoculated into a shake plate and incubated at 28° C. for 42 hr using the same medium as described in Example 1 except that 56.6 g/L glucose was provided as the carbon source. Parallel cultures of strains 9 and 10 were grown where IPTG was added to 0 or 100 μM to induce gene expression. Preparation and analysis of the cultures were carried out as described in Example 1.

As shown in Table 8, the P4HB titer produced by strain 9 expressing the native kgdP gene with 100 μM IPTG was not different from the non-induced, 0 μM IPTG control of the same strain. However, strain 10 expressing the mutated kgdP-M38 with 100 μM IPTG was significantly increased over the non-induced control strain 10, as well as the non-induced or induced strain 9 cells. This demonstrates that the combined expression of sucD_{Ck}* and mutated kgdP-M38 results in superior P4HB production.

TABLE 8
Biomass and P4HB titer

| Strains | [IPTG] (μM) | Biomass Titer (g/L) | P4HB Titer (% dcw) |
|---|---|---|---|
| 9 | 0 | 6.20 ± 0.12 | 47.57 ± 0.95 |
| | 100 | 6.22 ± 0.16 | 48.60 ± 2.19 |
| 10 | 0 | 6.31 ± 0.13 | 49.63 ± 0.46 |
| | 100 | 8.49 ± 0.01 | 60.72 ± 1.59 |

### Example 4

### Wild-Type Enzyme Activity of a Cyanobacterial α-Ketoglutarate Decarboxylase is Sufficient for Growth Recovery in Engineered E. coli Screening Strains

In a recent discovery, Zhang and Bryant (Science 334:1551-1553 (2011)) identified a 2-oxoglutaratedecarboxylase enzyme from Synechococcus sp. PCC 7002 that catalyzed the same metabolic reaction as KgdM or KgdP (FIG. 1, reaction 6). However, the amino acid sequence of the newly elucidated 2-oxoglutaratedecarboxylase was found to be different from the Kgd enzymes of M. tuberculosis and its homologues.

This example demonstrates that expression of the 2-oxoglutaratedecarboxylase gene, hereafter called kgdS, enables growth in the E. coli MG1655 ΔsucAB strain described in Example 2 when grown in E2 minimal medium supplemented with alpha-ketoglutarate as sole carbon source. The following three strains were constructed. Strain 11 was MG1655 that only harbored the empty vector and thus did not overexpress any recombinant gene. Strain 12 was the MG1655 host that contained a chromosomal deletion of sucAB and only harbored the empty vector. Strain 13 contained the same chromosomal deletion as strain 12, but expressed the kgdS gene from Synechococcus sp. PCC 7002 from a P_{trc} promoter (Table 9).

TABLE 9
Strains used in Example 4

| | Relevant host genome | |
|---|---|---|
| Strains | deletions | Genes overexpressed |
| 11 | Wild type (SucAB⁺) | None |
| 12 | ΔsucAB | None |
| 13 | ΔsucAB | P_{trc}-kgdS |
| | | (Synechococcus sp. PCC 7002) |

Strains 11, 12, and 13 were grown in liquid medium consisting of 1×E2 salts, 2 mM MgSO4, 1× Trace Salts Solution, 2 g/L α-ketoglutarate, 100 μg/mL ampicillin and 10 μM IPTG at 37° C. The composition of the 50×E2 salts stock solution and the 1000× Trace Salts Solution are given in Example 1. OD600 measurements were taken periodically in order to determine the growth rate.

As shown in Table 10, the positive control strain 11 exhibited a specific growth rate of 0.37 h⁻¹, whereas strain 12 containing the chromosomal deletion in sucAB, as expected, did not grow at all. Surprisingly, expression of kgdS from Synechococcus sp. PCC 7002 resulted in a fully restored specific growth rate of 0.36 in a sucAB deletion background strain.

TABLE 10
Growth rates with α-ketoglutarate as sole carbon source

| Strains | Specific Growth Rate (h⁻¹) |
|---|---|
| 11 | 0.37 |
| 12 | 0.00 |
| 13 | 0.36 |

### Example 5

### Improved P4HB Production by Expression of a 2-Oxoglutarate Decarboxylase from Synechococcus sp. PCC 7002

In this example P4HB production is compared in strains expressing either the sucD_{Ck}* from C. kluyveri or the mutated kgdP-M38 from P. dioxanivorans versus the kgdS from Synechococcus sp. PCC 7002. For this, three strains were constructed all containing chromosomal deletions in yneI, gabD, pykF, and pykA and overexpressing the orfZₐ gene from C. kluyveri, the E. coli ppc gene, the PHA synthase phaC3/C1* gene, and the ssaR_{At}* gene from A. thaliana. Strain 14 expressed the sucD_{Ck}* gene from C. kluyveri from a Pₜₑₜ promoter whereas strains 15 and 16 used a P_{trc} promoter to express the mutated kgdP-M38 from P. dioxanivorans and the native kgdS from Synechococcus sp. PCC 7002, respectively (Table 11).

TABLE 11
Microbial Strains used in Example 5

| | Relevant host genome | |
|---|---|---|
| Strains | deletions | Genes overexpressed |
| 14 | ΔyneI, ΔgabD, ΔpykF, ΔpykA | P_{uspA}-phaC3/C1*-ssaR_{At}*, |
| | | P_{syn1}-ppc, P_{rpsU}-orfZ_{Ck}, |
| | | Pₜₑₜ-sucD_{Ck}* |
| 15 | ΔyneI, ΔgabD, ΔpykF, ΔpykA | P_{uspA}-phaC3/C1*-ssaR_{At}*, |
| | | P_{syn1}-ppc, P_{rpsU}-orfZ_{Ck}, |
| | | P_{trc}-kgdP-M38 |
| | | (P. dioxanivorans) |
| 16 | ΔyneI, ΔgabD, ΔpykF, ΔpykA | P_{uspA}-phaC3/C1*-ssaR_{At}*, |
| | | P_{syn1}-ppc, P_{rpsU}-orfZ_{Ck}, |
| | | P_{trc}-kgdS (Synechococcus sp. |
| | | PCC 7002) |

The strains were grown in a shake plate assay to examine production of P4HB. Three replicates of each of the three strains were cultured overnight in a sterile tube containing 3 mL of LB with 50 μg/mL kanamycin and either 25 μg/mL chloramphenicol (strain 14) or 100 μg/mL ampicillin (for strains 15 and 16). Assay conditions for the shake plate experiment were the same as described in Example 1 except that 50 g/L glucose, 5 mM MgSO₄ and 10 μM IPTG was used in the medium. Parallel cultures of strains 15 and 16 were also grown where 100 μM IPTG was added as indicated in Table 12. Preparation and analysis of the cultures were carried out as described in Example 1.

As shown in Table 12, the sucD_{Ck}* expressing production strain 14 produced a P4HB titer similar to strain 15 that expressed the kgdP-M38 with 100 μM IPTG. However, moderate expression of the native kgdS by strain 16 with 10 μM IPTG clearly surpassed the P4HB production capabilities of both strains 14 and 15, demonstrating the superior performance of KgdS for P4HB production.

TABLE 12
Biomass and P4HB titer

| Strains | [IPTG] (μM) | Biomass Titer (g/L) | P4HB Titer (% dcw) |
|---|---|---|---|
| 14 | 10 | 4.94 ± 0.03 | 38.8 ± 2.5% |
| 15 | 10 | 3.75 ± 0.05 | 15.4 ± 1.9% |
| | 100 | 4.66 ± 0.05 | 32.2 ± 0.2% |
| 16 | 10 | 6.54 ± 0.02 | 53.1 ± 1.0% |
| | 100 | 5.84 ± 0.08 | 47.8 ± 0.3% |

### Example 6

### Improved P4HB Production by Expression of a Malonyl-CoA Reductase Gene

Two types of malonyl-CoA reductases were described in the literature. The malonyl-CoA reductase from Chloroflexus aurantiacus catalyzes the two-step reduction of malonyl-CoA and NADPH to 3-hydroxypropionate via malonate semialdehyde (Hugler et al., J. Bacteriol. 184(9):2404-2410 (2002)). By contrast, the malonyl-CoA reductase from Metallosphaera sedula and its homologue from Sulfolobus tokodaii are monofunctional proteins that only catalyze the conversion of malonyl-CoA to malonate semialdehyde, but not the conversion of the later to 3-hydroxypropionate (Alber et al., J. Bacteriol. 188(24):8551-8559 (2006)).

This example demonstrates that expression of the malonyl-CoA reductase gene from S. tokodaii improved P4HB production as compared to strains that did not express this gene. The following two strains were constructed, both containing chromosomal deletions in yneI, gabD, pykF and pykA and overexpressing the PHA synthase phaC3/C1*, the sucD_{Ck}* and the orfZ_{Ck} genes from C. kluyveri, the ssaR_{At}* gene from A. thaliana, and the E. coli ppc gene. Strain 17 containing these modifications served as the control for strain 18, which also expressed the mcr_{St}* gene from S. tokodaii from the P_{syn1} promoter (Table 13).

TABLE 13
Microbial Strains used in Example 6

| | Relevant host genome | |
|---|---|---|
| Strains | deletions | Genes overexpressed |
| 17 | ΔyneI, ΔgabD, ΔpykF, | Pₓ-phaC3/C1*, P_{uspA}-sucD_{Ck}*-ssaR_{At}*, |
| | ΔpykA | P_{syn1}-ppc, P_{rpsU}-orfZ_{Ck} |
| 18 | ΔyneI, ΔgabD, ΔpykF, | Pₓ-phaC3/C1*, P_{uspA}-sucD_{Ck}*-ssaR_{At}*, |
| | ΔpykA | P_{syn1}-ppc, P_{rpsU}-orfZ_{Ck}, P_{syn1}-mcr_{St}* |

Three replicates of strains 17 and 18 were cultured overnight in a sterile tube containing 3 mL of LB with either 15 μg/mL tetracycline (strain 17) or 25 μg/mL chloramphenicol (strain 18). The shake plate was grown for 5 hours at 37° C. with shaking and then incubated at 30° C. for a total of 48 hours. Assay conditions for the shake plate experiment were the same as described in Example 1 except that 30 g/L glucose was used in the medium and IPTG was not added. Preparation and analysis of the cultures were carried out as described in Example 1.

As shown in Table 14, the mcr_{St}* expressing production strain 18 surprisingly and unexpectedly produced a much higher P4HB titer as compared to control strain 17 that did not express this gene.

TABLE 14
Biomass and P4HB titer

| Strains | Biomass Titer (g/L) | P4HB Titer (% dcw) |
|---|---|---|
| 17 | 6.14 ± 0.09 | 19.40 ± 0.49 |
| 18 | 6.96 ± 0.14 | 31.23 ± 2.00 |

### Example 7

### Improved P4HB Production by Expression of an Oxidative Stress-Resistant 1,2-Propanediol Oxidoreductase

The NADH-dependent oxidoreductase FucO from E. coli was identified as an L-1,2-propanediol oxidoreductase in cells growing anaerobically on L-rhamnose as a sole source of carbon and energy (Boronat and Aguilar, J. Bacteriol. 140(2):320-306 (1979); Chin and Lin, J. Bacteriol. 157(3):828-832 (1984); Zhu and Lin, J. Bacteriol. 171(2):862-867 (1989)). This propanediol oxidoreductase converts L-lactaldehyde to L-1,2-propanediol and is only catalytically active under anaerobic conditions due to inactivation of the enzyme under aerobic conditions. However, FucO mutants with increased resistance to oxidative stress were isolated (Lu et al., J. Biol. Chem. 273(14):8308-8316 (1998)). An expanded role for FucO was demonstrated by Wang et al. (Appl. Environ. Microbiol. 77(15):5132-5140 (2011)) who showed that expression of fucO from plasmids in engineered E. coli strains substantially increased furfural tolerance by converting the toxic furfural to the less-toxic furfuryl alcohol.

This example demonstrates that expression of the E. coli fucO gene variant, hereafter called fucO_{16L-L7V}, encoding an oxidoreductase with increased resistance to oxidative stress improved P4HB production as compared to a strain that did not express this gene. The following two strains were constructed, both overexpressing the PHA synthase phaC3/C1*, the sucD_{Ck}* and the orfZ_{Ck} genes from C. kluyveri, and the E. coli ppc gene. Neither strain expressed the ssaR_{At}* gene from A. thaliana used in previous examples. Both strains contained chromosomal deletions in yneI, gabD, pykF, pykA, and fucO and also had gene knock-out mutations in the two aldehyde dehydrogenases yqhD and yihU whose gene products were shown to convert succinic semialdehyde to 4-hydroxybutyate (Van Walsem et al., U.S. Patent Application No. WO 2011100601; Saito et al., J. Biol. Chem. 284(24):16442-16451 (2009); FIG. 1, reaction 8). Strain 19 containing all these modifications served as the control for strain 20, which also expressed the fucO_{16L-L7V} from the IPTG-inducible P_{trc} promoter (Table 15).

TABLE 15
Microbial Strains used in Example 7

| | Relevant | |
|---|---|---|
| Strains | host genome deletions | Genes overexpressed |
| 19 | ΔyneI, ΔgabD, ΔpykF, | Pₓ-phaC3/C1*, P_{uspA}-sucD_{Ck}*, |
| | ΔpykA, ΔyqhD, ΔyihU, | P_{syn1}-ppc, P_{rpsU}-orfZ_{Ck} |
| | ΔfucO | |
| 20 | ΔyneI, ΔgabD, ΔpykF, | Pₓ-phaC3/C1*, P_{uspA}-sucD_{Ck}*, |
| | ΔpykA, ΔyqhD, ΔyihU, | P_{syn1}-ppc, P_{rpsU}-orfZ_{Ck}, |
| | ΔfucO | P_{trc}-fucO_{16L-L7V} |

Three replicates of strains 19 and 20 were cultured overnight in a sterile tube containing 3 mL of LB with 50 μg/mL kanamycin and 100 μg/mL ampicillin. The shake plate was grown for 5 hours at 37° C. with shaking and then incubated at 28° C. for a total of 42 hours. Assay conditions for the shake plate experiment were the same as described in Example 1 except that 40 g/L glucose was used in the medium and either 0, 10, or 100 μM IPTG was added as indicated in Table 16. Preparation and analysis of the cultures were carried out as described in Example 1.

As shown in Table 16, control strain 19 still produced significant amounts of P4HB even though it contained chromosomal gene knock-out mutations in yqhD, yihU and fucO, presumably due to one or more unidentified, endogenous succinic semialdehyde reductases. Strain 20 expressing fucO_{16L-L7V} produced a higher P4HB titer as compared to control strain 19 showing that the FucO mutant enzyme with increased resistance to oxidative stress was able to convert succinic semialdehyde to 4-hydroxybutyrate.

TABLE 16
Biomass and P4HB titer

| Strains | [IPTG] (μM) | Biomass Titer (g/L) | P4HB Titer (% dcw) |
|---|---|---|---|
| 19 | 0 | 4.43 | 34.55 |
| | 10 | 4.40 | 34.85 |
| | 100 | 4.42 | 34.89 |
| 20 | 0 | 4.67 | 33.78 |
| | 10 | 5.09 | 39.57 |
| | 100 | 5.21 | 40.81 |

### Example 8

### Improved P4HB Production by Reduced Expression of the Endogenous E. coli Succinyl-CoA Synthetase

This example demonstrates that reducing the expression of the endogenous E. coli succinyl-CoA synthetase encoded by sucCD enhances P4HB production.

The following two strains were constructed, both overexpressing the PHA synthases phaC3/C1* and phaC183*, the sucD_{Ck}* and the orfZ_{Ck} genes from C. kluyveri, the ssaR_{At}* gene from A. thaliana, and the E. coli ppc gene. Both strains contained host genome deletions in yneI, gabD, pykF and pykA and contained the fadR601 mutation that was shown to derepress the glyoxylate shunt enzymes aceB and aceA (Rhie and Dennis, Appl. Envion. Microbiol. 61(7):2487-2492 (1995)). Therefore, both strains also contained a chromosomal deletion of the aceBA operon. Strain 21 containing all these modifications served as the control for strain 22, which in addition also contained a chromosomal deletion of the sucCD genes (Table 17).

TABLE 17
Microbial Strains used in Example 8

| | Relevant | |
|---|---|---|
| Strains | host genome deletions | Genes overexpressed |
| 21 | fadR601, ΔyneI, | Pₓ-phaC3/C1*, P_{uspA}-sucD_{Ck}*-ssaR_{At}*, |
| | ΔgabD, ΔpykF, | P_{rpsU}-orfZ_{Ck}, P_{syn1}-ppc, P_{syn1}-phaC183* |
| | ΔpykA, ΔaceBA | |
| 22 | fadR601, ΔyneI, | Pₓ-phaC3/C1*, P_{uspA}-sucD_{Ck}*-ssaR_{At}*, |
| | ΔgabD, ΔpykF, | P_{rpsU}-orfZ_{Ck}, P_{syn1}-ppc, P_{syn1}-phaC183* |
| | ΔpykA, ΔaceBA, | |
| | ΔsucCD | |

Three replicates of strains 21 and 22 were cultured overnight in a sterile tube containing 3 mL of LB with 50 μg/mL kanamycin. The shake plate was grown for 5 hours at 37° C. with shaking and then incubated at 28° C. for a total of 47 hours. Assay conditions for the shake plate experiment were the same as described in Example 1 except that 45 g/L glucose was used as the sole carbon source. Preparation and analysis of the cultures were carried out as described in Example 1.

As shown in Table 18, strain 22 having reduced succinyl-CoA synthetase activity produced a higher P4HB titer than the control strain 21.

TABLE 18
Biomass and P4HB titer

| Strains | Biomass Titer (g/L) | P4HB Titer (%dcw) |
|---|---|---|
| 21 | 13.6 ± 0.1 | 60 ± 0.8% |
| 22 | 17.0 ± 0.3 | 72 ± 3.4% |

### Example 9

### Improved P4HB Production by Expression of an NADH-Dependent Fumarate Reductase

This example demonstrates that expression of a heterologous fumarate reductase gene enhances P4HB production. The reaction catalysed by endogenous fumarate reductase allows fumarate to serve as a terminal electron acceptor when E. coli is growing under anaerobic conditions. The fumarate reductase is membrane-bound and uses reduced menaquinone to convert fumarate to succinate. By contrast, the fumarate reductase from Trypanosoma brucei called FRDg is active under aerobic conditions, is soluble (i.e. not membrane-bound) and uses NADH to convert fumarate to succinate (Besteiro et al., J. Biol. Chem. 277 (41):38001-38012 (2002)). Expression of FRDg in P4HB production strains may increase PHA titers by forcing more carbon in a reverse TCA cycle carbon flux towards the P4HB pathway. To test this, the following two strains were constructed, both containing chromosomal deletions in yneI, gabD, pykF and pykA and overexpressing the PHA synthase phaC3/C1*, the sucD_{Ck}* and the orfZ_{Ck} genes from C. kluyveri, the ssaR_{At}* gene from A. thaliana, and the E. coli ppc gene. Strain 23 containing these modifications served as the control for strain 24, which also expressed the frd_g* gene from T. brucei from the P_{trc} promoter (Table 19).

TABLE 19
Microbial Strains used in Example 9

| | Relevant | |
|---|---|---|
| Strains | host genome deletions | Genes overexpressed |
| 23 | ΔyneI, ΔgabD, | Pₓ-phaC3/C1*, P_{uSpA}-sucD_{Ck}*-ssaR_{At}*, |
| | ΔpykF, ΔpykA | P_{syn1}-ppc, P_{rpsU}-orfZ_{Ck} |
| 24 | ΔyneI, ΔgabD, | Pₓ-phaC3/C1*, P_{uspA}-sucD_{Ck}*-ssaR_{At}*, |
| | ΔpykF, ΔpykA | P_{syn1}-ppc, P_{rpsU}-orfZ_{Ck}, P_{trc}-frd_g* |

Three replicates of strains 23 and 24 were cultured overnight in a sterile tube containing 3 mL of LB with 15 μg/mL tetracycline and 100 μg/mL ampicillin. The shake plate was grown for 5 hours at 37° C. with shaking and then incubated at 30° C. for a total of 24 hours. Assay conditions for the shake plate experiment were the same as described in Example 1 except that 20 g/L glucose was used as the sole carbon source. Parallel cultures of strains 23 and 24 were also grown where either 0 μM or 100 μM IPTG was added. Preparation and analysis of the cultures were carried out as described in Example 1.

As shown in Table 20, strain 24 expressing the frd_g* gene from T. brucei produced a higher P4HB titer than control strain 23.

TABLE 20
Biomass and P4HB titer

| Strains | [IPTG] (μM) | Biomass Titer (g/L) | P4HB Titer (% dcw) |
|---|---|---|---|
| 23 | 0 | 4.67 ± 0.03 | 37.68 ± 0.34 |
| | 100 | 4.64 ± 0.13 | 39.43 ± 0.52 |
| 24 | 0 | 4.78 ± 0.09 | 37.65 ± 0.83 |
| | 100 | 5.59 ± 0.09 | 47.52 ± 0.68 |

The T. brucei FRDg enzyme is 1142 amino acid long and is a putative multifunctional protein composed of three different domains. The N-terminal domain (from position 37 to 324) is homologous to the ApbE protein possibly involved in thiamine biosynthesis, the C-terminal domain is homologous to cytochrome b₅ reductases and the cytochrome domain of nitrate reductases (from position 906 to 1128), and the central domain is homologous to fumarate reductases (Besteiro et al., J. Biol. Chem. 277 (41):38001-38012 (2002)). Thus, expression of the central domain of FRDg only is expected to be sufficient to obtain the observed P4HB titer increase in this Example.

### Example 10

### Improved P4HB Production by Expression of a Pyruvate Carboxylase Gene

This example demonstrates that expression of a heterologous pyruvate carboxylase gene improved P4HB production as compared to a strain that did not express this gene. The following two strains were constructed, both containing chromosomal deletions in yneI, gabD and overexpressing the PHA synthase phaC3/C1*, the sucD_{Ck}* and the orfZ_{Ck} genes from C. kluyveri, the ssaR_{At}* gene from A. thaliana, and the E. coli ppc gene. Strain 25 containing these modifications served as the control for strain 26, which also expressed the pyc_{Ll} gene from L. lactis from the P_{trc} promoter (Table 21).

TABLE 21
Microbial Strains used in Example 10

| | Relevant host | |
|---|---|---|
| Strains | genome deletions | Genes overexpressed |
| 25 | ΔyneI ΔgabD | Pₓ-phaC3/C1*, P_{uspA}-sucD_{Ck}*-ssaR_{At}*, |
| | | P_{syn1}-ppc, P_{rpsU}-orfZ_{Ck} |
| 26 | ΔyneI ΔgabD | Pₓ-phaC3/C1*, P_{uspA}-sucD_{Ck}*-ssaR_{At}*, |
| | | P_{syn1}-ppc, PP_{rpsU}orfZ_{Ck}, P_{trc}-pyc_{Ll} (L. lactis) |

Three replicates of strains 25 and 26 were cultured overnight in a sterile tube containing 3 mL of LB with 25 μg/mL chloramphenicol and 100 μg/mL ampicillin. The shake plate was grown for 5 hours at 37° C. with shaking and then incubated at 30° C. for a total of 48 hours. Assay conditions for the shake plate experiment were the same as described in Example 1 except that 20 g/L glucose was used as the sole carbon source. Parallel cultures of strains 25 and 26 were grown where either 0, 50, 150 or 250 μM IPTG was added. Preparation and analysis of the cultures were carried out as described in Example 1.

As shown in Table 22, strain 26 that expressed the pyc_{Ll} gene from L. lactis produced a higher P4HB titer than control strain 25.

TABLE 22
Biomass and P4HB titer

| Strain | [IPTG] (μM) | Biomass Titer (g/L) | P4HB Titer (% dcw) |
|---|---|---|---|
| 25 | 0 | 5.12 | 45.72 |
| | 50 | 4.90 | 45.03 |
| | 150 | 4.89 | 44.67 |
| | 250 | 5.02 | 44.61 |
| 26 | 0 | 5.49 | 47.10 |
| | 50 | 6.93 | 55.26 |
| | 150 | 6.77 | 55.35 |
| | 250 | 6.37 | 53.38 |

### Example 11

### Improved P4HB Production by Expression of an NADH Kinase Gene

This example demonstrates that expression of a heterologous NADH kinase gene improved P4HB production as compared to a strain that did not express this gene. Expression of such NADH kinase genes is expected to result in increased intracellular NADPH concentrations, which are used for high level production of P4HB because two 4HB pathway enzymes, encoded by sucD_{Ck}* and ssaR_{At}*, require this reducing equivalent. To test this, the ndk_{An}* gene from Aspergillus nidulans encoding the NADH kinase, a.k.a. ATP:NADH 2′ phosphotransferase (Panagiotou et al., Metabol. Engin. 11:31-39 (2009)) was overspressed. The following two strains were constructed, both containing chromosomal deletions in yneI and gabD and overexpressing the PHA synthase phaC1, the sucD_{Ck}* and the orfZ_{Ck} genes from C. kluyveri, and the ssaR_{At}* gene from A. thaliana. Strain 27 containing these modifications served as the control for strain 28, which also expressed the ndk_{An}* gene from A. nidulans from the P_{trc} promoter (Table 23).

TABLE 23
Microbial Strains used in Example 11

| | Relevant host genome | |
|---|---|---|
| Strains | deletions | Genes overexpressed |
| 27 | ΔyneI ΔgabD | Pₓ-P_{syn1}-phaC1, P_{uspA}-sucD_{Ck}*-ssaR_{At*}, |
| | | P_{rpsU}-orfZ_{Ck}, |
| 28 | ΔyneI ΔgabD | Pₓ-P_{syn1}-phaC1, P_{uspA}-sucD_{Ck}*-ssaR_{At*}, |
| | | P_{rpsU}-orfZ_{Ck}, P_{trc}-ndk_{An}* |

Three replicates of strains 27 and 28 were cultured overnight in a sterile tube containing 3 mL of LB with 25 μg/mL chloramphenicol and 100 μg/mL ampicillin. The shake plate was grown for 5 hours at 37° C. with shaking and then incubated at 30° C. for a total of 48 hours. Assay conditions for the shake plate experiment were the same as described in Example 1 except that 25 g/L glucose was used as the sole carbon source. Preparation and analysis of the cultures were carried out as described in Example 1.

As shown in Table 24, strain 28 expressing the ndk_{An}* gene from A. nidulans produced a significantly higher P4HB titer than control strain 27.

TABLE 24
Biomass and P4HB titer

| Strain | Biomass Titer (g/L) | P4HB Titer (% dcw) |
|---|---|---|
| 27 | 2.9 ± 0.1 | 11.5 ± 1.7 |
| 28 | 5.9 ± 0.2 | 34.8 ± 4.6 |

### Example 12

### Improved P4HB Production by Addition of Pantothenate to Fermentation Media

This example shows that addition of pantothenate to the fermentation media improved P4HB production as compared to a fermentation medium that did not contain this metabolite. Fed pantothenate is taken up by E. coli using the pantothenate:Na⁺ symporter encoded by panF (Jackowski and Alix, J. Bacteriol. 172(7):3842-8 (1990); FIG. 1). Pantothenate is a metabolic precursor of coenzyme A which can be converted to acetyl-CoA by acetyl-CoA synthetase (E.C. 6.2.1.1.) in the following reaction (1):

acetate+ATP+coenzyme A→acetyl-CoA+AMP+diphosphate (1)

Addition of pantothenate to the fermentation media may improve P4HB production by increasing the intracellular acetyl-CoA pool needed to replenish the TCA cycle and/or converting the acetate formed by the CoA transferase encoded by the orfZ_{Ck} from C. kluyveri in the following reaction (2):

4-hydroxybutyrate+acetyl-CoA→4-hydroxybutyryl-CoA+acetate (2)

To test this, strain 29 was used that contained chromosomal deletions in yneI, gabD, pykF and pykA and overexpressed the PHA synthase phaC3/C1*, the sucD_{Ck}* and the orfZ_{Ck} genes from C. kluyveri, the ssaR_{At}* gene from A. thaliana, and the E. coli ppc gene (Table 25).

TABLE 25
Microbial Strain used in Example 12

| | Relevant host | |
|---|---|---|
| Strain | genom edeletions | Genes overexpressed |
| 29 | ΔyneI, ΔgabD, ΔpykF, | Pₓ-phaC3/C1*-P_{uspA}-sucD_{Ck}*-ssaR_{At}*, |
| | ΔpykA | P_{rpsU}-orfZ_{Ck}, P_{syn1}-ppc |

Three replicates of strains 29 were cultured overnight in a sterile tube containing 3 mL of LB with 25 μg/mL chloramphenicol. The shake plate was grown for 6 hours at 37° C. with shaking and then incubated at 28° C. for a total of 46 hours. Assay conditions for the shake plate experiment were the same as described in Example 1 except that 43.5 g/L glucose was used with either 0 or 5 mM pantothenate supplemented in the medium. At the end of the growth phase, the biomass and P4HB titers were determined as outlined in Example 1.

As shown in Table 26, addition of 5 mM pantothenate produced a higher P4HB titer than when no pantothenate was added to the fermentation media.

TABLE 26
Biomass and P4HB titer of strain 29 with pantothenate supplementation

| Pantothenate (mM) | Biomass Titer (g/L) | P4HB Titer (% dcw) |
|---|---|---|
| 0 | 5.40 ± 0.13 | 46.6 ± 0.2 |
| 5 | 6.15 ± 0.09 | 50.4 ± 0.4 |

Gene ID 001 Nucleotide Sequence: Lactococcus lactis subsp. Lactis Berridge X 13 pyruvate carboxylase pyc_{Ll}

(SEQ ID NO: 19)
ATGAAAAAACTACTCGTCGCCAATCGTGGAGAAATCGCCGTTCGTGTCTT
TCGTGCCTGTAATGAACTCGGACTTTCTACAGTAGCCGTCTATGCAAGAG
AAGATGAATATTCCGTTCATCGCTTTAAAGCAGATGAATCTTACCTTATC
GGTCAAGGTAAAAAACCAATTGATGCTTATTTGGATATTGATGATATTAT
TCGTGTTGCTCTTGAATCAGGAGCAGATGCCATTCATCCCGGTTATGGTC
TTTTATCTGAAAATCTTGAATTTGCTACAAAAGTTCGAGCAGCAGGATTA
GTTTTTGTCGGTCCTGAACTTCATCATTTGGATATTTTCGGCGATAAAAT
CAAAGCAAAAGCCGCAGCTGATGAAGCTCAAGTTCCCGGAATTCCCGGAA
CAAATGGTGCAGTAGATATTGACGGAGCTCTTGAATTTGCTCAAACTTAC
GGATATCCAGTCATGATTAAGGCAGCATTGGGCGGCGGCGGTCGTGGAAT
GCGTGTTGCGCGTAATGACGCTGAAATGCACGACGGATATGCTCGTGCGA
AATCAGAAGCTATCGGTGCCTTTGGTTCTGGAGAAATCTATGTTGAAAAA
TACATTGAAAATCCTAAGCATATTGAAGTTCAAATTCTTGGGGATAGTCA
TGGAAATATTGTCCATTTGCACGAACGTGATTGCTCTGTCCAACGCCGAA
ATCAAAAAGTCATTGAAATTGCTCCAGCCGTAGGACTCTCACCAGAGTTC
CGTAATGAAATTTGTGAAGCAGCAGTTAAACTTTGTAAAAATGTTGGCTA
TGTTAATGCTGGGACGGTTGAATTTTTAGTCAAAGATGATAAGTTCTACT
TTATCGAAGTCAACCCACGTGTTCAAGTTGAACACACAATTACCGAGCTT
ATTACAGGTGTAGATATTGTTCAAGCACAAATTTTGATTGCTCAAGGCAA
AGATTTACATACAGAAATTGGTATCCCGGCACAAGCTGAAATACCACTTT
TGGGCTCAGCCATTCAATGTCGTATTACTACAGAAGACCCGCAAAATGGC
TTCTTGCCAGATACAGGTAAAATCGATACCTACCGTTCACCAGGTGGTTT
CGGCATTCGTTTGGACGTTGGAAATGCCTATGCTGGTTATGAAGTGACTC
CCTATTTTGACTCGCTTTTAGTAAAAGTTTGTACCTTTGCTAATGAATTT
AGCGATAGTGTACGTAAAATGGATCGTGTGCTTCATGAATTCCGTATTCG
TGGGGTGAAAACTAATATTCCATTTTTGATTAATGTTATTGCCAATGAAA
ACTTTACGAGCGGACAAGCAACAACAACCTTTATTGACAATACTCCAAGT
CTTTTCAATTTCCCACGCTTACGTGACCGTGGAACAAAAACCTTACACTA
CTTATCAATGATTACAGTCAATGGTTTCCCAGGGATTGAAAATACAGAAA
AACGCCATTTTGAAGAACCTCGTCAACCTCTACTTAACATTGAAAAGAAA
AAGACAGCTAAAAATATCTTAGATGAACAAGGGGCTGATGCGGTAGTTGA
ATATGTGAAAAATACAAAAGAAGTATTATTGACAGATACAACTTTACGTG
ATGCTCACCAGTCTCTTCTTGCCACTCGTTTGCGTTTGCAAGATATGAAA
GGAATTGCTCAAGCCATTGACCAAGGACTTCCAGAACTTTTCTCAGCTGA
AATGTGGGGTGGGGCAACCTTTGATGTCGCTTATCGTTTCTTGAATGAAT
CGCCTTGGTATCGTCTACGTAAATTACGTAAACTCATGCCAAATACCATG
TTCCAAATGCTTTTCCGTGGTTCAAATGCAGTTGGATATCAAAACTATCC
TGATAATGTCATTGAAGAATTTATCCACGTAGCTGCACATGAAGGAATCG
ATGTCTTTCGTATCTTTGATAGCCTCAACTGGTTGCCACAAATGGAAAAA
TCAATCCAAGCAGTGCGTGATAATGGAAAAATTGCCGAAGCAACCATTTG
TTATACAGGAGATATCCTTGACCCAAGTCGACCAAAATATAATATCCAAT
ACTACAAAGATTTGGCAAAAGAGTTAGAAGCTACTGGGGCTCATATACTT
GCCGTTAAAGATATGGCGGGCTTGTTGAAACCTCAAGCGGCATATCGCTT
GATTTCAGAATTAAAAGATACGGTTGACTTACCAATTCACTTGCATACAC
ATGATACTTCAGGAAATGGTATTATTACCTATTCTGGTGCAACTCAAGCA
GGAGTAGATATTATTGATGTGGCAACTGCCAGTCTTGCTGGTGGAACTTC
TCAACCTTCAATGCAATCAATTTATTATGCCCTTGAACATGGTCCCCGTC
ATGCTTCAATTAATGTGAAAAATGCAGAGCAAATTGACCATTATTGGGAA
GATGTGCGTAAATATTATGCACCTTTTGAGGCAGGAATTACGAGCCCACA
AACTGAAGTTTACATGCATGAAATGCCTGGCGGACAATATACTAACTTGA
AATCTCAAGCAGCAGCTGTTGGACTTGGACATCGTTTTGATGAAATCAAA
CAAATGTATCGTAAAGTAAACATGATGTTTGGCGATATCATTAAAGTAAC
TCCTTCATCAAAAGTAGTTGGTGATATGGCACTCTTTATGATTCAAAACG
AATTGACAGAAGAGGATGTCTATGCGCGAGGAAATGAGCTTAACTTCCCT
GAATCAGTAGTCTCATTCTTCCGTGGTGATTTAGGACAGCCTGTTGGAGG
TTTCCCAGAAGAACTACAAAAAATTATTGTAAAAGACAAATCGGTCATTA
TGGATCGTCCAGGATTACATGCCGAAAAAGTTGATTTTGCAACTGTAAAA
GCTGACTTGGAACAAAAAATTGGTTATGAACCAGGTGATCATGAAGTTAT
CTCTTACATTATGTATCCACAAGTTTTCCTTGATTATCAAAAAATGCAAA
GAGAATTTGGAGCTGTCACACTACTCGATACTCCAACTTTCTTACACGGA
ATGCGCCTCAATGAAAAAATTGAAGTCCAAATTGAAAAAGGTAAAACGCT
CAGCATTCGTTTAGATGAAATAGGAGAACCTGACCTCGCTGGAAATCGTG
TGCTCTTCTTTAACTTGAACGGTCAGCGTCGTGAAGTTGTTATTAATGAC
CAATCCGTTCAAACTCAAATTGTAGCTAAACGTAAGGCCGAAACAGGTAA
TCCAAACCAAATTGGAGCAACTATGCCCGGTTCTGTTCTTGAAATCCTAG
TTAAAGCTGGAGATAAAGTTAAAAAAGGACAAGCTTTGATGGTTACTGAA
GCCATGAAGATGGAAACGACCATTGAGTCACCATTTGATGGAGAGGTTAT
TGCCCTTCATGTTGTCAAAGGTGAAGCCATTCAAACACAAGACTTATTGA
TTGAAATTGACTAA

Gene ID 001 Amino Acid Sequence: Lactococcus lactis subsp. Lactis Berridge X 13 pyruvate carboxylase Pyc_{Ll}

(SEQ ID NO: 20)
MKKLLVANRGEIAVRVFRACNELGLSTVAVYAREDEYSVHRFKADESYLI
GQGKKPIDAYLDIDDIIRVALESGADAIHPGYGLLSENLEFATKVRAAGL
VFVGPELHHLDIFGDKIKAKAAADEAQVPGIPGTNGAVDIDGALEFAQTY
GYPVMIKAALGGGGRGMRVARNDAEMHDGYARAKSEAIGAFGSGEIYVEK
YIENPKHIEVQILGDSHGNIVHLHERDCSVQRRNQKVIEIAPAVGLSPEF
RNEICEAAVKLCKNVGYVNAGTVEFLVKDDKFYFIEVNPRVQVEHTITEL
ITGVDIVQAQILIAQGKDLHTEIGIPAQAEIPLLGSAIQCRITTEDPQNG
FLPDTGKIDTYRSPGGFGIRLDVGNAYAGYEVTPYFDSLLVKVCTFANEF
SDSVRKMDRVLHEFRIRGVKTNIPFLINVIANENFTSGQATTTFIDNTPS
LFNFPRLRDRGTKTLHYLSMITVNGFPGIENTEKRHFEEPRQPLLNIEKK
KTAKNILDEQGADAVVEYVKNTKEVLLTDTTLRDAHQSLLATRLRLQDMK
GIAQAIDQGLPELFSAEMWGGATFDVAYRFLNESPWYRLRKLRKLMPNTM
FQMLFRGSNAVGYQNYPDNVIEEFIHVAAHEGIDVFRIFDSLNWLPQMEK
SIQAVRDNGKIAEATICYTGDILDPSRPKYNIQYYKDLAKELEATGAHIL
AVKDMAGLLKPQAAYRLISELKDTVDLPIHLHTHDTSGNGIITYSGATQA
GVDIIDVATASLAGGTSQPSMQSIYYALEHGPRHASINVKNAEQIDHYWE
DVRKYYAPFEAGITSPQTEVYMHEMPGGQYTNLKSQAAAVGLGHRFDEIK
QMYRKVNMMFGDIIKVTPSSKVVGDMALFMIQNELTEEDVYARGNELNFP
ESVVSFFRGDLGQPVGGFPEELQKIIVKDKSVIMDRPGLHAEKVDFATVK
ADLEQKIGYEPGDHEVISYIMYPQVFLDYQKMQREFGAVTLLDTPTFLHG
MRLNEKIEVQIEKGKTLSIRLDEIGEPDLAGNRVLFFNLNGQRREVVIND
QSVQTQIVAKRKAETGNPNQIGATMPGSVLEILVKAGDKVKKGQALMVTE
AMKMETTIESPFDGEVIALHVVKGEAIQTQDLLIEID

Gene ID 002 Nucleotide Sequence: Sulfolobus tokodaii malonyl-CoA reductase mcr_{St}

(SEQ ID NO: 21)
ATGATCCTGATGCGCCGCACCCTCAAAGCAGCAATCCTGGGCGCCACGGG
CTTGGTTGGTATTGAGTACGTGCGCATGCTGAGCAATCACCCGTATATCA
AACCAGCATATCTGGCGGGTAAGGGCAGCGTTGGCAAGCCTTACGGTGAG
GTCGTGCGCTGGCAGACGGTAGGTCAGGTGCCGAAAGAAATTGCGGACAT
GGAGATCAAGCCGACGGACCCGAAGCTGATGGATGACGTTGACATTATCT
TCTCCCCGCTGCCGCAGGGTGCAGCTGGTCCGGTGGAAGAACAATTTGCC
AAAGAAGGTTTTCCTGTTATTAGCAACAGCCCGGACCATCGCTTTGATCC
GGACGTTCCGCTGCTGGTGCCGGAGCTGAATCCGCATACGATCAGCTTGA
TTGACGAGCAACGTAAGCGTCGCGAGTGGAAAGGTTTTATCGTCACTACG
CCGCTGTGCACCGCCCAAGGTGCGGCCATTCCGCTGGGCGCAATCTTCAA
AGATTACAAGATGGACGGTGCGTTTATCACCACCATCCAGAGCCTGAGCG
GCGCTGGCTATCCGGGTATTCCGTCCCTGGATGTGGTTGATAACATTCTG
CCGCTGGGCGATGGTTACGACGCCAAGACCATTAAAGAAATCTTCCGTAT
CCTGAGCGAGGTTAAACGTAATGTTGACGAGCCGAAACTGGAGGATGTGT
CTCTGGCGGCGACCACGCACCGTATCGCGACCATTCACGGTCATTACGAA
GTCCTGTATGTGAGCTTCAAAGAAGAAACTGCAGCGGAGAAGGTCAAAGA
AACCCTGGAGAACTTCCGTGGCGAGCCTCAGGATTTGAAGTTGCCGACCG
CGCCATCGAAACCGATTATTGTCATGAACGAAGATACCCGTCCGCAGGTT
TACTTCGACCGTTGGGCGGGTGATATCCCGGGTATGAGCGTTGTCGTCGG
TCGTCTGAAGCAAGTGAACAAGCGTATGATTCGTCTGGTTAGCCTGATTC
ACAATACCGTGCGTGGCGCTGCGGGTGGTGGCATCCTGGCAGCGGAGCTG
TTGGTCGAGAAAGGCTATATTGAAAAGTAA

Gene ID 002 Amino Acid Sequence: Sulfolobus tokodaii malonyl-CoA reductase Mcr_{St}

(SEQ ID NO: 22)
MILMRRTLKAAILGATGLVGIEYVRMLSNHPYIKPAYLAGKGSVGKPYGE
VVRWQTVGQVPKEIADMEIKPTDPKLMDDVDIIFSPLPQGAAGPVEEQFA
KEGFPVISNSPDHRFDPDVPLLVPELNPHTISLIDEQRKRREWKGFIVTT
PLCTAQGAAIPLGAIFKDYKMDGAFITTIQSLSGAGYPGIPSLDVVDNIL
PLGDGYDAKTIKEIFRILSEVKRNVDEPKLEDVSLAATTHRIATIHGHYE
VLYVSFKEETAAEKVKETLENFRGEPQDLKLPTAPSKPIIVMNEDTRPQV
YFDRWAGDIPGMSVVVGRLKQVNKRMIRLVSLIHNTVRGAAGGGILAAEL
LVEKGYIEK

Gene ID 003 Nucleotide Sequence: Pseudonocardia dioxanivorans CB 1190 alpha-ketoglutarate reductase kgdP-M38

(SEQ ID NO: 23)
ATGTCCACCAGCAGTACCTCCGGCCAGACGAGCCAGTTCGGCCCCAACGA
ATGGCTCGTCGAGGAGATGTACCAGCGTTTCCTCGACGACCCGGATGCCG
TCGACGCCGCCTGGCACGACTTCTTCGCCGACTACCGGCCGCCGTCCGGT
GACGACGAGACGGAGTCGAACGGAACCACCTCCACCACGACGACCCCGAC
CGCCTCCGCGTCCGCCGCCGCTCCCCGTTCCGCCGCCGCCTCCGGGACGG
CCGCGGCGAACGGCTCGGCGCCGGCCCCCGAGGACAAGGCGGAGAAGACC
ACCGAGAAGACCGTGCAGCAGCCCGCCACGCAGAAGCCGGCCCAGCAGGC
CGACCGGTCGGCGAACGGCGCCGCCCCCGGCAAGCCCGTCGCGGGCACCA
CGTCGCGTGCCGCCAAGCCCGCGCCCGCCGCCGCCGAGGGCGAGGTGCTG
CCCCTGCGCGGGGCGGCGAACGCCGTCGTCAAGAACATGAACGCCTCGCT
CGCCGTGCCGACCGCGACGAGCGTGCGCGCCGTGCCGGCGAAGCTCATCG
CCGACAACCGCATCGTCATCAACAACCAGCTCAAGCGCACGCGTGGCGGC
AAGCTGTCGTTCACCCACCTCATCGGCTACGCGGTGGTCAAGGCGCTGGC
CGACTTCCCGGTGATGAACCGGCACTTCGTCGAGGTCGACGGGAAACCCA
CCGCCGTCCAGCCGGAGCACGTCAACCTCGGCCTCGCGATCGACCTGCAG
GGCAAGAACGGGCAGCGTTCCCTCGTCGTCGTGTCGATCAAGGGCTGCGA
GGAGATGACCTTCGCGCAGTTCTGGTCCGCCTACGAGAGCATGGTCCACA
AGGCGCGCAACGGCACGCTCGCCGCCGAGGACTTCGCGGGCACCACGATC
AGCCTCACCAACCCGGGCACCCTCGGCACCAACCACTCGGTGCCGCGGTT
GATGCAGGGCCAGGGCACGATCGTCGGTGTCGGCGCGATGGAGTACCCCG
CCGAGTTCCAGGGCGCCAGCGAGGAGCGGCTCGCCGAGCTCGGCATCAGC
AAGATCATCACGCTGACGTCGACCTACGACCACCGGATCATCCAGGGCGC
GGAGTCGGGCGACTTCCTGCGCCGGGTCCACCACCTGCTGCTGGGCGGCG
ACGGGTTCTTCGACGACATCTTCCGCTCCCTGCGCGTCCCGTACGAGCCG
ATCCGCTGGGTGCAGGACTTCGCCGAGGGCGAGGTCGACAAGACCGCGCG
CGTCCTCGAGCTGATCGAGTCCTACCGCACGCGCGGCCACCTGATGGCCG
ACACCGACCCGCTCAACTACCGCCAGCGCCGTCACCCCGACCTCGACGTG
CTCAGCCACGGGCTGACGCTGTGGGACCTCGACCGCGAGTTCGCGGTCGG
CGGCTTCGCGGGCCAGCTGCGGATGAAGCTGCGCGACGTGCTCGGTGTGC
TGCGCGACGCGTACTGCCGCACCATCGGCACCGAGTACATGCACATCGCC
GACCCGGAGCAGCGGGCCTGGCTGCAGGAGCGCATCGAGGTCCCGCACCA
GAAGCCGCCGGTCGTCGAGCAGAAGTACATCCTGTCGAAGCTCAACGCCG
CCGAGGCGTTCGAGACCTTCCTGCAGACGAAGTACGTCGGGCAGAAGCGG
TTCTCCCTGGAGGGCGGCGAGACCGTCATCCCGCTGCTCGACGCCGTGCT
GGACAAGGCTGCCGAGCACGAGCTCGCCGAGGTCGTCATCGGCATGCCGC
ACCGCGGCCGGCTCAACGTGCTGGCCAACATCGTCGGCAAGCCGATCAGC
CAGATCTTCCGCGAGTTCGAGGGCAACCTCGACCCGGGCCAGGCCCACGG
CTCCGGCGACGTCAAGTACCACCTCGGCGCCGAGGGCAAGTACTTCCGCA
TGTTCGGCGACGGCGAGACGGTCGTGTCGCTGGCGTCCAACCCGAGCCAC
CTCGAGGCCGTCGACCCCGTGCTCGAGGGGATCGTCCGGGCCAAGCAGGA
CCTGCTCGACCAGGGCGACGGCGCCTTCCCGGTGCTGCCCCTGATGCTGC
ACGGCGACGCCGCGTTCGCCGGGCAGGGCGTCGTGGCCGAGACGCTGAAC
CTCGCCCTGCTGCGCGGCTACCGCACCGGCGGCACCGTGCACGTCGTCGT
CAACAACCAGGTCGGGTTCACCACCGCGCCCGAGCAGTCGCGCTCGTCGC
AGTACTGCACCGACGTCGCGAAGATGATCGGCGCGCCGGTCTTCCACGTG
AACGGCGACGACCCCGAGGCGTGCGTGTGGGTCGCCAAGCTGGCGGTCGA
GTACCGCGAGCGCTGGAACAACGACGTCGTGATCGACATGATCTGCTACC
GGCGCCGCGGCCACAACGAGGGCGACGACCCCTCGATGACGCAGCCGGCG
ATGTACGACGTCATCGACGCCAAGCGCAGCGTCCGCAAGATCTACACCGA
GTCCCTGATCGGCCGCGGCGACATCACCGTCGACGAGGCCGAGGCCGCGC
TGAAGGACTTCTCCAACCAGCTCGAGCACGTGTTCAACGAGGTCCGCGAG
CTGGAGCGCACGCCGCCGACGCTCTCGCCCTCGGTCGAGAACGAGCAGTC
GGTGCCCACCGACCTCGACACCTCGGTGCCGCTGGAGGTCATCCACCGCA
TCGGCGACACCCACGTGCAGCTGCCGGAAGGCTTCACCGTGCACCAGCGG
GTCAAGCCGGTGCTGGCCAAGCGGGAGAAGATGTCGCGCGAGGGCGACGT
CGACTGGGCCTTCGGCGAGCTGCTCGCCATGGGCTCGCTGGCGCTCAACG
GCAAGCTGGTCCGGCTCTCCGGGCAGGACTCGCGGCGCGGCACGTTCGTG
CAGCGGCACTCGGTCGTCATCGACCGCAAGACCGGCGAGGAGTACTTCCC
GCTGCGCAACCTCGCCGAGGACCAGGGCCGCTTCCTGCCCTACGACTCGG
CGCTGTCGGAGTACGCGGCGCTCGGCTTCGAGTACGGCTACTCCGTGGCC
AACCCGGACGCGCTCGTCATGTGGGAGGCGCAGTTCGGCGACTTCGTCAA
CGGCGCCCAGTCGATCATCGACGAGTTCATCTCCTCCGGTGAGGCCAAGT
GGGGGCAGATGGCCGACGTCGTGCTGCTGCTGCCGCACGGCCTCGAGGGC
CAGGGCCCCGACCACAGCTCCGGACGCATCGAGCGGTTCCTGCAGCTGTG
TGCCGAGGGGTCGATGACGGTCGCGATGCCGTCGGAGCCCGCGAACCACT
TCCACCTGCTGCGCCGGCACGCCCTCGACGGGGTGCGCCGCCCGCTGGTG
GTATTCACGCCGAAGTGGATGCTGCGCGCCAAGCAGGTCGTCAGCCCGCT
GTCGGACTTCACCGGTGGCCGCTTCCGCACCGTGATCGACGACCCGCGCT
TCCGCAACTCCGACAGCCCCGCCCCCGGGGTGCGCCGGGTGCTGCTGTGC
TCGGGCAAGATCTACTGGGAGCTGGCGGCGGCGATGGAGAAGCGCGGCGG
GCGCGACGACATCGCGATCGTCCGCATCGAGCAGCTCTACCCGGTGCCCG
ACCGCCAGCTCGCCGCGGTCCTCGAGCGCTACCCCAACGCCGACGACATC
CGCTGGGTCCAGGAGGAGCCGGCCAACCAGGGCGCGTGGCCGTTCTTCGG
CCTCGACCTGCGGGAGAAGCTCCCGGAGCGGCTCTCGGGCCTGACCCGCG
TGTCGCGGCGCCGGATGGCCGCGCCCGCGGCCGGCTCGTCGAAGGTCCAC
GAGGTCGAGCAGGCCGCGATCCTCGACGAGGCGCTGAGCTGA

Gene ID 003 Amino Acid Sequence: Pseudonocardia dioxanivorans CB 1190 alpha-ketoglutarate reductase kgdP-M38

(SEQ ID NO: 24)
MSTSSTSGQTSQFGPNEWLVEEMYQRFLDDPDAVDAAWHDFFADYRPPS
GDDETESNGTTSTTTTPTASASAAAPRSAAASGTAAANGSAPAPEDKAE
KTTEKTVQQPATQKPAQQADRSANGAAPGKPVAGTTSRAAKPAPAAAEG
EVLPLRGAANAVVKNMNASLAVPTATSVRAVPAKLIADNRIVINNQLKR
TRGGKLSFTHLIGYAVVKALADFPVMNRHFVEVDGKPTAVQPEHVNLGL
AIDLQGKNGQRSLVVVSIKGCEEMTFAQFWSAYESMVHKARNGTLAAED
FAGTTISLTNPGTLGTNHSVPRLMQGQGTIVGVGAMEYPAEFQGASEER
LAELGISKIITLTSTYDHRIIQGAESGDFLRRVHHLLLGGDGFFDDIFR
SLRVPYEPIRWVQDFAEGEVDKTARVLELIESYRTRGHLMADTDPLNYR
QRRHPDLDVLSHGLTLWDLDREFAVGGFAGQLRMKLRDVLGVLRDAYCR
TIGTEYMHIADPEQRAWLQERIEVPHQKPPVVEQKYILSKLNAAEAFET
FLQTKYVGQKRFSLEGGETVIPLLDAVLDKAAEHELAEVVIGMPHRGRL
NVLANIVGKPISQIFREFEGNLDPGQAHGSGDVKYHLGAEGKYFRMFGD
GETVVSLASNPSHLEAVDPVLEGIVRAKQDLLDQGDGAFPVLPLMLHGD
AAFAGQGVVAETLNLALLRGYRTGGTVHVVVNNQVGFTTAPEQSRSSQY
CTDVAKMIGAPVFHVNGDDPEACVWVAKLAVEYRERWNNDVVIDMICYR
RRGHNEGDDPSMTQPAMYDVIDAKRSVRKIYTESLIGRGDITVDEAEAA
LKDFSNQLEHVFNEVRELERTPPTLSPSVENEQSVPTDLDTSVPLEVIH
RIGDTHVQLPEGFTVHQRVKPVLAKREKMSREGDVDWAFGELLAMGSLA
LNGKLVRLSGQDSRRGTFVQRHSVVIDRKTGEEYFPLRNLAEDQGRFLP
YDSALSEYAALGFEYGYSVANPDALVMWEAQFGDFVNGAQSIIDEFISS
GEAKWGQMADVVLLLPHGLEGQGPDHSSGRIERFLQLCAEGSMTVAMPS
EPANHFHLLRRHALDGVRRPLVVFTPKWMLRAKQVVSPLSDFTGGRFRT
VIDDPRFRNSDSPAPGVRRVLLCSGKIYWELAAAMEKRGGRDDIAIVRI
EQLYPVPDRQLAAVLERYPNADDIRWVQEEPANQGAWPFFGLDLREKLP
ERLSGLTRVSRRRMAAPAAGSSKVHEVEQAAILDEALS

Gene ID 004 Nucleotide Sequence: Escherichia coli 1,2-propanediol oxidoreductase (resistant to oxidative stress) fucO_{I6L-L7V}

(SEQ ID NO: 25)
ATGATGGCTAACAGAATGCTGGTGAACGAAACGGCATGGTTTGGTCGGG
GTGCTGTTGGGGCTTTAACCGATGAGGTGAAACGCCGTGGTTATCAGAA
GGCGCTGATCGTCACCGATAAAACGCTGGTGCAATGCGGCGTGGTGGCG
AAAGTGACCGATAAGATGGATGCTGCAGGGCTGGCATGGGCGATTTACG
ACGGCGTAGTGCCCAACCCAACAATTACTGTCGTCAAAGAAGGGCTCGG
TGTATTCCAGAATAGCGGCGCGGATTACCTGATCGCTATTGGTGGTGGT
TCTCCACAGGATACTTGTAAAGCGATTGGCATTATCAGCAACAACCCGG
AGTTTGCCGATGTGCGTAGCCTGGAAGGGCTTTCCCCGACCAATAAACC
CAGTGTACCGATTCTGGCAATTCCTACCACAGCAGGTACTGCGGCAGAA
GTGACCATTAACTACGTGATCACTGACGAAGAGAAACGGCGCAAGTTTG
TTTGCGTTGATCCGCATGATATCCCGCAGGTGGCGTTTATTGACGCTGA
CATGATGGATGGTATGCCTCCAGCGCTGAAAGCTGCGACGGGTGTCGAT
GCGCTCACTCATGCTATTGAGGGGTATATTACCCGTGGCGCGTGGGCGC
TAACCGATGCACTGCACATTAAAGCGATTGAAATCATTGCTGGGGCGCT
GCGAGGATCGGTTGCTGGTGATAAGGATGCCGGAGAAGAAATGGCGCTC
GGGCAGTATGTTGCGGGTATGGGCTTCTCGAATGTTGGGTTAGGGTTGG
TGCATGGTATGGCGCATCCACTGGGCGCGTTTTATAACACTCCACACGG
TGTTGCGAACGCCATCCTGTTACCGCATGTCATGCGTTATAACGCTGAC
TTTACCGGTGAGAAGTACCGCGATATCGCGCGCGTTATGGGCGTGAAAG
TGGAAGGTATGAGCCTGGAAGAGGCGCGTAATGCCGCTGTTGAAGCGGT
GTTTGCTCTCAACCGTGATGTCGGTATTCCGCCACATTTGCGTGATGTT
GGTGTACGCAAGGAAGACATTCCGGCACTGGCGCAGGCGGCACTGGATG
ATGTTTGTACCGGTGGCAACCCGCGTGAAGCAACGCTTGAGGATATTGT
AGAGCTTTACCATACCGCCTGGTAA

Gene ID 004 Amino Acid Sequence: Escherichia coli 1,2-propanediol oxidoreductase (resistant to oxidative stress) FucO_{I6L-L7V}

(SEQ ID NO: 26)
MMANRMLVNETAWFGRGAVGALTDEVKRRGYQKALIVTDKTLVQCGVVA
KVTDKMDAAGLAWAIYDGVVPNPTITVVKEGLGVFQNSGADYLIAIGGG
SPQDTCKAIGIISNNPEFADVRSLEGLSPTNKPSVPILAIPTTAGTAAE
VTINYVITDEEKRRKFVCVDPHDIPQVAFIDADMMDGMPPALKAATGVD
ALTHAIEGYITRGAWALTDALHIKAIEIIAGALRGSVAGDKDAGEEMAL
GQYVAGMGFSNVGLGLVHGMAHPLGAFYNTPHGVANAILLPHVMRYNAD
FTGEKYRDIARVMGVKVEGMSLEEARNAAVEAVFALNRDVGIPPHLRDV
GVRKEDIPALAQAALDDVCTGGNPREATLEDIVELYHTAW

Gene ID 005 Nucleotide Sequence: Ralstonia sp. S-6 Polyhydroxyalkanoate synthase phaC183*

(SEQ ID NO: 27)
ATGGCGACCGGCAAGGGCGCAGCAGCATCGACGCAGGAGGGCAAGAGCCA
ACCGTTTAAGGTGACTCCGGGTCCGTTTGACCCGGCGACGTGGCTGGAAT
GGAGCCGCCAATGGCAGGGTACCGAAGGCAATGGCCACGCAGCGGCCAGC
GGCATTCCGGGTCTGGATGCCCTGGCTGGCGTGAAGATTGCACCGGCGCA
ATTGGGCGACATTCAACAGCGCTATATGAAAGACTTCAGCGCCCTGTGGC
AAGCGATGGCGGAGGGCAAAGCGGAGGCAACCGGTCCGCTGCACGATCGT
CGCTTCGCGGGTGACGCGTGGCGTACGAACCTGCCGTACCGCTTTGCAGC
CGCATTTTACCTGTTGAATGCCCGTGCCTTGACCGAACTGGCGGACGCGG
TCGAGGCAGATGCGAAAACCCGTCAACGTATTCGTTTCGCGATCAGCCAA
TGGGTTGACGCAATGAGCCCAGCAAACTTCCTGGCGACGAACCCGGAGGC
GCAGCGCCGTCTGATCGAAAGCAACGGCGAGAGCCTGCGTGCTGGTCTGC
GCAACATGCTGGAGGACCTGACCCGTGGTAAAATCTCCCAAACCGATGAA
AGCGCCTTCGAAGTTGGTCGCAACGTCGCGGTCACCGAGGGTGCTGTGGT
TTACGAAAATGAGTATTTTCAGCTGCTGCAGTACAAGCCGTTGACCGCGA
AAGTGCACGCGCGTCCGCTGCTGATGGTGCCGCCGTGCATCAATAAGTAT
TACATCCTGGATCTGCAGCCGGAATCCAGCCTGGTCCGCCATATCGTTGA
GCAGGGCCATACGGTTTTCCTGGTGAGCTGGCGTAACCCGGATGCGAGCA
TGGCAGCGCGTACCTGGGATGACTATATCGAGCATGGCGCCATTCGTGCC
ATTGAAGTGGCGCGTGCTATCAGCGGTCAGCCGCGCATTAATGTCCTGGG
TTTTTGCGTGGGCGGTACCATTGTCTCCACTGCGCTGGCAGTTATGGCCG
GTCGTGGCGAACGTCCAGCCCAGAGCCTGACGCTGCTGACCACGCTGTTG
GATTTCTCCGATACTGGTGTGTTGGACGTTTTTGTCGACGAAGCACATGT
TCAGTTGCGTGAGGCGACCCTGGGCGGTGCTGCAGGTGCGCCGTGTGCGC
TGCTGCGTGGTATCGAGTTGGCGAATACCTTTAGCTTCCTGCGCCCGAAC
GATCTGGTTTGGAATTATGTGGTTGACAATTACCTGAAGGGCAACACCCC
GGTGCCATTTGATCTGTTGTTCTGGAACGGTGACGCGACCAACCTGCCGG
GTCCGTGGTATTGTTGGTATCTGCGCCATACGTACCTGCAAGACGAGCTG
AAGGTTCCGGGTAAGCTGACCGTTTGCGGCGTACCTGTGGACCTGGGTAA
AATCGACGTCCCGACGTACCTGTATGGTAGCCGTGAGGATCACATCGTCC
CGTGGACCGCGGCTTACGCGTCTACGCGTTTGCTGAGCAACGATCTGCGT
TTCGTCCTGGGTGCATCTGGTCACATCGCCGGTGTGATTAATCCACCAGC
CAAAAACAAACGCAGCCACTGGACGAATGATGCGCTGCCGGAAAGCCCGC
AGCAGTGGCTGGCAGGTGCGATTGAGCACCACGGCTCTTGGTGGCCGGAC
TGGACCGCATGGCTGGCCGGTCAAGCTGGTGCGAAACGTGCGGCTCCGGC
CAATTACGGCAATGCGCGTTACCGCGCTATTGAACCGGCACCTGGTCGTT
ACGTTAAAGCAAAGGCGTAA

Gene ID 005 Amino Acid Sequence: Ralstonia sp. S-6 Polyhydroxyalkanoate synthase PhaC183*

(SEQ ID NO: 28)
MATGKGAAASTQEGKSQPFKVTPGPFDPATWLEWSRQWQGTEGNGHAAAS
GIPGLDALAGVKIAPAQLGDIQQRYMKDFSALWQAMAEGKAEATGPLHDR
RFAGDAWRTNLPYRFAAAFYLLNARALTELADAVEADAKTRQRIRFAISQ
WVDAMSPANFLATNPEAQRRLIESNGESLRAGLRNMLEDLTRGKISQTDE
SAFEVGRNVAVTEGAVVYENEYFQLLQYKPLTAKVHARPLLMVPPCINKY
YILDLQPESSLVRHIVEQGHTVFLVSWRNPDASMAARTWDDYIEHGAIRA
IEVARAISGQPRINVLGFCVGGTIVSTALAVMAGRGERPAQSLTLLTTLL
DFSDTGVLDVFVDEAHVQLREATLGGAAGAPCALLRGIELANTFSFLRPN
DLVWNYVVDNYLKGNTPVPFDLLFWNGDATNLPGPWYCWYLRHTYLQDEL
KVPGKLTVCGVPVDLGKIDVPTYLYGSREDHIVPWTAAYASTRLLSNDLR
FVLGASGHIAGVINPPAKNKRSHWTNDALPESPQQWLAGAIEHHGSWWPD
WTAWLAGQAGAKRAAPANYGNARYRAIEPAPGRYVKAKA

Gene ID 006 Nucleotide Sequence: Trypanosoma brucei fumarate reductase (NADH-dependent) frd_g*

(SEQ ID NO: 29)
ATGGTAGACGGCCGCAGCAGCGCATCCATCGTCGCAGTCGACCCGGAGCG
TGCCGCACGCGAACGCGATGCGGCTGCGCGTGCCCTGTTGCAGGACAGCC
CGCTGCACACGACCATGCAGTATGCGACCTCGGGTCTGGAGCTGACTGTG
CCGTATGCACTGAAAGTTGTGGCAAGCGCTGATACCTTTGATCGTGCAAA
GGAAGTGGCGGACGAAGTCCTGCGCTGCGCATGGCAATTGGCAGATACCG
TTCTGAACAGCTTTAACCCTAACAGCGAGGTGAGCCTGGTCGGTCGCCTG
CCGGTTGGTCAAAAACATCAGATGTCCGCACCGCTGAAACGTGTCATGGC
GTGTTGCCAGCGCGTGTACAACTCCAGCGCCGGTTGCTTCGACCCGAGCA
CGGCGCCAGTCGCAAAAGCCTTGCGCGAAATTGCACTGGGTAAGGAGCGC
AATAACGCTTGCCTGGAGGCGCTGACCCAGGCTTGTACCCTGCCGAACAG
CTTCGTTATCGATTTCGAAGCGGGCACCATCAGCCGCAAACACGAACATG
CAAGCCTGGACCTGGGTGGCGTTTCGAAAGGCTATATCGTGGATTATGTG
ATTGACAACATCAATGCCGCTGGTTTCCAGAATGTTTTCTTCGATTGGGG
TGGTGACTGTCGTGCCTCCGGTATGAATGCGCGCAATACGCCGTGGGTCG
TCGGTATTACTCGCCCACCGAGCTTGGATATGCTGCCGAACCCGCCAAAG
GAAGCGAGCTATATCAGCGTCATCTCCCTGGACAACGAGGCGTTGGCGAC
CAGCGGTGATTACGAGAACCTGATCTACACCGCAGACGATAAGCCGTTGA
CCTGCACTTACGATTGGAAAGGTAAAGAGCTGATGAAGCCGAGCCAGAGC
AATATCGCTCAAGTTAGCGTGAAATGCTACAGCGCAATGTACGCCGATGC
CCTGGCAACGGCGTGCTTTATCAAGCGTGACCCGGCGAAAGTTCGTCAAC
TGCTGGACGGTTGGCGTTATGTTCGCGACACGGTCCGTGATTACCGTGTG
TACGTGCGTGAGAATGAGCGTGTAGCTAAGATGTTCGAGATTGCGACTGA
AGATGCGGAGATGCGTAAGCGTCGTATTAGCAATACTCTGCCTGCACGTG
TGATCGTGGTTGGTGGCGGTCTGGCGGGTCTGAGCGCTGCGATCGAAGCT
GCGGGCTGTGGTGCGCAGGTGGTCCTGATGGAGAAGGAAGCCAAGCTGGG
CGGTAACAGCGCGAAAGCTACCAGCGGTATCAACGGCTGGGGCACCCGTG
CGCAGGCTAAAGCGAGCATTGTTGATGGCGGCAAGTACTTTGAACGTGAC
ACTTACAAATCGGGTATTGGCGGTAATACTGATCCGGCACTGGTCAAAAC
CCTGTCCATGAAGAGCGCGGACGCGATTGGTTGGCTGACCAGCCTGGGCG
TCCCGCTGACCGTCCTGAGCCAGCTGGGTGGCCATAGCCGCAAGCGCACC
CATCGTGCACCGGACAAGAAAGACGGCACGCCTCTGCCAATCGGCTTTAC
CATCATGAAAACTCTGGAGGATCACGTCCGTGGTAATCTGTCTGGCCGTA
TCACCATCATGGAGAATTGTAGCGTTACCAGCCTGCTGAGCGAAACCAAG
GAACGCCCGGACGGCACGAAGCAGATCCGTGTGACGGGTGTCGAGTTTAC
CCAAGCGGGCTCTGGCAAGACCACCATCTTGGCGGATGCGGTTATCCTGG
CCACGGGTGGTTTCAGCAATGACAAGACGGCTGATAGCCTGCTGCGCGAA
CACGCACCGCACCTGGTTAACTTTCCGACCACCAACGGCCCGTGGGCGAC
GGGTGATGGTGTGAAGTTGGCTCAGCGTCTGGGTGCTCAACTGGTCGATA
TGGATAAAGTTCAGCTGCACCCGACCGGCCTGATTAATCCGAAAGACCCG
GCCAATCCGACCAAATTCCTGGGTCCTGAAGCGTTGCGTGGTAGCGGTGG
TGTGCTGCTGAATAAACAAGGTAAACGTTTTGTGAATGAGCTGGATCTGC
GTAGCGTGGTTAGCAAAGCCATTATGGAGCAAGGTGCCGAGTATCCGGGC
AGCGGTGGCAGCATGTTCGCGTATTGTGTTCTGAACGCTGCGGCACAAAA
ACTGTTCGGCGTTTCTTCGCATGAGTTTTACTGGAAAAAGATGGGCTTGT
TCGTGAAGGCCGATACCATGCGCGACCTGGCGGCTCTGATCGGTTGTCCG
GTTGAGAGCGTCCAACAAACGCTGGAAGAGTATGAACGTCTGAGCATTAG
CCAACGCAGCTGCCCGATCACCCGTAAGTCTGTGTACCCGTGTGTTCTGG
GTACGAAAGGCCCGTACTATGTGGCGTTCGTGACCCCGAGCATTCACTAT
ACGATGGGCGGTTGTTTGATCAGCCCGAGCGCGGAGATCCAAATGAAGAA
CACCAGCTCTCGTGCGCCGCTGTCCCATAGCAACCCGATCCTGGGTCTGT
TTGGCGCAGGCGAAGTGACCGGCGGTGTGCACGGTGGTAACCGCCTGGGC
GGCAACAGCTTGCTGGAGTGCGTCGTCTTTGGTCGTATTGCAGGTGACCG
TGCGAGCACCATTCTGCAACGCAAGTCTAGCGCACTGTCCTTTAAAGTTT
GGACCACCGTCGTTCTGCGTGAGGTTCGCGAGGGTGGTGTCTATGGTGCG
GGCAGCCGTGTGCTGCGTTTTAACCTGCCAGGCGCGCTGCAACGCTCTGG
TCTGTCCCTGGGCCAGTTCATCGCGATTCGTGGTGATTGGGACGGTCAAC
AGTTGATTGGCTATTACTCCCCGATTACCCTGCCTGACGACCTGGGTATG
ATTGACATTCTGGCACGCAGCGACAAGGGTACGCTGCGTGAGTGGATTAG
CGCGCTGGAACCGGGTGACGCGGTGGAGATGAAAGCGTGTGGTGGCCTGG
TGATTGAGCGTCGTCTGAGCGATAAGCACTTCGTGTTTATGGGCCACATC
ATCAATAAACTGTGCTTGATTGCCGGTGGTACGGGTGTTGCACCGATGCT
GCAAATCATCAAAGCGGCATTCATGAAGCCGTTTATCGATACGTTGGAAA
GCGTTCATCTGATCTATGCGGCCGAGGATGTTACTGAATTGACCTACCGC
GAAGTTTTGGAGGAGCGTCGCCGTGAAAGCCGTGGTAAATTCAAAAAGAC
GTTCGTGTTGAACCGTCCTCCGCCGCTGTGGACGGATGGTGTCGGCTTTA
TTGACCGTGGCATTCTGACCAATCATGTTCAGCCGCCGTCCGACAATCTG
CTGGTGGCCATTTGTGGTCCGCCTGTGATGCAACGCATTGTTAAAGCGAC
CCTGAAAACCCTGGGTTACAATATGAATCTGGTTCGTACCGTGGACGAAA
CGGAACCGAGCGGTAGCTAA

Gene ID 006 Amino Acid Sequence: Trypanosoma brucei fumarate reductase (NADH-dependent) Frd_g*

(SEQ ID NO: 30)
MVDGRSSASIVAVDPERAARERDAAARALLQDSPLHTTMQYATSGLEL
TVPYALKVVASADTFDRAKEVADEVLRCAWQLADTVLNSFNPNSEVSL
VGRLPVGQKHQMSAPLKRVMACCQRVYNSSAGCFDPSTAPVAKALREI
ALGKERNNACLEALTQACTLPNSFVIDFEAGTISRKHEHASLDLGGVS
KGYIVDYVIDNINAAGFQNVFFDWGGDCRASGMNARNTPWVVGITRPP
SLDMLPNPPKEASYISVISLDNEALATSGDYENLIYTADDKPLTCTYD
WKGKELMKPSQSNIAQVSVKCYSAMYADALATACFIKRDPAKVRQLLD
GWRYVRDTVRDYRVYVRENERVAKMFEIATEDAEMRKRRISNTLPARV
IVVGGGLAGLSAAIEAAGCGAQVVLMEKEAKLGGNSAKATSGINGWGT
RAQAKASIVDGGKYFERDTYKSGIGGNTDPALVKTLSMKSADAIGWLT
SLGVPLTVLSQLGGHSRKRTHRAPDKKDGTPLPIGFTIMKTLEDHVRG
NLSGRITIMENCSVTSLLSETKERPDGTKQIRVTGVEFTQAGSGKTTI
LADAVILATGGFSNDKTADSLLREHAPHLVNFPTTNGPWATGDGVKLA
QRLGAQLVDMDKVQLHPTGLINPKDPANPTKFLGPEALRGSGGVLLNK
QGKRFVNELDLRSVVSKAIMEQGAEYPGSGGSMFAYCVLNAAAQKLFG
VSSHEFYWKKMGLFVKADTMRDLAALIGCPVESVQQTLEEYERLSISQ
RSCPITRKSVYPCVLGTKGPYYVAFVTPSIHYTMGGCLISPSAEIQMK
NTSSRAPLSHSNPILGLFGAGEVTGGVHGGNRLGGNSLLECVVFGRIA
GDRASTILQRKSSALSFKVWTTVVLREVREGGVYGAGSRVLRFNLPGA
LQRSGLSLGQFIAIRGDWDGQQLIGYYSPITLPDDLGMIDILARSDKG
TLREWISALEPGDAVEMKACGGLVIERRLSDKHFVFMGHIINKLCLIA
GGTGVAPMLQIIKAAFMKPFIDTLESVHLIYAAEDVTELTYREVLEER
RRESRGKFKKTFVLNRPPPLWTDGVGFIDRGILTNHVQPPSDNLLVAI
CGPPVMQRIVKATLKTLGYNMNLVRTVDETEPSGS

Gene ID 002 Nucleotide Sequence: Clostridium kluyveri succinate semialdehyde dehydrogenase sucD*

(SEQ ID NO. 31)
ATGTCCAACGAGGTTAGCATTAAGGAGCTGATTGAGAAGGCGAAAGTGGC
GCAGAAAAAGCTGGAAGCGTATAGCCAAGAGCAAGTTGACGTTCTGGTCA
AGGCGCTGGGTAAAGTTGTGTACGACAACGCCGAGATGTTCGCGAAAGAG
GCGGTGGAGGAAACCGAGATGGGTGTTTACGAGGATAAAGTGGCTAAATG
TCATCTGAAATCTGGTGCAATCTGGAATCACATTAAAGATAAGAAAACCG
TTGGTATTATCAAGGAAGAACCGGAGCGTGCGCTGGTGTACGTCGCGAAG
CCTAAAGGTGTTGTGGCGGCGACGACCCCTATCACCAATCCTGTGGTTAC
CCCGATGTGTAACGCGATGGCAGCAATTAAAGGTCGCAACACCATCATTG
TCGCCCCGCATCCGAAGGCGAAGAAGGTGAGCGCGCACACCGTGGAGCTG
ATGAATGCAGAACTGAAAAAGTTGGGTGCGCCGGAAAACATTATCCAGAT
CGTTGAAGCCCCAAGCCGTGAAGCAGCCAAGGAGTTGATGGAGAGCGCAG
ACGTGGTTATCGCCACGGGTGGCGCAGGCCGTGTTAAAGCAGCGTACTCC
TCCGGCCGTCCGGCATACGGTGTCGGTCCGGGCAATTCTCAGGTCATTGT
CGATAAGGGTTACGATTATAACAAAGCTGCCCAGGACATCATTACCGGCC
GCAAGTATGACAACGGTATCATTTGCAGCTCTGAGCAGAGCGTGATCGCA
CCGGCGGAGGACTACGACAAGGTCATCGCGGCTTTCGTCGAGAATGGCGC
GTTCTATGTCGAGGATGAGGAAACTGTGGAGAAATTCCGTAGCACGCTGT
TCAAGGATGGCAAGATCAATAGCAAAATCATCGGTAAATCCGTGCAGATC
ATCGCTGACCTGGCTGGTGTCAAGGTGCCGGAAGGCACCAAGGTGATCGT
GTTGAAGGGCAAGGGTGCCGGTGAAAAGGACGTTCTGTGCAAGGAGAAAA
TGTGCCCGGTCCTGGTTGCCCTGAAATATGACACCTTTGAGGAGGCGGTC
GAGATCGCGATGGCCAACTATATGTACGAGGGTGCGGGCCATACCGCCGG
TATCCACAGCGATAACGACGAGAATATCCGCTACGCGGGTACGGTGCTGC
CAATCAGCCGTCTGGTTGTCAACCAGCCAGCAACTACGGCCGGTGGTAGC
TTTAACAATGGTTTTAATCCGACCACCACCTTGGGCTGCGGTAGCTGGGG
CCGTAACTCCATTAGCGAGAACCTGACGTATGAGCATCTGATTAATGTCA
GCCGTATTGGCTATTTCAATAAGGAGGCAAAAGTTCCTAGCTACGAGGAG
ATCTGGGGTTAA

Gene ID 002 Protein Sequence: Clostridium kluyveri succinate semialdehyde dehydrogenase sucD*

(SEQ ID NO. 32)
MSNEVSIKELIEKAKVAQKKLEAYSQEQVDVLVKALGKVVYDNAEMFAK
EAVEETEMGVYEDKVAKCHLKSGAIWNHIKDKKTVGIIKEEPERALVYV
AKPKGVVAATTPITNPVVTPMCNAMAAIKGRNTIIVAPHPKAKKVSAHT
VELMNAELKKLGAPENIIQIVEAPSREAAKELMESADVVIATGGAGRVK
AAYSSGRPAYGVGPGNSQVIVDKGYDYNKAAQDIITGRKYDNGIICSSE
QSVIAPAEDYDKVIAAFVENGAFYVEDEETVEKFRSTLFKDGKINSKII
GKSVQIIADLAGVKVPEGTKVIVLKGKGAGEKDVLCKEKMCPVLVALKY
DTFEEAVEIAMANYMYEGAGHTAGIHSDNDENIRYAGTVLPISRLVVNQ
PATTAGGSFNNGFNPTTTLGCGSWGRNSISENLTYEHLINVSRIGYFNK
EAKVPSYEEIWG

Gene ID 003 Nucleotide Sequence: Arabidopsis thaliana succinic semialdehyde reductase ssaR_{At}*

(SEQ ID NO. 33)
ATGGAAGTAGGTTTTCTGGGTCTGGGCATTATGGGTAAAGCTATGTCCA
TGAACCTGCTGAAAAACGGTTTCAAAGTTACCGTGTGGAACCGCACTCT
GTCTAAATGTGATGAACTGGTTGAACACGGTGCAAGCGTGTGCGAGTCT
CCGGCTGAGGTGATCAAGAAATGCAAATACACGATCGCGATGCTGAGCG
ATCCGTGTGCAGCTCTGTCTGTTGTTTTCGATAAAGGCGGTGTTCTGGA
ACAGATCTGCGAGGGTAAGGGCTACATCGACATGTCTACCGTCGACGCG
GAAACTAGCCTGAAAATTAACGAAGCGATCACGGGCAAAGGTGGCCGTT
TTGTAGAAGGTCCTGTTAGCGGTTCCAAAAAGCCGGCAGAAGACGGCCA
GCTGATCATCCTGGCAGCAGGCGACAAAGCACTGTTCGAGGAATCCATC
CCGGCCTTTGATGTACTGGGCAAACGTTCCTTTTATCTGGGTCAGGTGG
GTAACGGTGCGAAAATGAAACTGATTGTTAACATGATCATGGGTTCTAT
GATGAACGCGTTTAGCGAAGGTCTGGTACTGGCAGATAAAAGCGGTCTG
TCTAGCGACACGCTGCTGGATATTCTGGATCTGGGTGCTATGACGAATC
CGATGTTCAAAGGCAAAGGTCCGTCCATGACTAAATCCAGCTACCCACC
GGCTTTCCCGCTGAAACACCAGCAGAAAGACATGCGTCTGGCTCTGGCT
CTGGGCGACGAAAACGCTGTTAGCATGCCGGTCGCTGCGGCTGCGAACG
AAGCCTTCAAGAAAGCCCGTAGCCTGGGCCTGGGCGATCTGGACTTTTC
TGCTGTTATCGAAGCGGTAAAATTCTCTCGTGAATAA

Gene ID 003 Protein Sequence: Arabidopsis thaliana succinic semialdehyde reductase ssaR_{At}*

(SEQ ID NO. 34)
MEVGFLGLGIMGKAMSMNLLKNGFKVTVWNRTLSKCDELVEHGASVCES
PAEVIKKCKYTIAMLSDPCAALSVVFDKGGVLEQICEGKGYIDMSTVDA
ETSLKINEAITGKGGRFVEGPVSGSKKPAEDGQLIILAAGDKALFEESI
PAFDVLGKRSFYLGQVGNGAKMKLIVNMIMGSMMNAFSEGLVLADKSGL
SSDTLLDILDLGAMTNPMFKGKGPSMTKSSYPPAFPLKHQQKDMRLALA
LGDENAVSMPVAAAANEAFKKARSLGLGDLDFSAVIEAVKFSRE

Gene ID 006 Nucleotide Sequence: Pseudomonas putida/Ralstonia eutropha JMP134 Polyhydroxyalkanoate synthase fusion protein phaC3/C1

(SEQ ID NO. 35)
ATGACTAGAAGGAGGTTTCATATGAGTAACAAGAACAACGATGAGCTGG
CGACGGGTAAAGGTGCTGCTGCATCTTCTACTGAAGGTAAATCTCAGCC
GTTTAAATTCCCACCGGGTCCGCTGGACCCGGCCACTTGGCTGGAATGG
AGCCGTCAGTGGCAAGGTCCGGAGGGCAATGGCGGTACCGTGCCGGGTG
GCTTTCCGGGTTTCGAAGCGTTCGCGGCGTCCCCGCTGGCGGGCGTGAA
AATCGACCCGGCTCAGCTGGCAGAGATCCAGCAGCGTTATATGCGTGAT
TTCACCGAGCTGTGGCGTGGTCTGGCAGGCGGTGACACCGAGAGCGCTG
GCAAACTGCATGACCGTCGCTTCGCGTCCGAAGCGTGGCACAAAAACGC
GCCGTATCGCTATACTGCGGCATTTTACCTGCTGAACGCACGTGCACTG
ACGGAACTGGCTGATGCAGTAGAAGCGGATCCGAAAACCCGTCAGCGTA
TCCGTTTTGCGGTTTCCCAGTGGGTAGATGCTATGAGCCCGGCTAACTT
CCTGGCCACCAACCCGGACGCTCAGAACCGTCTGATCGAGAGCCGTGGT
GAAAGCCTGCGTGCCGGCATGCGCAATATGCTGGAAGATCTGACCCGCG
GTAAAATTTCCCAAACCGATGAGACTGCCTTCGAAGTAGGCCGTAACAT
GGCAGTTACCGAAGGTGCTGTGGTATTCGAAAACGAGTTCTTCCAGCTG
CTGCAGTACAAACCTCTGACTGACAAAGTATACACCCGTCCGCTGCTGC
TGGTACCGCCGTGCATTAACAAGTTCTATATTCTGGACCTGCAGCCGGA
AGGTTCTCTGGTCCGTTACGCAGTCGAACAGGGTCACACTGTATTCCTG
GTGAGCTGGCGCAATCCAGACGCTAGCATGGCTGGCTGTACCTGGGATG
ACTATATTGAAAACGCGGCTATCCGCGCCATCGAGGTTGTGCGTGATAT
CAGCGGTCAGGACAAGATCAACACCCTGGGCTTTTGTGTTGGTGGCACG
ATCATCTCCACTGCCCTGGCGGTCCTGGCCGCCCGTGGTGAGCACCCGG
TGGCCTCTCTGACCCTGCTGACTACCCTGCTGGACTTCACCGATACTGG
TATCCTGGATGTTTTCGTGGACGAGCCACACGTTCAGCTGCGTGAGGCG
ACTCTGGGCGGCGCCAGCGGCGGTCTGCTGCGTGGTGTCGAGCTGGCCA
ATACCTTTTCCTTCCTGCGCCCGAACGACCTGGTTTGGAACTACGTTGT
TGACAACTATCTGAAAGGCAACACCCCGGTACCTTTCGATCTGCTGTTC
TGGAACGGTGATGCAACCAACCTGCCTGGTCCATGGTACTGTTGGTACC
TGCGTCATACTTACCTGCAGAACGAACTGAAAGAGCCGGGCAAACTGAC
CGTGTGTAACGAACCTGTGGACCTGGGCGCGATTAACGTTCCTACTTAC
ATCTACGGTTCCCGTGAAGATCACATCGTACCGTGGACCGCGGCTTACG
CCAGCACCGCGCTGCTGAAGAACGATCTGCGTTTCGTACTGGGCGCATC
CGGCCATATCGCAGGTGTGATCAACCCTCCTGCAAAGAAAAAGCGTTCT
CATTGGACCAACGACGCGCTGCCAGAATCCGCGCAGGATTGGCTGGCAG
GTGCTGAGGAACACCATGGTTCCTGGTGGCCGGATTGGATGACCTGGCT
GGGTAAACAAGCCGGTGCAAAACGTGCAGCTCCAACTGAATATGGTAGC
AAGCGTTATGCTGCAATCGAGCCAGCGCCAGGCCGTTACGTTAAAGCGA
AAGCATAA

Gene ID 006 Protein Sequence: Pseudomonas putida/Ralstonia eutropha JMP134 Polyhydroxyalkanoate synthase fusion protein phaC3/C1

(SEQ ID NO. 36)
MSNKNNDELATGKGAAASSTEGKSQPFKFPPGPLDPATWLEWSRQWQGP
EGNGGTVPGGFPGFEAFAASPLAGVKIDPAQLAEIQQRYMRDFTELWRG
LAGGDTESAGKLHDRRFASEAWHKNAPYRYTAAFYLLNARALTELADAV
EADPKTRQRIRFAVSQWVDAMSPANFLATNPDAQNRLIESRGESLRAGM
RNMLEDLTRGKISQTDETAFEVGRNMAVTEGAVVFENEFFQLLQYKPLT
DKVYTRPLLLVPPCINKFYILDLQPEGSLVRYAVEQGHTVFLVSWRNPD
ASMAGCTWDDYIENAAIRAIEVVRDISGQDKINTLGFCVGGTIISTALA
VLAARGEHPVASLTLLTTLLDFTDTGILDVFVDEPHVQLREATLGGASG
GLLRGVELANTFSFLRPNDLVWNYVVDNYLKGNTPVPFDLLFWNGDATN
LPGPWYCWYLRHTYLQNELKEPGKLTVCNEPVDLGAINVPTYIYGSRED
HIVPWTAAYASTALLKNDLRFVLGASGHIAGVINPPAKKKRSHWTNDAL
PESAQDWLAGAEEHHGSWWPDWMTWLGKQAGAKRAAPTEYGSKRYAAIE
PAPGRYVKAKA

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method of increasing the production of a 4-carbon (C4) product or a polymer of 4-carbon monomers from a renewable feedstock, comprising
a) providing a genetically modified organism having a modified metabolic C4 pathway, and
b) providing one or more genes that are stably expressed that encodes one or more enzymes having an activity of i) catalyzing the decarboxylation of alpha-ketoglutarate to succinic semialdehyde; ii) catalyzing the conversion of malonyl CoA to malonate semialdehyde iii) catalyzing the conversion of L-lactaldehyde to L-1,2-propanediol and having increased resistance to oxidative stress; iv) catalyzing fumarate to succinate; v) catalyzing the carboxylation of pyruvate; or vi) catalyzing NADH to NADPH; wherein the production of the product or polymer is improved compared to a wild type or the modified organism of step a) and/or the carbon flux from the renewable feedstock 4-carbon (C4) product or a polymer of 4-carbon monomers is increased.

2. The method of claim 1, wherein the 4-carbon product is selected from: gamma butyrolactone, 1,4-butanediol, tetrahydrofuran, N-methylpyrrolidone, N-ethylpyrrolidone, 2-pyrrolidinone, N-vinylpyrrolidone, polyvinylpyrrolidone, succinic acid, 1,4-butanediamide, succinonitrile, succinamide and 2-pyrrolidone (2-Py).

3. The method of claim 1, wherein the organism having a modified metabolic C4 pathway has a modified poly-4-hydroxybutyrate pathway and the production of poly-4-hydroxybutyrate is increased.

4. The method of claim 1, wherein the one or more genes that are stably expressed encode one or more enzymes selected from: an alpha-ketoglutarate decarboxylase, an 2-oxoglutarate decarboxylase, a malonyl-CoA reductase, an NADH-dependent fumarate reductase, an oxidative stress-resistant 1,2 propanediol oxidoreducatase, a pyruvate carboxylase and an NADH kinase.

5. A method of increasing the production of 4-hydroxybutyrate or poly-4-hydroxybutyrate, comprising
a) providing a genetically modified organism having a modified metabolic 4-hdyroxybutyrate pathway, and
b) providing one or more genes that are stably expressed that encodes one or more enzymes selected from: an alpha-ketoglutarate decarboxylase or a 2-oxoglutarate decarboxylase enzyme, a malonyl-CoA reductase having activity for converting to Suc-CoA to succinic semialdehyde, an oxidative stress-resistant 1,2 propanediol oxidoreducatase having activity for converting SSA to 4-hydroxybutyrate; a NADH-dependent fumarate reductase having activity for converting fumarate to succinate, a pyruvate carboxylase having activity of converting pyruvate to form oxaloacetate and an NADH kinase wherein intracellular NADPH concentrations are increased, wherein the expression increases the production of 4-hydroxybutyrate or poly-4-hydroxybutyrate.

6. The method of claim 1 wherein the one or more enzyme is selected from an alpha-ketoglutarate decarboxylase from Pseudonocardia dioxanivorans or mutants and homologues thereof; an 2-oxoglutaratedecarboxylase enzyme from Synechococcus sp. PCC 7002 or mutants and homologues thereof; a malonyl-CoA reductase from Metallosphaera sedula or mutants and homologues thereof; a malonyl-CoA reductase from Sulfolous tokodaii or mutants and homologues thereof; an oxidative stress-resistant 1,2 propanediol oxidoreducatase from E. coli, mutants and homologues thereof; an NADH-dependent fumarate reductase from Trypanosoma brucei, mutants and homologues thereof; a pyruvate carboxylase from L. lactis, mutants and homologues thereof and an NADH kinase from Aspergillus nidulans, mutants and homologues thereof.

7. A method of increasing the production of a 4-carbon (C4) product or a polymer of 4-carbon monomers from a renewable feedstock, comprising
providing a genetically modified organism having a reduced activity of alpha-ketoglutarate dehydrogenase such that growth is impaired as compared to a wild-type organism without the reduced activity; and
providing one or more genes that are stably expressed that encodes one or more enzymes having an activity of catalyzing the decarboxylation of alpha-ketoglutarate to succinic semialdehyde wherein growth is improved and the carbon flux from the renewable feedstock 4-carbon (C4) product or a polymer of 4-carbon monomers is increased.

8. A method of producing an increase of poly-4-hydroxybutyrate in a genetically modified organism (recombinant host) having a poly-4-hydroxybutyrate pathway, comprising stably expressing from the host organism a gene encoding an alpha-ketoglutarate decarboxylase or a 2-oxoglutarate decarboxylase enzyme, wherein the alpha-ketoglutarate decarboxylase or 2-oxoglutaratedecarboxylase catalyzes the decarboxylation of alpha-ketoglutarate to succinic semialdehyde and increases the amount of poly-4-hydroxybutyrate in the organism.

9. The method of claim 1 wherein the enzyme is alpha-ketoglutarate decarboxylase from Pseudonocardia dioxanivorans or mutants and homologues thereof or the 2-oxoglutaratedecarboxylase enzyme is from Synechococcus sp. PCC 7002 or mutants and homologues thereof.

10. The method of claim 9, wherein the alpha-ketoglutarate decarboxylase from P. dioxanivorans comprises a mutation of an alanine to threonine at amino acid position 887.

11. The method of claim 1, wherein the organism further has a stably incorporated gene encoding a succinate semialdehyde dehydrogenase converts succinyl-CoA to succinic semialdehyde.

12. The method of claim 11, wherein the succinate semialdehyde dehydrogenase is from Clostridium kluyveri or homologues thereof.

13. The method of claim 3, wherein the genetically engineered organism having a poly-4-hydroxybutyrate pathway has an inhibiting mutation in its CoA-independent NAD-dependent succinic semialdehyde dehydrogenase gene or its CoA-independent NADP-dependent succinic semialdehyde dehydrogenase gene, or having inhibiting mutations in both genes, and having stably incorporated one or more genes encoding one or more enzymes selected from a succinate semialdehyde dehydrogenase wherein the succinate semialdehyde dehydrogenase converts succinyl-CoA to succinic semialdehyde, a succinic semialdehyde reductase wherein the succinic semialdehyde reductase converts succinic semialdehyde to 4-hydroxybutyrate, a CoA transferase wherein the CoA transferase converts 4-hydroxybutyrate to 4-hydroxybutyryl-CoA, and a polyhydroxyalkanoate synthase wherein the polyhydroxyalkanoate synthase polymerizes 4-hydroxybutyryl-CoA to poly-4-hydroxybutyrate.

14. The method of claim 13, wherein the organism has a disruption in one or more genes selected from yneI, gabD, pykF, pykA, astD and sucCD or a reduced activity in the gene product.

15. The method of claim 1, wherein the method further includes culturing a genetically engineered organism with a renewable feedstock to produce a biomass.

16. The method of claim 15, wherein a source of the renewable feedstock is selected from glucose, levoglucosan, fructose, sucrose, arabinose, maltose lactose xylose, fatty acids, vegetable oils, and biomass derived synthesis gas or a combination thereof.

17. The method of claim 15, wherein the culturing includes addition of pantothenate in a fermentation media, wherein an increase in growth or production occurs.

18. The method of claim 16, wherein the organism is a bacteria, yeast, fungi, algae, cyanobacteria, or a mixture of any two or more thereof.

19. The method of claim 18, wherein the organism is a bacteria.

20. The method of claim 19, wherein the bacteria is selected from Escherichia coli, Ralstonia eutropha, Zoogloea ramigera, Allochromatium vinosum, Rhodococcus ruber, Delftia acidovorans, Aeromonas caviae, Synechocystis sp. PCC 6803, Synechococcus elongatus PCC 7942, Thiocapsa pfenigii, Bacillus megaterium, Acinetobacter baumannii, Acinetobacter baylyi, Clostridium kluyveri, Methylobacterium extorquens, Nocardia corralina, Nocardia salmonicolor, Pseudomonas fluorescens, Pseudomonas oleovorans, Pseudomonas sp. 6-19, Pseudomonas sp. 61-3 and Pseudomonas putida, Rhodobacter sphaeroides, Alcaligenes latus, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Saccharomyces cerevisiae, Schizosaccharomyces pombe, Kluyveromyces lactis, Kluyveromyces marxianus, Aspergillus terreus, Aspergillus niger and Pichia pastoris.

21-32. (canceled)


SEQUENCE LISTING (36 sequences)

SEQ ID NO: 1 - DNA, 35 bp, Artificial Sequence
  synthetic oligonucleotide
ttgacagctagctcagtcctaggtataatgctagc

SEQ ID NO: 2 - DNA, 35 bp, Artificial Sequence
  synthetic oligonucleotide
ttgacagctagctcagtcctaggtactgtgctagc

SEQ ID NO: 3 - DNA, 35 bp, Artificial Sequence
  synthetic oligonucleotide
tttacagctagctcagtcctaggtattatgctagc

SEQ ID NO: 4 - DNA, 35 bp, Artificial Sequence
  synthetic oligonucleotide
ctgacagctagctcagtcctaggtataatgctagc

SEQ ID NO: 5 - DNA, 35 bp, Artificial Sequence
  synthetic oligonucleotide
tttacggctagctcagtcctaggtacaatgctagc

SEQ ID NO: 6 - DNA, 35 bp, Artificial Sequence
  synthetic oligonucleotide
ttgacagctagctcagtcctagggactatgctagc

SEQ ID NO: 7 - DNA, 30 bp, Artificial Sequence
  synthetic oligonucleotide
ttgacaattaatcatccggctcgtataatg

SEQ ID NO: 8 - DNA, 33 bp, Artificial Sequence
  synthetic oligonucleotide
ttgacaattaatcatcgtcgtataatgtgtgga

SEQ ID NO: 9 - DNA, 54 bp, Artificial Sequence
  synthetic oligonucleotide
tccctatcagtgatagagattgacatccctatcagtgatagagatactgagcac

SEQ ID NO: 10 - DNA, 31 bp, Artificial Sequence
  synthetic oligonucleotide
tcgccagtctggcctgaacatgatataaaat

SEQ ID NO: 11 - DNA, 178 bp, Artificial Sequence
  synthetic oligonucleotide
aaccactatcaatatattcatgtcgaaaatttgtttatctaacgagtaagcaaggcggattgacggatcatccgggtcgctataaggtaaggatggtcttaacactgaatccttacggctgggttagccccgcgcacgtagttcgcaggacgcgggtgacgtaacggcacaagaaacg

SEQ ID NO: 12 - DNA, 170 bp, Artificial Sequence
  synthetic oligonucleotide
atgcgggttgatgtaaaactttgttcgcccctggagaaagcctcgtgtatactcctcacccttataaaagtccctttcaaaaaaggccgcggtgctttacaaagcagcagcaattgcagtaaaattccgcaccattttgaaataagctggcgttgatgccagcggcaaac

SEQ ID NO: 13 - DNA, 35 bp, Artificial Sequence
  synthetic oligonucleotide
ttgacagctagctcagtcctaggtacagtgctagc

SEQ ID NO: 14 - DNA, 35 bp, Artificial Sequence
  synthetic oligonucleotide
ttgacagctagctcagtcctaggtacaatgctagc

SEQ ID NO: 15 - DNA, 29 bp, Artificial Sequence
  synthetic oligonucleotide
ctaatgagcgggcttttttttgaacaaaa

SEQ ID NO: 16 - DNA, 38 bp, Artificial Sequence
  synthetic oligonucleotide
aaaaaaaaaaaaccccgcttcggcggggtttttttttt

SEQ ID NO: 17 - DNA, 44 bp, Artificial Sequence
  synthetic oligonucleotide
ataaaacgaaaggctcagtcgaaagactgggcctttcgttttat

SEQ ID NO: 18 - DNA, 28 bp, Artificial Sequence
  synthetic oligonucleotide
agaaggccatcctgacggatggcctttt

SEQ ID NO: 19 - DNA, 3414 bp, Lactococcus lactis
atgaaaaaactactcgtcgccaatcgtggagaaatcgccgttcgtgtctttcgtgcctgtaatgaactcggactttctacagtagccgtctatgcaagagaagatgaatattccgttcatcgctttaaagcagatgaatcttaccttatcggtcaaggtaaaaaaccaattgatgcttatttggatattgatgatattattcgtgttgctcttgaatcaggagcagatgccattcatcccggttatggtcttttatctgaaaatcttgaatttgctacaaaagttcgagcagcaggattagtttttgtcggtcctgaacttcatcatttggatattttcggcgataaaatcaaagcaaaagccgcagctgatgaagctcaagttcccggaattcccggaacaaatggtgcagtagatattgacggagctcttgaatttgctcaaacttacggatatccagtcatgattaaggcagcattgggcggcggcggtcgtggaatgcgtgttgcgcgtaatgacgctgaaatgcacgacggatatgctcgtgcgaaatcagaagctatcggtgcctttggttctggagaaatctatgttgaaaaatacattgaaaatcctaagcatattgaagttcaaattcttggggatagtcatggaaatattgtccatttgcacgaacgtgattgctctgtccaacgccgaaatcaaaaagtcattgaaattgctccagccgtaggactctcaccagagttccgtaatgaaatttgtgaagcagcagttaaactttgtaaaaatgttggctatgttaatgctgggacggttgaatttttagtcaaagatgataagttctactttatcgaagtcaacccacgtgttcaagttgaacacacaattaccgagcttattacaggtgtagatattgttcaagcacaaattttgattgctcaaggcaaagatttacatacagaaattggtatcccggcacaagctgaaataccacttttgggctcagccattcaatgtcgtattactacagaagacccgcaaaatggcttcttgccagatacaggtaaaatcgatacctaccgttcaccaggtggtttcggcattcgtttggacgttggaaatgcctatgctggttatgaagtgactccctattttgactcgcttttagtaaaagtttgtacctttgctaatgaatttagcgatagtgtacgtaaaatggatcgtgtgcttcatgaattccgtattcgtggggtgaaaactaatattccatttttgattaatgttattgccaatgaaaactttacgagcggacaagcaacaacaacctttattgacaatactccaagtcttttcaatttcccacgcttacgtgaccgtggaacaaaaaccttacactacttatcaatgattacagtcaatggtttcccagggattgaaaatacagaaaaacgccattttgaagaacctcgtcaacctctacttaacattgaaaagaaaaagacagctaaaaatatcttagatgaacaaggggctgatgcggtagttgaatatgtgaaaaatacaaaagaagtattattgacagatacaactttacgtgatgctcaccagtctcttcttgccactcgtttgcgtttgcaagatatgaaaggaattgctcaagccattgaccaaggacttccagaacttttctcagctgaaatgtggggtggggcaacctttgatgtcgcttatcgtttcttgaatgaatcgccttggtatcgtctacgtaaattacgtaaactcatgccaaataccatgttccaaatgcttttccgtggttcaaatgcagttggatatcaaaactatcctgataatgtcattgaagaatttatccacgtagctgcacatgaaggaatcgatgtctttcgtatctttgatagcctcaactggttgccacaaatggaaaaatcaatccaagcagtgcgtgataatggaaaaattgccgaagcaaccatttgttatacaggagatatccttgacccaagtcgaccaaaatataatatccaatactacaaagatttggcaaaagagttagaagctactggggctcatatacttgccgttaaagatatggcgggcttgttgaaacctcaagcggcatatcgcttgatttcagaattaaaagatacggttgacttaccaattcacttgcatacacatgatacttcaggaaatggtattattacctattctggtgcaactcaagcaggagtagatattattgatgtggcaactgccagtcttgctggtggaacttctcaaccttcaatgcaatcaatttattatgcccttgaacatggtccccgtcatgcttcaattaatgtgaaaaatgcagagcaaattgaccattattgggaagatgtgcgtaaatattatgcaccttttgaggcaggaattacgagcccacaaactgaagtttacatgcatgaaatgcctggcggacaatatactaacttgaaatctcaagcagcagctgttggacttggacatcgttttgatgaaatcaaacaaatgtatcgtaaagtaaacatgatgtttggcgatatcattaaagtaactccttcatcaaaagtagttggtgatatggcactctttatgattcaaaacgaattgacagaagaggatgtctatgcgcgaggaaatgagcttaacttccctgaatcagtagtctcattcttccgtggtgatttaggacagcctgttggaggtttcccagaagaactacaaaaaattattgtaaaagacaaatcggtcattatggatcgtccaggattacatgccgaaaaagttgattttgcaactgtaaaagctgacttggaacaaaaaattggttatgaaccaggtgatcatgaagttatctcttacattatgtatccacaagttttccttgattatcaaaaaatgcaaagagaatttggagctgtcacactactcgatactccaactttcttacacggaatgcgcctcaatgaaaaaattgaagtccaaattgaaaaaggtaaaacgctcagcattcgtttagatgaaataggagaacctgacctcgctggaaatcgtgtgctcttctttaacttgaacggtcagcgtcgtgaagttgttattaatgaccaatccgttcaaactcaaattgtagctaaacgtaaggccgaaacaggtaatccaaaccaaattggagcaactatgcccggttctgttcttgaaatcctagttaaagctggagataaagttaaaaaaggacaagctttgatggttactgaagccatgaagatggaaacgaccattgagtcaccatttgatggagaggttattgcccttcatgttgtcaaaggtgaagccattcaaacacaagacttattgattgaaattgactaa

SEQ ID NO: 20 - protein, 1137 aa, Lactococcus lactis
MKKLLVANRGEIAVRVFRACNELGLSTVAVYAREDEYSVHRFKADESYLIGQGKKPIDAYLDIDDIIRVALESGADAIHPGYGLLSENLEFATKVRAAGLVFVGPELHHLDIFGDKIKAKAAADEAQVPGIPGTNGAVDIDGALEFAQTYGYPVMIKAALGGGGRGMRVARNDAEMHDGYARAKSEAIGAFGSGEIYVEKYIENPKHIEVQILGDSHGNIVHLHERDCSVQRRNQKVIEIAPAVGLSPEFRNEICEAAVKLCKNVGYVNAGTVEFLVKDDKFYFIEVNPRVQVEHTITELITGVDIVQAQILIAQGKDLHTEIGIPAQAEIPLLGSAIQCRITTEDPQNGFLPDTGKIDTYRSPGGFGIRLDVGNAYAGYEVTPYFDSLLVKVCTFANEFSDSVRKMDRVLHEFRIRGVKTNIPFLINVIANENFTSGQATTTFIDNTPSLFNFPRLRDRGTKTLHYLSMITVNGFPGIENTEKRHFEEPRQPLLNIEKKKTAKNILDEQGADAVVEYVKNTKEVLLTDTTLRDAHQSLLATRLRLQDMKGIAQAIDQGLPELFSAEMWGGATFDVAYRFLNESPWYRLRKLRKLMPNTMFQMLFRGSNAVGYQNYPDNVIEEFIHVAAHEGIDVFRIFDSLNWLPQMEKSIQAVRDNGKIAEATICYTGDILDPSRPKYNIQYYKDLAKELEATGAHILAVKDMAGLLKPQAAYRLISELKDTVDLPIHLHTHDTSGNGIITYSGATQAGVDIIDVATASLAGGTSQPSMQSIYYALEHGPRHASINVKNAEQIDHYWEDVRKYYAPFEAGITSPQTEVYMHEMPGGQYTNLKSQAAAVGLGHRFDEIKQMYRKVNMMFGDIIKVTPSSKVVGDMALFMIQNELTEEDVYARGNELNFPESVVSFFRGDLGQPVGGFPEELQKIIVKDKSVIMDRPGLHAEKVDFATVKADLEQKIGYEPGDHEVISYIMYPQVFLDYQKMQREFGAVTLLDTPTFLHGMRLNEKIEVQIEKGKTLSIRLDEIGEPDLAGNRVLFFNLNGQRREVVINDQSVQTQIVAKRKAETGNPNQIGATMPGSVLEILVKAGDKVKKGQALMVTEAMKMETTIESPFDGEVIALHVVKGEAIQTQDLLIEID

SEQ ID NO: 21 - DNA, 1080 bp, Sulfolobus tokodaii
atgatcctgatgcgccgcaccctcaaagcagcaatcctgggcgccacgggcttggttggtattgagtacgtgcgcatgctgagcaatcacccgtatatcaaaccagcatatctggcgggtaagggcagcgttggcaagccttacggtgaggtcgtgcgctggcagacggtaggtcaggtgccgaaagaaattgcggacatggagatcaagccgacggacccgaagctgatggatgacgttgacattatcttctccccgctgccgcagggtgcagctggtccggtggaagaacaatttgccaaagaaggttttcctgttattagcaacagcccggaccatcgctttgatccggacgttccgctgctggtgccggagctgaatccgcatacgatcagcttgattgacgagcaacgtaagcgtcgcgagtggaaaggttttatcgtcactacgccgctgtgcaccgcccaaggtgcggccattccgctgggcgcaatcttcaaagattacaagatggacggtgcgtttatcaccaccatccagagcctgagcggcgctggctatccgggtattccgtccctggatgtggttgataacattctgccgctgggcgatggttacgacgccaagaccattaaagaaatcttccgtatcctgagcgaggttaaacgtaatgttgacgagccgaaactggaggatgtgtctctggcggcgaccacgcaccgtatcgcgaccattcacggtcattacgaagtcctgtatgtgagcttcaaagaagaaactgcagcggagaaggtcaaagaaaccctggagaacttccgtggcgagcctcaggatttgaagttgccgaccgcgccatcgaaaccgattattgtcatgaacgaagatacccgtccgcaggtttacttcgaccgttgggcgggtgatatcccgggtatgagcgttgtcgtcggtcgtctgaagcaagtgaacaagcgtatgattcgtctggttagcctgattcacaataccgtgcgtggcgctgcgggtggtggcatcctggcagcggagctgttggtcgagaaaggctatattgaaaagtaa

SEQ ID NO: 22 - protein, 359 aa, Sulfolobus tokodaii
MILMRRTLKAAILGATGLVGIEYVRMLSNHPYIKPAYLAGKGSVGKPYGEVVRWQTVGQVPKEIADMEIKPTDPKLMDDVDIIFSPLPQGAAGPVEEQFAKEGFPVISNSPDHRFDPDVPLLVPELNPHTISLIDEQRKRREWKGFIVTTPLCTAQGAAIPLGAIFKDYKMDGAFITTIQSLSGAGYPGIPSLDVVDNILPLGDGYDAKTIKEIFRILSEVKRNVDEPKLEDVSLAATTHRIATIHGHYEVLYVSFKEETAAEKVKETLENFRGEPQDLKLPTAPSKPIIVMNEDTRPQVYFDRWAGDIPGMSVVVGRLKQVNKRMIRLVSLIHNTVRGAAGGGILAAELLVEKGYIEK

SEQ ID NO: 23 - DNA, 3792 bp, Pseudonacardia dioxanivorans
atgtccaccagcagtacctccggccagacgagccagttcggccccaacgaatggctcgtcgaggagatgtaccagcgtttcctcgacgacccggatgccgtcgacgccgcctggcacgacttcttcgccgactaccggccgccgtccggtgacgacgagacggagtcgaacggaaccacctccaccacgacgaccccgaccgcctccgcgtccgccgccgctccccgttccgccgccgcctccgggacggccgcggcgaacggctcggcgccggcccccgaggacaaggcggagaagaccaccgagaagaccgtgcagcagcccgccacgcagaagccggcccagcaggccgaccggtcggcgaacggcgccgcccccggcaagcccgtcgcgggcaccacgtcgcgtgccgccaagcccgcgcccgccgccgccgagggcgaggtgctgcccctgcgcggggcggcgaacgccgtcgtcaagaacatgaacgcctcgctcgccgtgccgaccgcgacgagcgtgcgcgccgtgccggcgaagctcatcgccgacaaccgcatcgtcatcaacaaccagctcaagcgcacgcgtggcggcaagctgtcgttcacccacctcatcggctacgcggtggtcaaggcgctggccgacttcccggtgatgaaccggcacttcgtcgaggtcgacgggaaacccaccgccgtccagccggagcacgtcaacctcggcctcgcgatcgacctgcagggcaagaacgggcagcgttccctcgtcgtcgtgtcgatcaagggctgcgaggagatgaccttcgcgcagttctggtccgcctacgagagcatggtccacaaggcgcgcaacggcacgctcgccgccgaggacttcgcgggcaccacgatcagcctcaccaacccgggcaccctcggcaccaaccactcggtgccgcggttgatgcagggccagggcacgatcgtcggtgtcggcgcgatggagtaccccgccgagttccagggcgccagcgaggagcggctcgccgagctcggcatcagcaagatcatcacgctgacgtcgacctacgaccaccggatcatccagggcgcggagtcgggcgacttcctgcgccgggtccaccacctgctgctgggcggcgacgggttcttcgacgacatcttccgctccctgcgcgtcccgtacgagccgatccgctgggtgcaggacttcgccgagggcgaggtcgacaagaccgcgcgcgtcctcgagctgatcgagtcctaccgcacgcgcggccacctgatggccgacaccgacccgctcaactaccgccagcgccgtcaccccgacctcgacgtgctcagccacgggctgacgctgtgggacctcgaccgcgagttcgcggtcggcggcttcgcgggccagctgcggatgaagctgcgcgacgtgctcggtgtgctgcgcgacgcgtactgccgcaccatcggcaccgagtacatgcacatcgccgacccggagcagcgggcctggctgcaggagcgcatcgaggtcccgcaccagaagccgccggtcgtcgagcagaagtacatcctgtcgaagctcaacgccgccgaggcgttcgagaccttcctgcagacgaagtacgtcgggcagaagcggttctccctggagggcggcgagaccgtcatcccgctgctcgacgccgtgctggacaaggctgccgagcacgagctcgccgaggtcgtcatcggcatgccgcaccgcggccggctcaacgtgctggccaacatcgtcggcaagccgatcagccagatcttccgcgagttcgagggcaacctcgacccgggccaggcccacggctccggcgacgtcaagtaccacctcggcgccgagggcaagtacttccgcatgttcggcgacggcgagacggtcgtgtcgctggcgtccaacccgagccacctcgaggccgtcgaccccgtgctcgaggggatcgtccgggccaagcaggacctgctcgaccagggcgacggcgccttcccggtgctgcccctgatgctgcacggcgacgccgcgttcgccgggcagggcgtcgtggccgagacgctgaacctcgccctgctgcgcggctaccgcaccggcggcaccgtgcacgtcgtcgtcaacaaccaggtcgggttcaccaccgcgcccgagcagtcgcgctcgtcgcagtactgcaccgacgtcgcgaagatgatcggcgcgccggtcttccacgtgaacggcgacgaccccgaggcgtgcgtgtgggtcgccaagctggcggtcgagtaccgcgagcgctggaacaacgacgtcgtgatcgacatgatctgctaccggcgccgcggccacaacgagggcgacgacccctcgatgacgcagccggcgatgtacgacgtcatcgacgccaagcgcagcgtccgcaagatctacaccgagtccctgatcggccgcggcgacatcaccgtcgacgaggccgaggccgcgctgaaggacttctccaaccagctcgagcacgtgttcaacgaggtccgcgagctggagcgcacgccgccgacgctctcgccctcggtcgagaacgagcagtcggtgcccaccgacctcgacacctcggtgccgctggaggtcatccaccgcatcggcgacacccacgtgcagctgccggaaggcttcaccgtgcaccagcgggtcaagccggtgctggccaagcgggagaagatgtcgcgcgagggcgacgtcgactgggccttcggcgagctgctcgccatgggctcgctggcgctcaacggcaagctggtccggctctccgggcaggactcgcggcgcggcacgttcgtgcagcggcactcggtcgtcatcgaccgcaagaccggcgaggagtacttcccgctgcgcaacctcgccgaggaccagggccgcttcctgccctacgactcggcgctgtcggagtacgcggcgctcggcttcgagtacggctactccgtggccaacccggacgcgctcgtcatgtgggaggcgcagttcggcgacttcgtcaacggcgcccagtcgatcatcgacgagttcatctcctccggtgaggccaagtgggggcagatggccgacgtcgtgctgctgctgccgcacggcctcgagggccagggccccgaccacagctccggacgcatcgagcggttcctgcagctgtgtgccgaggggtcgatgacggtcgcgatgccgtcggagcccgcgaaccacttccacctgctgcgccggcacgccctcgacggggtgcgccgcccgctggtggtattcacgccgaagtggatgctgcgcgccaagcaggtcgtcagcccgctgtcggacttcaccggtggccgcttccgcaccgtgatcgacgacccgcgcttccgcaactccgacagccccgcccccggggtgcgccgggtgctgctgtgctcgggcaagatctactgggagctggcggcggcgatggagaagcgcggcgggcgcgacgacatcgcgatcgtccgcatcgagcagctctacccggtgcccgaccgccagctcgccgcggtcctcgagcgctaccccaacgccgacgacatccgctgggtccaggaggagccggccaaccagggcgcgtggccgttcttcggcctcgacctgcgggagaagctcccggagcggctctcgggcctgacccgcgtgtcgcggcgccggatggccgcgcccgcggccggctcgtcgaaggtccacgaggtcgagcaggccgcgatcctcgacgaggcgctgagctga

SEQ ID NO: 24 - protein, 1263 aa, Pseudonacardia dioxanivorans
MSTSSTSGQTSQFGPNEWLVEEMYQRFLDDPDAVDAAWHDFFADYRPPSGDDETESNGTTSTTTTPTASASAAAPRSAAASGTAAANGSAPAPEDKAEKTTEKTVQQPATQKPAQQADRSANGAAPGKPVAGTTSRAAKPAPAAAEGEVLPLRGAANAVVKNMNASLAVPTATSVRAVPAKLIADNRIVINNQLKRTRGGKLSFTHLIGYAVVKALADFPVMNRHFVEVDGKPTAVQPEHVNLGLAIDLQGKNGQRSLVVVSIKGCEEMTFAQFWSAYESMVHKARNGTLAAEDFAGTTISLTNPGTLGTNHSVPRLMQGQGTIVGVGAMEYPAEFQGASEERLAELGISKIITLTSTYDHRIIQGAESGDFLRRVHHLLLGGDGFFDDIFRSLRVPYEPIRWVQDFAEGEVDKTARVLELIESYRTRGHLMADTDPLNYRQRRHPDLDVLSHGLTLWDLDREFAVGGFAGQLRMKLRDVLGVLRDAYCRTIGTEYMHIADPEQRAWLQERIEVPHQKPPVVEQKYILSKLNAAEAFETFLQTKYVGQKRFSLEGGETVIPLLDAVLDKAAEHELAEVVIGMPHRGRLNVLANIVGKPISQIFREFEGNLDPGQAHGSGDVKYHLGAEGKYFRMFGDGETVVSLASNPSHLEAVDPVLEGIVRAKQDLLDQGDGAFPVLPLMLHGDAAFAGQGVVAETLNLALLRGYRTGGTVHVVVNNQVGFTTAPEQSRSSQYCTDVAKMIGAPVFHVNGDDPEACVWVAKLAVEYRERWNNDVVIDMICYRRRGHNEGDDPSMTQPAMYDVIDAKRSVRKIYTESLIGRGDITVDEAEAALKDFSNQLEHVFNEVRELERTPPTLSPSVENEQSVPTDLDTSVPLEVIHRIGDTHVQLPEGFTVHQRVKPVLAKREKMSREGDVDWAFGELLAMGSLALNGKLVRLSGQDSRRGTFVQRHSVVIDRKTGEEYFPLRNLAEDQGRFLPYDSALSEYAALGFEYGYSVANPDALVMWEAQFGDFVNGAQSIIDEFISSGEAKWGQMADVVLLLPHGLEGQGPDHSSGRIERFLQLCAEGSMTVAMPSEPANHFHLLRRHALDGVRRPLVVFTPKWMLRAKQVVSPLSDFTGGRFRTVIDDPRFRNSDSPAPGVRRVLLCSGKIYWELAAAMEKRGGRDDIAIVRIEQLYPVPDRQLAAVLERYPNADDIRWVQEEPANQGAWPFFGLDLREKLPERLSGLTRVSRRRMAAPAAGSSKVHEVEQAAILDEALS

SEQ ID NO: 25 - DNA, 1152 bp, Escherichia coli
atgatggctaacagaatgctggtgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaagggctttccccgaccaataaacccagtgtaccgattctggcaattcctaccacagcaggtactgcggcagaagtgaccattaactacgtgatcactgacgaagagaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgttgggttagggttggtgcatggtatggcgcatccactgggcgcgttttataacactccacacggtgttgcgaacgccatcctgttaccgcatgtcatgcgttataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggcgcaggcggcactggatgatgtttgtaccggtggcaacccgcgtgaagcaacgcttgaggatattgtagagctttaccataccgcctggtaa

SEQ ID NO: 26 - protein, 383 aa, Escherichia coli
MMANRMLVNETAWFGRGAVGALTDEVKRRGYQKALIVTDKTLVQCGVVAKVTDKMDAAGLAWAIYDGVVPNPTITVVKEGLGVFQNSGADYLIAIGGGSPQDTCKAIGIISNNPEFADVRSLEGLSPTNKPSVPILAIPTTAGTAAEVTINYVITDEEKRRKFVCVDPHDIPQVAFIDADMMDGMPPALKAATGVDALTHAIEGYITRGAWALTDALHIKAIEIIAGALRGSVAGDKDAGEEMALGQYVAGMGFSNVGLGLVHGMAHPLGAFYNTPHGVANAILLPHVMRYNADFTGEKYRDIARVMGVKVEGMSLEEARNAAVEAVFALNRDVGIPPHLRDVGVRKEDIPALAQAALDDVCTGGNPREATLEDIVELYHTAW

SEQ ID NO: 27 - DNA, 1770 bp, Ralstonia sp.S-6
atggcgaccggcaagggcgcagcagcatcgacgcaggagggcaagagccaaccgtttaaggtgactccgggtccgtttgacccggcgacgtggctggaatggagccgccaatggcagggtaccgaaggcaatggccacgcagcggccagcggcattccgggtctggatgccctggctggcgtgaagattgcaccggcgcaattgggcgacattcaacagcgctatatgaaagacttcagcgccctgtggcaagcgatggcggagggcaaagcggaggcaaccggtccgctgcacgatcgtcgcttcgcgggtgacgcgtggcgtacgaacctgccgtaccgctttgcagccgcattttacctgttgaatgcccgtgccttgaccgaactggcggacgcggtcgaggcagatgcgaaaacccgtcaacgtattcgtttcgcgatcagccaatgggttgacgcaatgagcccagcaaacttcctggcgacgaacccggaggcgcagcgccgtctgatcgaaagcaacggcgagagcctgcgtgctggtctgcgcaacatgctggaggacctgacccgtggtaaaatctcccaaaccgatgaaagcgccttcgaagttggtcgcaacgtcgcggtcaccgagggtgctgtggtttacgaaaatgagtattttcagctgctgcagtacaagccgttgaccgcgaaagtgcacgcgcgtccgctgctgatggtgccgccgtgcatcaataagtattacatcctggatctgcagccggaatccagcctggtccgccatatcgttgagcagggccatacggttttcctggtgagctggcgtaacccggatgcgagcatggcagcgcgtacctgggatgactatatcgagcatggcgccattcgtgccattgaagtggcgcgtgctatcagcggtcagccgcgcattaatgtcctgggtttttgcgtgggcggtaccattgtctccactgcgctggcagttatggccggtcgtggcgaacgtccagcccagagcctgacgctgctgaccacgctgttggatttctccgatactggtgtgttggacgtttttgtcgacgaagcacatgttcagttgcgtgaggcgaccctgggcggtgctgcaggtgcgccgtgtgcgctgctgcgtggtatcgagttggcgaatacctttagcttcctgcgcccgaacgatctggtttggaattatgtggttgacaattacctgaagggcaacaccccggtgccatttgatctgttgttctggaacggtgacgcgaccaacctgccgggtccgtggtattgttggtatctgcgccatacgtacctgcaagacgagctgaaggttccgggtaagctgaccgtttgcggcgtacctgtggacctgggtaaaatcgacgtcccgacgtacctgtatggtagccgtgaggatcacatcgtcccgtggaccgcggcttacgcgtctacgcgtttgctgagcaacgatctgcgtttcgtcctgggtgcatctggtcacatcgccggtgtgattaatccaccagccaaaaacaaacgcagccactggacgaatgatgcgctgccggaaagcccgcagcagtggctggcaggtgcgattgagcaccacggctcttggtggccggactggaccgcatggctggccggtcaagctggtgcgaaacgtgcggctccggccaattacggcaatgcgcgttaccgcgctattgaaccggcacctggtcgttacgttaaagcaaaggcgtaa

SEQ ID NO: 28 - protein, 589 aa, Ralstonia sp.S-6
MATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRRLIESNGESLRAGLRNMLEDLTRGKISQTDESAFEVGRNVAVTEGAVVYENEYFQLLQYKPLTAKVHARPLLMVPPCINKYYILDLQPESSLVRHIVEQGHTVFLVSWRNPDASMAARTWDDYIEHGAIRAIEVARAISGQPRINVLGFCVGGTIVSTALAVMAGRGERPAQSLTLLTTLLDFSDTGVLDVFVDEAHVQLREATLGGAAGAPCALLRGIELANTFSFLRPNDLVWNYVVDNYLKGNTPVPFDLLFWNGDATNLPGPWYCWYLRHTYLQDELKVPGKLTVCGVPVDLGKIDVPTYLYGSREDHIVPWTAAYASTRLLSNDLRFVLGASGHIAGVINPPAKNKRSHWTNDALPESPQQWLAGAIEHHGSWWPDWTAWLAGQAGAKRAAPANYGNARYRAIEPAPGRYVKAKA

SEQ ID NO: 29 - DNA, 3420 bp, Typanosoma brucei
atggtagacggccgcagcagcgcatccatcgtcgcagtcgacccggagcgtgccgcacgcgaacgcgatgcggctgcgcgtgccctgttgcaggacagcccgctgcacacgaccatgcagtatgcgacctcgggtctggagctgactgtgccgtatgcactgaaagttgtggcaagcgctgatacctttgatcgtgcaaaggaagtggcggacgaagtcctgcgctgcgcatggcaattggcagataccgttctgaacagctttaaccctaacagcgaggtgagcctggtcggtcgcctgccggttggtcaaaaacatcagatgtccgcaccgctgaaacgtgtcatggcgtgttgccagcgcgtgtacaactccagcgccggttgcttcgacccgagcacggcgccagtcgcaaaagccttgcgcgaaattgcactgggtaaggagcgcaataacgcttgcctggaggcgctgacccaggcttgtaccctgccgaacagcttcgttatcgatttcgaagcgggcaccatcagccgcaaacacgaacatgcaagcctggacctgggtggcgtttcgaaaggctatatcgtggattatgtgattgacaacatcaatgccgctggtttccagaatgttttcttcgattggggtggtgactgtcgtgcctccggtatgaatgcgcgcaatacgccgtgggtcgtcggtattactcgcccaccgagcttggatatgctgccgaacccgccaaaggaagcgagctatatcagcgtcatctccctggacaacgaggcgttggcgaccagcggtgattacgagaacctgatctacaccgcagacgataagccgttgacctgcacttacgattggaaaggtaaagagctgatgaagccgagccagagcaatatcgctcaagttagcgtgaaatgctacagcgcaatgtacgccgatgccctggcaacggcgtgctttatcaagcgtgacccggcgaaagttcgtcaactgctggacggttggcgttatgttcgcgacacggtccgtgattaccgtgtgtacgtgcgtgagaatgagcgtgtagctaagatgttcgagattgcgactgaagatgcggagatgcgtaagcgtcgtattagcaatactctgcctgcacgtgtgatcgtggttggtggcggtctggcgggtctgagcgctgcgatcgaagctgcgggctgtggtgcgcaggtggtcctgatggagaaggaagccaagctgggcggtaacagcgcgaaagctaccagcggtatcaacggctggggcacccgtgcgcaggctaaagcgagcattgttgatggcggcaagtactttgaacgtgacacttacaaatcgggtattggcggtaatactgatccggcactggtcaaaaccctgtccatgaagagcgcggacgcgattggttggctgaccagcctgggcgtcccgctgaccgtcctgagccagctgggtggccatagccgcaagcgcacccatcgtgcaccggacaagaaagacggcacgcctctgccaatcggctttaccatcatgaaaactctggaggatcacgtccgtggtaatctgtctggccgtatcaccatcatggagaattgtagcgttaccagcctgctgagcgaaaccaaggaacgcccggacggcacgaagcagatccgtgtgacgggtgtcgagtttacccaagcgggctctggcaagaccaccatcttggcggatgcggttatcctggccacgggtggtttcagcaatgacaagacggctgatagcctgctgcgcgaacacgcaccgcacctggttaactttccgaccaccaacggcccgtgggcgacgggtgatggtgtgaagttggctcagcgtctgggtgctcaactggtcgatatggataaagttcagctgcacccgaccggcctgattaatccgaaagacccggccaatccgaccaaattcctgggtcctgaagcgttgcgtggtagcggtggtgtgctgctgaataaacaaggtaaacgttttgtgaatgagctggatctgcgtagcgtggttagcaaagccattatggagcaaggtgccgagtatccgggcagcggtggcagcatgttcgcgtattgtgttctgaacgctgcggcacaaaaactgttcggcgtttcttcgcatgagttttactggaaaaagatgggcttgttcgtgaaggccgataccatgcgcgacctggcggctctgatcggttgtccggttgagagcgtccaacaaacgctggaagagtatgaacgtctgagcattagccaacgcagctgcccgatcacccgtaagtctgtgtacccgtgtgttctgggtacgaaaggcccgtactatgtggcgttcgtgaccccgagcattcactatacgatgggcggttgtttgatcagcccgagcgcggagatccaaatgaagaacaccagctctcgtgcgccgctgtcccatagcaacccgatcctgggtctgtttggcgcaggcgaagtgaccggcggtgtgcacggtggtaaccgcctgggcggcaacagcttgctggagtgcgtcgtctttggtcgtattgcaggtgaccgtgcgagcaccattctgcaacgcaagtctagcgcactgtcctttaaagtttggaccaccgtcgttctgcgtgaggttcgcgagggtggtgtctatggtgcgggcagccgtgtgctgcgttttaacctgccaggcgcgctgcaacgctctggtctgtccctgggccagttcatcgcgattcgtggtgattgggacggtcaacagttgattggctattactccccgattaccctgcctgacgacctgggtatgattgacattctggcacgcagcgacaagggtacgctgcgtgagtggattagcgcgctggaaccgggtgacgcggtggagatgaaagcgtgtggtggcctggtgattgagcgtcgtctgagcgataagcacttcgtgtttatgggccacatcatcaataaactgtgcttgattgccggtggtacgggtgttgcaccgatgctgcaaatcatcaaagcggcattcatgaagccgtttatcgatacgttggaaagcgttcatctgatctatgcggccgaggatgttactgaattgacctaccgcgaagttttggaggagcgtcgccgtgaaagccgtggtaaattcaaaaagacgttcgtgttgaaccgtcctccgccgctgtggacggatggtgtcggctttattgaccgtggcattctgaccaatcatgttcagccgccgtccgacaatctgctggtggccatttgtggtccgcctgtgatgcaacgcattgttaaagcgaccctgaaaaccctgggttacaatatgaatctggttcgtaccgtggacgaaacggaaccgagcggtagctaa

SEQ ID NO: 30 - protein, 1139 aa, Typanosoma brucei
MVDGRSSASIVAVDPERAARERDAAARALLQDSPLHTTMQYATSGLELTVPYALKVVASADTFDRAKEVADEVLRCAWQLADTVLNSFNPNSEVSLVGRLPVGQKHQMSAPLKRVMACCQRVYNSSAGCFDPSTAPVAKALREIALGKERNNACLEALTQACTLPNSFVIDFEAGTISRKHEHASLDLGGVSKGYIVDYVIDNINAAGFQNVFFDWGGDCRASGMNARNTPWVVGITRPPSLDMLPNPPKEASYISVISLDNEALATSGDYENLIYTADDKPLTCTYDWKGKELMKPSQSNIAQVSVKCYSAMYADALATACFIKRDPAKVRQLLDGWRYVRDTVRDYRVYVRENERVAKMFEIATEDAEMRKRRISNTLPARVIVVGGGLAGLSAAIEAAGCGAQVVLMEKEAKLGGNSAKATSGINGWGTRAQAKASIVDGGKYFERDTYKSGIGGNTDPALVKTLSMKSADAIGWLTSLGVPLTVLSQLGGHSRKRTHRAPDKKDGTPLPIGFTIMKTLEDHVRGNLSGRITIMENCSVTSLLSETKERPDGTKQIRVTGVEFTQAGSGKTTILADAVILATGGFSNDKTADSLLREHAPHLVNFPTTNGPWATGDGVKLAQRLGAQLVDMDKVQLHPTGLINPKDPANPTKFLGPEALRGSGGVLLNKQGKRFVNELDLRSVVSKAIMEQGAEYPGSGGSMFAYCVLNAAAQKLFGVSSHEFYWKKMGLFVKADTMRDLAALIGCPVESVQQTLEEYERLSISQRSCPITRKSVYPCVLGTKGPYYVAFVTPSIHYTMGGCLISPSAEIQMKNTSSRAPLSHSNPILGLFGAGEVTGGVHGGNRLGGNSLLECVVFGRIAGDRASTILQRKSSALSFKVWTTVVLREVREGGVYGAGSRVLRFNLPGALQRSGLSLGQFIAIRGDWDGQQLIGYYSPITLPDDLGMIDILARSDKGTLREWISALEPGDAVEMKACGGLVIERRLSDKHFVFMGHIINKLCLIAGGTGVAPMLQIIKAAFMKPFIDTLESVHLIYAAEDVTELTYREVLEERRRESRGKFKKTFVLNRPPPLWTDGVGFIDRGILTNHVQPPSDNLLVAICGPPVMQRIVKATLKTLGYNMNLVRTVDETEPSGS

SEQ ID NO: 31 - DNA, 1362 bp, Clostridium kluyveri
atgtccaacgaggttagcattaaggagctgattgagaaggcgaaagtggcgcagaaaaagctggaagcgtatagccaagagcaagttgacgttctggtcaaggcgctgggtaaagttgtgtacgacaacgccgagatgttcgcgaaagaggcggtggaggaaaccgagatgggtgtttacgaggataaagtggctaaatgtcatctgaaatctggtgcaatctggaatcacattaaagataagaaaaccgttggtattatcaaggaagaaccggagcgtgcgctggtgtacgtcgcgaagcctaaaggtgttgtggcggcgacgacccctatcaccaatcctgtggttaccccgatgtgtaacgcgatggcagcaattaaaggtcgcaacaccatcattgtcgccccgcatccgaaggcgaagaaggtgagcgcgcacaccgtggagctgatgaatgcagaactgaaaaagttgggtgcgccggaaaacattatccagatcgttgaagccccaagccgtgaagcagccaaggagttgatggagagcgcagacgtggttatcgccacgggtggcgcaggccgtgttaaagcagcgtactcctccggccgtccggcatacggtgtcggtccgggcaattctcaggtcattgtcgataagggttacgattataacaaagctgcccaggacatcattaccggccgcaagtatgacaacggtatcatttgcagctctgagcagagcgtgatcgcaccggcggaggactacgacaaggtcatcgcggctttcgtcgagaatggcgcgttctatgtcgaggatgaggaaactgtggagaaattccgtagcacgctgttcaaggatggcaagatcaatagcaaaatcatcggtaaatccgtgcagatcatcgctgacctggctggtgtcaaggtgccggaaggcaccaaggtgatcgtgttgaagggcaagggtgccggtgaaaaggacgttctgtgcaaggagaaaatgtgcccggtcctggttgccctgaaatatgacacctttgaggaggcggtcgagatcgcgatggccaactatatgtacgagggtgcgggccataccgccggtatccacagcgataacgacgagaatatccgctacgcgggtacggtgctgccaatcagccgtctggttgtcaaccagccagcaactacggccggtggtagctttaacaatggttttaatccgaccaccaccttgggctgcggtagctggggccgtaactccattagcgagaacctgacgtatgagcatctgattaatgtcagccgtattggctatttcaataaggaggcaaaagttcctagctacgaggagatctggggttaa

SEQ ID NO: 32 - protein, 453 aa, Clostridium kluyveri
MSNEVSIKELIEKAKVAQKKLEAYSQEQVDVLVKALGKVVYDNAEMFAKEAVEETEMGVYEDKVAKCHLKSGAIWNHIKDKKTVGIIKEEPERALVYVAKPKGVVAATTPITNPVVTPMCNAMAAIKGRNTIIVAPHPKAKKVSAHTVELMNAELKKLGAPENIIQIVEAPSREAAKELMESADVVIATGGAGRVKAAYSSGRPAYGVGPGNSQVIVDKGYDYNKAAQDIITGRKYDNGIICSSEQSVIAPAEDYDKVIAAFVENGAFYVEDEETVEKFRSTLFKDGKINSKIIGKSVQIIADLAGVKVPEGTKVIVLKGKGAGEKDVLCKEKMCPVLVALKYDTFEEAVEIAMANYMYEGAGHTAGIHSDNDENIRYAGTVLPISRLVVNQPATTAGGSFNNGFNPTTTLGCGSWGRNSISENLTYEHLINVSRIGYFNKEAKVPSYEEIWG

SEQ ID NO: 33 - DNA, 870 bp, Arabidopsis thaliana
atggaagtaggttttctgggtctgggcattatgggtaaagctatgtccatgaacctgctgaaaaacggtttcaaagttaccgtgtggaaccgcactctgtctaaatgtgatgaactggttgaacacggtgcaagcgtgtgcgagtctccggctgaggtgatcaagaaatgcaaatacacgatcgcgatgctgagcgatccgtgtgcagctctgtctgttgttttcgataaaggcggtgttctggaacagatctgcgagggtaagggctacatcgacatgtctaccgtcgacgcggaaactagcctgaaaattaacgaagcgatcacgggcaaaggtggccgttttgtagaaggtcctgttagcggttccaaaaagccggcagaagacggccagctgatcatcctggcagcaggcgacaaagcactgttcgaggaatccatcccggcctttgatgtactgggcaaacgttccttttatctgggtcaggtgggtaacggtgcgaaaatgaaactgattgttaacatgatcatgggttctatgatgaacgcgtttagcgaaggtctggtactggcagataaaagcggtctgtctagcgacacgctgctggatattctggatctgggtgctatgacgaatccgatgttcaaaggcaaaggtccgtccatgactaaatccagctacccaccggctttcccgctgaaacaccagcagaaagacatgcgtctggctctggctctgggcgacgaaaacgctgttagcatgccggtcgctgcggctgcgaacgaagccttcaagaaagcccgtagcctgggcctgggcgatctggacttttctgctgttatcgaagcggtaaaattctctcgtgaataa

SEQ ID NO: 34 - protein, 289 aa, Arabidopsis thaliana
MEVGFLGLGIMGKAMSMNLLKNGFKVTVWNRTLSKCDELVEHGASVCESPAEVIKKCKYTIAMLSDPCAALSVVFDKGGVLEQICEGKGYIDMSTVDAETSLKINEAITGKGGRFVEGPVSGSKKPAEDGQLIILAAGDKALFEESIPAFDVLGKRSFYLGQVGNGAKMKLIVNMIMGSMMNAFSEGLVLADKSGLSSDTLLDILDLGAMTNPMFKGKGPSMTKSSYPPAFPLKHQQKDMRLALALGDENAVSMPVAAAANEAFKKARSLGLGDLDFSAVIEAVKFSRE

SEQ ID NO: 35 - DNA, 1821 bp, Artificial Sequence
  Pseudomonas putida/Ralstonia eutropha fusion protein
atgactagaaggaggtttcatatgagtaacaagaacaacgatgagctggcgacgggtaaaggtgctgctgcatcttctactgaaggtaaatctcagccgtttaaattcccaccgggtccgctggacccggccacttggctggaatggagccgtcagtggcaaggtccggagggcaatggcggtaccgtgccgggtggctttccgggtttcgaagcgttcgcggcgtccccgctggcgggcgtgaaaatcgacccggctcagctggcagagatccagcagcgttatatgcgtgatttcaccgagctgtggcgtggtctggcaggcggtgacaccgagagcgctggcaaactgcatgaccgtcgcttcgcgtccgaagcgtggcacaaaaacgcgccgtatcgctatactgcggcattttacctgctgaacgcacgtgcactgacggaactggctgatgcagtagaagcggatccgaaaacccgtcagcgtatccgttttgcggtttcccagtgggtagatgctatgagcccggctaacttcctggccaccaacccggacgctcagaaccgtctgatcgagagccgtggtgaaagcctgcgtgccggcatgcgcaatatgctggaagatctgacccgcggtaaaatttcccaaaccgatgagactgccttcgaagtaggccgtaacatggcagttaccgaaggtgctgtggtattcgaaaacgagttcttccagctgctgcagtacaaacctctgactgacaaagtatacacccgtccgctgctgctggtaccgccgtgcattaacaagttctatattctggacctgcagccggaaggttctctggtccgttacgcagtcgaacagggtcacactgtattcctggtgagctggcgcaatccagacgctagcatggctggctgtacctgggatgactatattgaaaacgcggctatccgcgccatcgaggttgtgcgtgatatcagcggtcaggacaagatcaacaccctgggcttttgtgttggtggcacgatcatctccactgccctggcggtcctggccgcccgtggtgagcacccggtggcctctctgaccctgctgactaccctgctggacttcaccgatactggtatcctggatgttttcgtggacgagccacacgttcagctgcgtgaggcgactctgggcggcgccagcggcggtctgctgcgtggtgtcgagctggccaataccttttccttcctgcgcccgaacgacctggtttggaactacgttgttgacaactatctgaaaggcaacaccccggtacctttcgatctgctgttctggaacggtgatgcaaccaacctgcctggtccatggtactgttggtacctgcgtcatacttacctgcagaacgaactgaaagagccgggcaaactgaccgtgtgtaacgaacctgtggacctgggcgcgattaacgttcctacttacatctacggttcccgtgaagatcacatcgtaccgtggaccgcggcttacgccagcaccgcgctgctgaagaacgatctgcgtttcgtactgggcgcatccggccatatcgcaggtgtgatcaaccctcctgcaaagaaaaagcgttctcattggaccaacgacgcgctgccagaatccgcgcaggattggctggcaggtgctgaggaacaccatggttcctggtggccggattggatgacctggctgggtaaacaagccggtgcaaaacgtgcagctccaactgaatatggtagcaagcgttatgctgcaatcgagccagcgccaggccgttacgttaaagcgaaagcataa

SEQ ID NO: 36 - protein, 599 aa, Artificial Sequence
  Pseudomonas putida/Ralstonia eutropha fusion protein
MSNKNNDELATGKGAAASSTEGKSQPFKFPPGPLDPATWLEWSRQWQGPEGNGGTVPGGFPGFEAFAASPLAGVKIDPAQLAEIQQRYMRDFTELWRGLAGGDTESAGKLHDRRFASEAWHKNAPYRYTAAFYLLNARALTELADAVEADPKTRQRIRFAVSQWVDAMSPANFLATNPDAQNRLIESRGESLRAGMRNMLEDLTRGKISQTDETAFEVGRNMAVTEGAVVFENEFFQLLQYKPLTDKVYTRPLLLVPPCINKFYILDLQPEGSLVRYAVEQGHTVFLVSWRNPDASMAGCTWDDYIENAAIRAIEVVRDISGQDKINTLGFCVGGTIISTALAVLAARGEHPVASLTLLTTLLDFTDTGILDVFVDEPHVQLREATLGGASGGLLRGVELANTFSFLRPNDLVWNYVVDNYLKGNTPVPFDLLFWNGDATNLPGPWYCWYLRHTYLQNELKEPGKLTVCNEPVDLGAINVPTYIYGSREDHIVPWTAAYASTALLKNDLRFVLGASGHIAGVINPPAKKKRSHWTNDALPESAQDWLAGAEEHHGSWWPDWMTWLGKQAGAKRAAPTEYGSKRYAAIEPAPGRYVKAKA